SEQUENCE LISTING (346 sequences)

SEQ ID NO: 1 - protein, 10 aa, Artificial Sequence
  VH CDR1
FTISNNYWIC

SEQ ID NO: 2 - protein, 14 aa, Artificial Sequence
  VH CDR2
WIGCIATGDGSTYY

SEQ ID NO: 3 - protein, 14 aa, Artificial Sequence
  VH CDR3
RGAAGSSWTTYFDF

SEQ ID NO: 4 - protein, 8 aa, Artificial Sequence
  VL CDR1
QNIDNYLS

SEQ ID NO: 5 - protein, 11 aa, Artificial Sequence
  VL CDR2
LLIYRASTLAS

SEQ ID NO: 6 - protein, 13 aa, Artificial Sequence
  VL CDR3
QNNNGGSTFTGFP

SEQ ID NO: 7 - protein, 9 aa, Artificial Sequence
  VH CDR1
FDFSSNGMC

SEQ ID NO: 8 - protein, 15 aa, Artificial Sequence
  VH CDR2
WIACIYVDSSDNTNY

SEQ ID NO: 9 - protein, 12 aa, Artificial Sequence
  VH CDR3
RGYGYVGSAMDL

SEQ ID NO: 10 - protein, 8 aa, Artificial Sequence
  VL CDR1
QSINNELS

SEQ ID NO: 11 - protein, 11 aa, Artificial Sequence
  VL CDR2
LLIYGASTLES

SEQ ID NO: 12 - protein, 14 aa, Artificial Sequence
  VL CDR3
QSAYYSSSSSYANT

SEQ ID NO: 13 - protein, 9 aa, Artificial Sequence
  VH CDR1
FTISSIWIC

SEQ ID NO: 14 - protein, 15 aa, Artificial Sequence
  VH CDR2
WIACIYAGSDGGSYY

SEQ ID NO: 15 - protein, 22 aa, Artificial Sequence
  VH CDR3
RASNSWQYGYAGYGNYKDYFNL

SEQ ID NO: 16 - protein, 8 aa, Artificial Sequence
  VL CDR1
ESISNLLA

SEQ ID NO: 17 - protein, 11 aa, Artificial Sequence
  VL CDR2
VLIYKASALPS

SEQ ID NO: 18 - protein, 12 aa, Artificial Sequence
  VL CDR3
QSYYGSSDTGNT

SEQ ID NO: 19 - protein, 10 aa, Artificial Sequence
  VH CDR1
FSFSSSYWIS

SEQ ID NO: 20 - protein, 15 aa, Artificial Sequence
  VH CD2
WIACIAIGSSGTTYY

SEQ ID NO: 21 - protein, 11 aa, Artificial Sequence
  VH CD3
RSGDGYTYVEL

SEQ ID NO: 22 - protein, 8 aa, Artificial Sequence
  VL CDR1
QSISSYLA

SEQ ID NO: 23 - protein, 11 aa, Artificial Sequence
  VL CDR2
LLIYRASTLAS

SEQ ID NO: 24 - protein, 12 aa, Artificial Sequence
  VL CDR3
QNYYDIDDSDNT

SEQ ID NO: 25 - protein, 7 aa, Artificial Sequence
  VH CDR1
FSFSWIC

SEQ ID NO: 26 - protein, 15 aa, Artificial Sequence
  VH CDR2
WIACIYAGSSAKTYY

SEQ ID NO: 27 - protein, 22 aa, Artificial Sequence
  VH CDR3
RASNYYRYGVAGYADYTGYFNL

SEQ ID NO: 28 - protein, 8 aa, Artificial Sequence
  VL CDR1
QSISSWLA

SEQ ID NO: 29 - protein, 11 aa, Artificial Sequence
  VL CDR2
LLIYGTSTLAS

SEQ ID NO: 30 - protein, 11 aa, Artificial Sequence
  VL CDR3
QNYGGSSSGDA

SEQ ID NO: 31 - protein, 10 aa, Artificial Sequence
  VH CDR1
FSFSSNYWIC

SEQ ID NO: 32 - protein, 15 aa, Artificial Sequence
  VH CDR2
WIACIATGSSGSTYY

SEQ ID NO: 33 - protein, 21 aa, Artificial Sequence
  VH CDR3
RGEYTYGYVEYAIVTQYYFDL

SEQ ID NO: 34 - protein, 8 aa, Artificial Sequence
  VL CDR1
QNIYSNLA

SEQ ID NO: 35 - protein, 11 aa, Artificial Sequence
  VL CDR2
LLIYGASNLES

SEQ ID NO: 36 - protein, 12 aa, Artificial Sequence
  VL CDR3
QSADYIGSAYNA

SEQ ID NO: 37 - protein, 10 aa, Artificial Sequence
  VH CDR1
FSFSSSYYMC

SEQ ID NO: 38 - protein, 14 aa, Artificial Sequence
  VH CDR2
WIACIYTTYGGTWY

SEQ ID NO: 39 - protein, 12 aa, Artificial Sequence
  VH CDR3
RSSISDVTYFNL

SEQ ID NO: 40 - protein, 8 aa, Artificial Sequence
  VL CDR1
QSISNYLA

SEQ ID NO: 41 - protein, 11 aa, Artificial Sequence
  VL CDR2
LLIYRASTLES

SEQ ID NO: 42 - protein, 10 aa, Artificial Sequence
  VL CDR3
QSYYDNNNYA

SEQ ID NO: 43 - protein, 9 aa, Artificial Sequence
  VH CDR1
FTLSTYWVC

SEQ ID NO: 44 - protein, 15 aa, Artificial Sequence
  VH CDR2
WIGCIDTVSSGDTYF

SEQ ID NO: 45 - protein, 9 aa, Artificial Sequence
  VH CDR3
RRTGSGWTL

SEQ ID NO: 46 - protein, 10 aa, Artificial Sequence
  VL CDR1
QSVYNNNYLA

SEQ ID NO: 47 - protein, 11 aa, Artificial Sequence
  VL CDR2
LLIYWASKLAS

SEQ ID NO: 48 - protein, 12 aa, Artificial Sequence
  VL CDR3
LGAYVSNGWYFA

SEQ ID NO: 49 - protein, 9 aa, Artificial Sequence
  VH CDR1
FSFSSYWTC

SEQ ID NO: 50 - protein, 15 aa, Artificial Sequence
  VH CDR2
WLGCTDGGSSGDTYY

SEQ ID NO: 51 - protein, 8 aa, Artificial Sequence
  VH CDR3
RNLITWDL

SEQ ID NO: 52 - protein, 10 aa, Artificial Sequence
  VL CDR1
ESVYNNNQLS

SEQ ID NO: 53 - protein, 11 aa, Artificial Sequence
  VL CDR2
LLIYWASKLAS

SEQ ID NO: 54 - protein, 12 aa, Artificial Sequence
  VL CDR3
AGYKSSITDGNA

SEQ ID NO: 55 - protein, 9 aa, Artificial Sequence
  VH CDR1
FDFSTNIMC

SEQ ID NO: 56 - protein, 14 aa, Artificial Sequence
  VH CDR2
WIACIYAGDGSTYY

SEQ ID NO: 57 - protein, 22 aa, Artificial Sequence
  VH CDR3
RASTYWNYGYAGYGYYPGYFNL

SEQ ID NO: 58 - protein, 8 aa, Artificial Sequence
  VL CDR1
QSIISALA

SEQ ID NO: 59 - protein, 11 aa, Artificial Sequence
  VL CDR2
LLIYAASTLAS

SEQ ID NO: 60 - protein, 14 aa, Artificial Sequence
  VL CDR3
QTYAYSTKSNYGSV

SEQ ID NO: 61 - protein, 10 aa, Artificial Sequence
  VH CDR1
FSFSSGYDMC

SEQ ID NO: 62 - protein, 14 aa, Artificial Sequence
  VH CDR2
WIACIYTGDGSTYY

SEQ ID NO: 63 - protein, 13 aa, Artificial Sequence
  VH CDR3
REDVSSGDYTFNL

SEQ ID NO: 64 - protein, 8 aa, Artificial Sequence
  VL CDR1
EDIYSGLA

SEQ ID NO: 65 - protein, 11 aa, Artificial Sequence
  VL CDR2
LLIYKASNLAS

SEQ ID NO: 66 - protein, 12 aa, Artificial Sequence
  VL CDR3
QQGVTYSNVDNT

SEQ ID NO: 67 - protein, 10 aa, Artificial Sequence
  VH CDR1
FSFSSTYYMC

SEQ ID NO: 68 - protein, 15 aa, Artificial Sequence
  VH CDR2
WIACIYTGSTGSTYY

SEQ ID NO: 69 - protein, 21 aa, Artificial Sequence
  VH CDR3
RGDYTYAYAGGAHVTNYYFDL

SEQ ID NO: 70 - protein, 8 aa, Artificial Sequence
  VL CDR1
QNIYSNLA

SEQ ID NO: 71 - protein, 11 aa, Artificial Sequence
  VL CDR2
LLIFGASNLES

SEQ ID NO: 72 - protein, 13 aa, Artificial Sequence
  VL CDR3
QTADYSSSTDWGA

SEQ ID NO: 73 - protein, 9 aa, Artificial Sequence
  VH CDR1
FDFSSNGMC

SEQ ID NO: 74 - protein, 15 aa, Artificial Sequence
  VH CDR2
WIACIYVDSSDSTYY

SEQ ID NO: 75 - protein, 12 aa, Artificial Sequence
  VH CDR3
RGYGYVGSAMDL

SEQ ID NO: 76 - protein, 8 aa, Artificial Sequence
  VL CDR1
QSISNELS

SEQ ID NO: 77 - protein, 11 aa, Artificial Sequence
  VL CDR2
LLIYGASTLES

SEQ ID NO: 78 - protein, 14 aa, Artificial Sequence
  VL CDR3
QSAYYSSSSSYANT

SEQ ID NO: 79 - protein, 9 aa, Artificial Sequence
  VH CDR1
FTLSTYWVC

SEQ ID NO: 80 - protein, 15 aa, Artificial Sequence
  VH CDR2
WIGCIDTVSSGDTYF

SEQ ID NO: 81 - protein, 9 aa, Artificial Sequence
  VH CDR3
RRTGSGWTL

SEQ ID NO: 82 - protein, 10 aa, Artificial Sequence
  VL CDR1
QSVYNNNYLA

SEQ ID NO: 83 - protein, 11 aa, Artificial Sequence
  VL CDR2
LLIYWASKLAS

SEQ ID NO: 84 - protein, 12 aa, Artificial Sequence
  VL CDR3
LGAYVSNGWYFA

SEQ ID NO: 85 - protein, 10 aa, Artificial Sequence
  VH CDR1
FSFSSSYYMC

SEQ ID NO: 86 - protein, 14 aa, Artificial Sequence
  VH CDR2
WIACIYPDDGNTYY

SEQ ID NO: 87 - protein, 22 aa, Artificial Sequence
  VH CDR3
RALAYYAYVDGGHSYAINDFDL

SEQ ID NO: 88 - protein, 10 aa, Artificial Sequence
  VL CDR1
QSVFSNDYFS

SEQ ID NO: 89 - protein, 11 aa, Artificial Sequence
  VL CDR2
LLIYDASRLAS

SEQ ID NO: 90 - protein, 12 aa, Artificial Sequence
  VL CDR3
QGTYYSSAWYNA

SEQ ID NO: 91 - protein, 9 aa, Artificial Sequence
  VH CDR1
FDFSSNGMC

SEQ ID NO: 92 - protein, 15 aa, Artificial Sequence
  VH CDR2
WIACIYVDSSDNTNY

SEQ ID NO: 93 - protein, 12 aa, Artificial Sequence
  VH CDR3
RGYGYVGSAMDL

SEQ ID NO: 94 - protein, 8 aa, Artificial Sequence
  VL CDR1
QSISNELS

SEQ ID NO: 95 - protein, 11 aa, Artificial Sequence
  VL CDR2
LLIYGASTLES

SEQ ID NO: 96 - protein, 14 aa, Artificial Sequence
  VL CDR3
QSAYYSSSSSYANT

SEQ ID NO: 97 - protein, 10 aa, Artificial Sequence
  VH CDR1
IDFSSDYYMC

SEQ ID NO: 98 - protein, 15 aa, Artificial Sequence
  VH CDR2
WIACIYTGSSDDTYY

SEQ ID NO: 99 - protein, 9 aa, Artificial Sequence
  VH CDR3
RGGYGGKDL

SEQ ID NO: 100 - protein, 8 aa, Artificial Sequence
  VL CDR1
QSIGSLLA

SEQ ID NO: 101 - protein, 11 aa, Artificial Sequence
  VL CDR2
LLIYWASTLAS

SEQ ID NO: 102 - protein, 15 aa, Artificial Sequence
  VL CDR3
QCTYGSSGSSSYLNA

SEQ ID NO: 103 - protein, 9 aa, Artificial Sequence
  VH CDR1
FDFSSNGMC

SEQ ID NO: 104 - protein, 15 aa, Artificial Sequence
  VH CDR2
WIACIYVDSSDSTYY

SEQ ID NO: 105 - protein, 12 aa, Artificial Sequence
  VH CDR3
RGYGYVGSAMDL

SEQ ID NO: 106 - protein, 8 aa, Artificial Sequence
  VL CDR1
QSISNELA

SEQ ID NO: 107 - protein, 11 aa, Artificial Sequence
  VL CDR2
LLIYGASTLES

SEQ ID NO: 108 - protein, 14 aa, Artificial Sequence
  VL CDR3
QSAYYSSSSSYANT

SEQ ID NO: 109 - protein, 9 aa, Artificial Sequence
  VH CDR1
FTLSTYWVC

SEQ ID NO: 110 - protein, 15 aa, Artificial Sequence
  VH CDR2
WIGCIDTVSSGDTYF

SEQ ID NO: 111 - protein, 9 aa, Artificial Sequence
  VH CDR3
RRTGSRWTL

SEQ ID NO: 112 - protein, 10 aa, Artificial Sequence
  VL CDR1
ESVYNNNYLS

SEQ ID NO: 113 - protein, 11 aa, Artificial Sequence
  VL CDR2
LLIYQASTLAS

SEQ ID NO: 114 - protein, 12 aa, Artificial Sequence
  VL CDR3
LGAFVSNGWYFA

SEQ ID NO: 115 - protein, 10 aa, Artificial Sequence
  VH CDR1
FSFSSGYNIC

SEQ ID NO: 116 - protein, 15 aa, Artificial Sequence
  VH CDR2
LIACIYTSSSGSTYY

SEQ ID NO: 117 - protein, 22 aa, Artificial Sequence
  VH CDR3
RGEAYYAYGYVGYAYYHGAFDP

SEQ ID NO: 118 - protein, 8 aa, Artificial Sequence
  VL CDR1
HSISKYFS

SEQ ID NO: 119 - protein, 11 aa, Artificial Sequence
  VL CDR2
LLIYEASTLAS

SEQ ID NO: 120 - protein, 12 aa, Artificial Sequence
  VL CDR3
QSYYYGTSSSYA

SEQ ID NO: 121 - protein, 10 aa, Artificial Sequence
  VH CDR1
FSFSSSYYMC

SEQ ID NO: 122 - protein, 15 aa, Artificial Sequence
  VH CDR2
WIACIYAGSSGGTYY

SEQ ID NO: 123 - protein, 18 aa, Artificial Sequence
  VH CDR3
RAFSYYYSDGYTGYAYGL

SEQ ID NO: 124 - protein, 8 aa, Artificial Sequence
  VL CDR1
QSISSYLA

SEQ ID NO: 125 - protein, 11 aa, Artificial Sequence
  VL CDR2
LLIYRASTLAS

SEQ ID NO: 126 - protein, 12 aa, Artificial Sequence
  VL CDR3
QGAYYSSSSSYG

SEQ ID NO: 127 - protein, 8 aa, Artificial Sequence
  VH CDR1
FSFSGYDM

SEQ ID NO: 128 - protein, 13 aa, Artificial Sequence
  VH CDR2
WIACIHSSSGTYY

SEQ ID NO: 129 - protein, 17 aa, Artificial Sequence
  VH CDR3
RDFSYTDDYISYVYATD

SEQ ID NO: 130 - protein, 6 aa, Artificial Sequence
  VL CDR1
QGSSLA

SEQ ID NO: 131 - protein, 10 aa, Artificial Sequence
  VL CDR2
LLIYAASYLA

SEQ ID NO: 132 - protein, 13 aa, Artificial Sequence
  VL CDR3
QSTYYSSSTDIRA

SEQ ID NO: 133 - protein, 9 aa, Artificial Sequence
  VH CDR1
FSFSSYWIC

SEQ ID NO: 134 - protein, 14 aa, Artificial Sequence
  VH CDR2
WIACIYAGSSGTYY

SEQ ID NO: 135 - protein, 20 aa, Artificial Sequence
  VH CDR3
RAEYIDGYADYTYTTLYFDL

SEQ ID NO: 136 - protein, 7 aa, Artificial Sequence
  VL CDR1
QIYNNLA

SEQ ID NO: 137 - protein, 10 aa, Artificial Sequence
  VL CDR2
LLIYGASNLE

SEQ ID NO: 138 - protein, 11 aa, Artificial Sequence
  VL CDR3
QSADLTSSINV

SEQ ID NO: 139 - protein, 10 aa, Artificial Sequence
  VH CDR1
FSFNSNYWMC

SEQ ID NO: 140 - protein, 14 aa, Artificial Sequence
  VH CDR2
WIGCILFGNTDTYY

SEQ ID NO: 141 - protein, 12 aa, Artificial Sequence
  VH CDR3
RSVSGVGSAWNL

SEQ ID NO: 142 - protein, 10 aa, Artificial Sequence
  VL CDR1
ESVYNNNHLA

SEQ ID NO: 143 - protein, 11 aa, Artificial Sequence
  VL CDR2
LLIYLASILDS

SEQ ID NO: 144 - protein, 12 aa, Artificial Sequence
  VL CDR3
AGYKGITIDGSA

SEQ ID NO: 145 - protein, 10 aa, Artificial Sequence
  VH CDR1
FDFSSYYWIC

SEQ ID NO: 146 - protein, 15 aa, Artificial Sequence
  VH CDR2
WIACIYGGSSGSTYY

SEQ ID NO: 147 - protein, 18 aa, Artificial Sequence
  VH CDR3
RSLYTWRYADYAASTLNL

SEQ ID NO: 148 - protein, 10 aa, Artificial Sequence
  VL CDR1
QSVYNVNLLA

SEQ ID NO: 149 - protein, 11 aa, Artificial Sequence
  VL CDR2
LLIYETSKLES

SEQ ID NO: 150 - protein, 11 aa, Artificial Sequence
  VL CDR3
AGGYSSSKDNS

SEQ ID NO: 151 - protein, 10 aa, Artificial Sequence
  VH CDR1
FSFSSSYFMC

SEQ ID NO: 152 - protein, 14 aa, Artificial Sequence
  VH CDR2
WIACIYTGDGNNYY

SEQ ID NO: 153 - protein, 22 aa, Artificial Sequence
  VH CDR3
RGGSYYAYGYAGYDYYPDAFDY

SEQ ID NO: 154 - protein, 8 aa, Artificial Sequence
  VL CDR1
QSISSYLS

SEQ ID NO: 155 - protein, 11 aa, Artificial Sequence
  VL CDR2
LLIYRASTLAS

SEQ ID NO: 156 - protein, 12 aa, Artificial Sequence
  VL CDR3
QSYYYSSSGSYG

SEQ ID NO: 157 - protein, 9 aa, Artificial Sequence
  VH CDR1
FSFNSYYMC

SEQ ID NO: 158 - protein, 15 aa, Artificial Sequence
  VH CDR2
WIACISGGSSDNTYY

SEQ ID NO: 159 - protein, 13 aa, Artificial Sequence
  VH CDR3
RDIPRSGYFGCDL

SEQ ID NO: 160 - protein, 8 aa, Artificial Sequence
  VL CDR1
QNIYSNLA

SEQ ID NO: 161 - protein, 11 aa, Artificial Sequence
  VL CDR2
LLIYGASNLES

SEQ ID NO: 162 - protein, 12 aa, Artificial Sequence
  VL CDR3
QSTVYNSNYANT

SEQ ID NO: 163 - protein, 10 aa, Artificial Sequence
  VH CDR1
FSFSSSYWIY

SEQ ID NO: 164 - protein, 15 aa, Artificial Sequence
  VH CDR2
WIACIYTASRGSIYY

SEQ ID NO: 165 - protein, 19 aa, Artificial Sequence
  VH CDR3
RGPDYTYGYIGDALTRLDL

SEQ ID NO: 166 - protein, 10 aa, Artificial Sequence
  VL CDR1
QSVYDNNWLA

SEQ ID NO: 167 - protein, 11 aa, Artificial Sequence
  VL CDR2
LLIYAASTLSS

SEQ ID NO: 168 - protein, 14 aa, Artificial Sequence
  VL CDR3
AGGYSSTSDIEDNT

SEQ ID NO: 169 - protein, 10 aa, Artificial Sequence
  VH CDR1
FSFSSSYWIC

SEQ ID NO: 170 - protein, 15 aa, Artificial Sequence
  VH CDR2
WIACIYAGSSGDTYY

SEQ ID NO: 171 - protein, 21 aa, Artificial Sequence
  VH CDR3
RAEYIDGYADYTYTTLYYFDL

SEQ ID NO: 172 - protein, 8 aa, Artificial Sequence
  VL CDR1
ESINSWLA

SEQ ID NO: 173 - protein, 11 aa, Artificial Sequence
  VL CDR2
LLIYSASTLAS

SEQ ID NO: 174 - protein, 10 aa, Artificial Sequence
  VL CDR3
QSYYSFSRFA

SEQ ID NO: 175 - protein, 10 aa, Artificial Sequence
  VH CDR1
FSFSSGYWIC

SEQ ID NO: 176 - protein, 14 aa, Artificial Sequence
  VH CDR2
WIACIYTGVGATYY

SEQ ID NO: 177 - protein, 13 aa, Artificial Sequence
  VH CDR3
RDFGGSSGFYFNL

SEQ ID NO: 178 - protein, 8 aa, Artificial Sequence
  VL CDR1
QSISNALA

SEQ ID NO: 179 - protein, 11 aa, Artificial Sequence
  VL CDR2
LLIYSASTLES

SEQ ID NO: 180 - protein, 14 aa, Artificial Sequence
  VL CDR3
QNYYGSTSSSYGVA

SEQ ID NO: 181 - protein, 10 aa, Artificial Sequence
  VH CDR1
FSFSSSYYMC

SEQ ID NO: 182 - protein, 15 aa, Artificial Sequence
  VH CDR2
WIACIYAGSTFSTYY

SEQ ID NO: 183 - protein, 20 aa, Artificial Sequence
  VH CDR3
RSDSYYTYGYAGYAYAIFNL

SEQ ID NO: 184 - protein, 8 aa, Artificial Sequence
  VL CDR1
ESIYSNLA

SEQ ID NO: 185 - protein, 11 aa, Artificial Sequence
  VL CDR2
LLIYLASTLAS

SEQ ID NO: 186 - protein, 12 aa, Artificial Sequence
  VL CDR3
QSAYYSSSADIA

SEQ ID NO: 187 - protein, 10 aa, Artificial Sequence
  VH CDR1
LDFSSSYWIC

SEQ ID NO: 188 - protein, 14 aa, Artificial Sequence
  VH CDR2
WIGCIKTATETTVY

SEQ ID NO: 189 - protein, 12 aa, Artificial Sequence
  VH CDR3
KTYADNGGYINL

SEQ ID NO: 190 - protein, 8 aa, Artificial Sequence
  VL CDR1
QNIYNNLA

SEQ ID NO: 191 - protein, 11 aa, Artificial Sequence
  VL CDR2
LLIYGASNLES

SEQ ID NO: 192 - protein, 11 aa, Artificial Sequence
  VL CDR3
QSADLTSSINV

SEQ ID NO: 193 - protein, 10 aa, Artificial Sequence
  VH CDR1
FSFSSSYWIC

SEQ ID NO: 194 - protein, 15 aa, Artificial Sequence
  VH CDR2
WIACIYTASRDSIYY

SEQ ID NO: 195 - protein, 19 aa, Artificial Sequence
  VH CDR3
RGPYYSYAYIGDALTRLDL

SEQ ID NO: 196 - protein, 10 aa, Artificial Sequence
  VL CDR1
QSVYDNNWLA

SEQ ID NO: 197 - protein, 11 aa, Artificial Sequence
  VL CDR2
LLIYEASKLAS

SEQ ID NO: 198 - protein, 14 aa, Artificial Sequence
  VL CDR3
AGGYSSSSDIEDNT

SEQ ID NO: 199 - protein, 10 aa, Artificial Sequence
  VH CDR1
FSFNSNYYMC

SEQ ID NO: 200 - protein, 15 aa, Artificial Sequence
  VH CDR2
WIACIYTGIVVPTYY

SEQ ID NO: 201 - protein, 13 aa, Artificial Sequence
  VH CDR3
RDPYVGSSYIYNL

SEQ ID NO: 202 - protein, 10 aa, Artificial Sequence
  VL CDR1
QSVYNNNNLA

SEQ ID NO: 203 - protein, 11 aa, Artificial Sequence
  VL CDR2
LLIYSASSLAS

SEQ ID NO: 204 - protein, 12 aa, Artificial Sequence
  VL CDR3
AGYKTYSNNENA

SEQ ID NO: 205 - protein, 10 aa, Artificial Sequence
  VH CDR1
FSFSSSYYMC

SEQ ID NO: 206 - protein, 15 aa, Artificial Sequence
  VH CDR2
WIACIYAGSSSSTYY

SEQ ID NO: 207 - protein, 21 aa, Artificial Sequence
  VH CDR3
RAGYIDSYVDYTYAAWYYFDL

SEQ ID NO: 208 - protein, 9 aa, Artificial Sequence
  VL CDR1
ENIYSNLAW

SEQ ID NO: 209 - protein, 11 aa, Artificial Sequence
  VL CDR2
LLIYGASNLES

SEQ ID NO: 210 - protein, 11 aa, Artificial Sequence
  VL CDR3
QSADLSSSINV

SEQ ID NO: 211 - protein, 121 aa, Artificial Sequence
  VH
QSLEESGGDLVKPGPSLTLTCKASGFTISNNYWICWVRQAPGKGLEWIGCIATGDGSTYYASWAKGRFTISKTSSTTVTLQMTSLTAADTATYFCARGAAGSSWTTYFDFWGPGTPVTVSS

SEQ ID NO: 212 - protein, 111 aa, Artificial Sequence
  VL
DIVMTQTPASVEAAVGGTVTIKCQAGQNIDNYLSWYQQKPGQPPKLLIYRASTLASGVPSRFKGSGSGTEFTLTISDLECADAATYYCQNNNGGSTFTGFPFGGGTEVVVK

SEQ ID NO: 213 - protein, 119 aa, Artificial Sequence
  VH
QSLEESGGDLVKPGASLTLTCTASGFDFSSNGMCWVRQAPGKGLEWIACIYVDSSDNTNYASWVNGRFTISRTSSTTVTLQMTSLTAADTATYFCARGYGYVGSAMDLWGQGTLVTVSS

SEQ ID NO: 214 - protein, 112 aa, Artificial Sequence
  VL
DIVMTQTPASVEAAVGGTVTIKCQASQSINNELSWYQQKPGQRPKLLIYGASTLESGVPSRFSGSGSGTEFTLTISDLECADAATYYCQSAYYSSSSSYANTFGGGTEVVVK

SEQ ID NO: 215 - protein, 129 aa, Artificial Sequence
  VH
QSLEESGGDLVKPGASLTLTCKASGFTISSIWICWVRQAPGKGLEWIACIYAGSDGGSYYASWARGRFTISKTSSTTVTLQMTSLTAADTATYFCARASNSWQYGYAGYGNYKDYFNLWGPGTLVTVSS

SEQ ID NO: 216 - protein, 109 aa, Artificial Sequence
  VL
FELTQTPSSVEAVVGGTVTINCQASESISNLLAWYQQKSGQPPKVLIYKASALPSGVSSRFKGSGSGTEFTLTISDLECADAATYYCQSYYGSSDTGNTFGGGTEVVVK

SEQ ID NO: 217 - protein, 119 aa, Artificial Sequence
  VH
QSLEESGGDLVKPGASLTLTCTASGFSFSSSYWISWVRQAPGKGLEWIACIAIGSSGTTYYASWAKGRFTISKTSSTTVTLQMTSLTAADTATYFCARSGDGYTYVELWGPGTLVTVSS

SEQ ID NO: 218 - protein, 110 aa, Artificial Sequence
  VL
DVVMTQTPASVSEPVGGTVTIKCQASQSISSYLAWYQQKPGQPPKLLIYRASTLASGVPSRFKGSGSGTQFTLTISDLECADAATYYCQNYYDIDDSDNTFGGGTEVVVK

SEQ ID NO: 219 - protein, 128 aa, Artificial Sequence
  VH
QEQLEESGGDLVKPEGSLTLTCTASGFSFSWICWVRQAPGKGLEWIACIYAGSSAKTYYASWAKGRFTISKASSTTVTLQMTSLTAADTATYFCARASNYYRYGVAGYADYTGYFNLWGPGTLVTVSS

SEQ ID NO: 220 - protein, 108 aa, Artificial Sequence
  VL
FELTQTPASVEAAVGGTVTIKCQASQSISSWLAWYHQKPGQRPKLLIYGTSTLASGVPSRFKGSGSGTEFTLTISDLECADAATYYCQNYGGSSSGDAFGGGTEVVVK

SEQ ID NO: 221 - protein, 129 aa, Artificial Sequence
  VH
QSLEESGGDLVKPGASLTLTCTASGFSFSSNYWICWVRQAPGKGLEWIACIATGSSGSTYYASWAKGRFTISKTSSTTVTLQMTSLTAADTATYFCARGEYTYGYVEYAIVTQYYFDLWGPGTLVTVSS

SEQ ID NO: 222 - protein, 109 aa, Artificial Sequence
  VL
FEMTQTPSSVSAAVGGTVTINCQASQNIYSNLAWYQQKPGQRPKLLIYGASNLESGVPSRFKGSGSGTEYTLTISDLECDDAATYYCQSADYIGSAYNAFGGGTEVVVK

SEQ ID NO: 223 - protein, 119 aa, Artificial Sequence
  VH
QSLEESGGDLVKPGASLTLTCTASGFSFSSSYYMCWVRQAPGKGLEWIACIYTTYGGTWYASWAKGRFTISKTSSTTVTLQMTSLTDADTATYFCARSSISDVTYFNLWGPGTLVTVSS

SEQ ID NO: 224 - protein, 108 aa, Artificial Sequence
  VL
DIVMTQTPASVEAAVGGTVTIKCQASQSISNYLAWYQQKPGQPPELLIYRASTLESGVPSRFKGSGSGTGFTLTISDLECADAATYYCQSYYDNNNYAFGGGTEVVVK

SEQ ID NO: 225 - protein, 117 aa, Artificial Sequence
  VH
QEQLKESGGDLVKPGASLTLTCTASGFTLSTYWVCWVRQAPGKGLEWIGCIDTVSSGDTYFASWAKGRFTGSKTSSTTVTLQMTSLTAADTATYFCARRTGSGWTLWGPGTLVTVSS

SEQ ID NO: 226 - protein, 111 aa, Artificial Sequence
  VL
LVMTQTPSPVSAAVGGTVTISCQSSQSVYNNNYLAWFQQNPGQPPKLLIYWASKLASGVPSRFKGSGSGTQFTLTISDVQCDDAATYYCLGAYVSNGWYFAFGGGTEVVVK

SEQ ID NO: 227 - protein, 115 aa, Artificial Sequence
  VH
QSLEESGGDLVKPGASLTLTCTASGFSFSSYWTCWVRQAPGKGLEWLGCTDGGSSGDTYYATWAKGRVAISKTSSTTVTLQVTSLTAADTATYFCARNLITWDLWGPGTLVTVSS

SEQ ID NO: 228 - protein, 111 aa, Artificial Sequence
  VL
IVMTQTPSSKSVPVGDTVTINCQASESVYNNNQLSWFQQKPGQPPKLLIYWASKLASGVPSRFKGSGSGTQFTLTISDVVCDDAATYYCAGYKSSITDGNAFGGGTEVVVK

SEQ ID NO: 229 - protein, 128 aa, Artificial Sequence
  VH
QSLEESGGDLVQPEGSLTLTCKASGFDFSTNIMCWVRQAPGKGLEWIACIYAGDGSTYYASWVNGRFTISKTSSTTVTLQMTSLTAADTATYFCARASTYWNYGYAGYGYYPGYFNLWGPGTLVTVSS

SEQ ID NO: 230 - protein, 112 aa, Artificial Sequence
  VL
DIVMTQTPASVEAAVGGTVTIKCQASQSIISALAWYQQKPGQPPKLLIYAASTLASGVPSRFKGSGSGTQFTLTISDLECADAATYYCQTYAYSTKSNYGSVFGGGTEVVVK

SEQ ID NO: 231 - protein, 121 aa, Artificial Sequence
  VH
QQQLVESGGGLVKPGASLTLTCKASGFSFSSGYDMCWVRQAPGKGLEWIACIYTGDGSTYYASWARGRFTISKTSSTTVTLQMTSLTAADTATYFCAREDVSSGDYTFNLWGPGTLVTVSS

SEQ ID NO: 232 - protein, 109 aa, Artificial Sequence
  VL
YDMTQTPASVEVTVGGTVTIKCQASEDIYSGLAWYQQKPGQRPKLLIYKASNLASGVPSRFSGSGSGTEFTLTISGVECADAATYYCQQGVTYSNVDNTFGGGTEVVVK

SEQ ID NO: 233 - protein, 129 aa, Artificial Sequence
  VH
QSLEESGGDLVKPGASLTLTCTASGFSFSSTYYMCWVRQAPGKGLEWIACIYTGSTGSTYYASWAKGRFTGSKTSSTTVTLQMTSLTAADTATYFCARGDYTYAYAGGAHVTNYYFDLWGPGTLVTVSS

SEQ ID NO: 234 - protein, 110 aa, Artificial Sequence
  VL
LVMTQTPSSVSAAVGGTVTINCQASQNIYSNLAWYQQKPGQRPKLLIFGASNLESGVPSRFKGSGSGTEFTLTISDLECDDAATYYCQTADYSSSTDWGAFGGGTEVVVK

SEQ ID NO: 235 - protein, 119 aa, Artificial Sequence
  VH
QSLEESGGDLVKPGASLTLTCTASGFDFSSNGMCWVRQAPGKGLEWIACIYVDSSDSTYYASWVNGRFTISRTSSTTVTLQMTSLTAADTATYFCARGYGYVGSAMDLWGQGTLVTVSS

SEQ ID NO: 236 - protein, 112 aa, Artificial Sequence
  VL
DIVMTQTPASVEAAVGGTVTIKCQASQSISNELSWYQQKPGQRPKLLIYGASTLESGVPSRFSGSGSGTEFTLTISDLECADAATYYCQSAYYSSSSSYANTFGGGTEVVAA

SEQ ID NO: 237 - protein, 117 aa, Artificial Sequence; identical to SEQ ID NO: 225
  VH

SEQ ID NO: 238 - protein, 111 aa, Artificial Sequence
  VL
LVMTQTPSPVSAAVGGTVTISCQSSQSVYNNNYLAWFQQNPGQPPKLLIYWASKLASGVPSRFKGSGSGTQFTLTISDVQCDDAATYYCLGAYVSNGWYFAFGGGIEVVVK

SEQ ID NO: 239 - protein, 130 aa, Artificial Sequence
  VH
QEHLEESGGGLVKPEGSLTLTCTASGFSFSSSYYMCWVRQAPGKGLEWIACIYPDDGNTYYASWAKGRFTISKTSSTTVTLQMTSLTAADTATYFCARALAYYAYVDGGHSYAINDFDLWGPGTLVTVSS

SEQ ID NO: 240 - protein, 111 aa, Artificial Sequence
  VL
QVLTQTPSSVSAAVGGTVTINCQSSQSVFSNDYFSWYQQKPGQPPKLLIYDASRLASGVPSRFKGSGSGTQFTLTISGVQCDDAATYYCQGTYYSSAWYNAFGGGTEVVVK

SEQ ID NO: 241 - protein, 119 aa, Artificial Sequence
  VH
QSLEESGGDLVKPGASLTLTCTASGFDFSSNGMCWVRQAPGKGLEWIACIYVDSSDNTNYASWVNGRFTISRTSSTTVDLKMTSLTAADTATYFCARGYGYVGSAMDLWGQGTLVTVSS

SEQ ID NO: 242 - protein, 112 aa, Artificial Sequence
  VL
DIVMTQTPASVEAAVGGTVTIKCQASQSISNELSWYQQKPGQRPKLLIYGASTLESGVPSRFSGSGSGTEFTLTISDLECADAATYYCQSAYYSSSSSYANTFGGGTEVVVK

SEQ ID NO: 243 - protein, 118 aa, Artificial Sequence
  VH
QEQLEESGGDLVKPGGTLTLTCKASGIDFSSDYYMCWVRQAPGKGLEWIACIYTGSSDDTYYASWAKGRFTISKTSSPTVALQMTSLTAADTATYFCARGGYGGKDLWGPGTLVTVSS

SEQ ID NO: 244 - protein, 112 aa, Artificial Sequence
  VL
VVMTQTPASVEAAVGGTVTIKCQASQSIGSLLAWYQQKPGQPPNLLIYWASTLASGVPSRFKGSGSGTEFTLTISDLECDDAATYYCQCTYGSSGSSSYLNAFGGGTEVVVK

SEQ ID NO: 245 - protein, 119 aa, Artificial Sequence
  VH
QSLEESGGDLVKPGASLTLTCTASGFDFSSNGMCWVRQAPGKGLEWIACIYVDSSDSTYYASWVNGRFTISKTSSTTVTLQMTSLTAADTATYFCARGYGYVGSAMDLWGQGTLVTVSS

SEQ ID NO: 246 - protein, 112 aa, Artificial Sequence
  VL
NIVMTQTPSPVSGAVGGTVTIKCQASQSISNELAWFQQKPGQRPKLLIYGASTLESGVPSRFSGSGSGTEFTLTISDLECADAATYYCQSAYYSSSSSYANTFGGGTEVVVK

SEQ ID NO: 247 - protein, 117 aa, Artificial Sequence
  VH
QEQLKESGGDLVKPGASLTLTCTASGFTLSTYWVCWVRQAPGKGLEWIGCIDTVSSGDTYFASWAKGRFTGSKTSSTTVTLQMTSLTAADTATYFCARRTGSRWTLWGPGTLVTVSS

SEQ ID NO: 248 - protein, 111 aa, Artificial Sequence
  VL
QVLTQTPSSVSAGVGGTVTINCQASESVYNNNYLSWYQQKPGQPPKLLIYQASTLASGVPSRFKGSGSGTQFTLTISDVQCDDAATYYCLGAFVSNGWYFAFGGGTEVVVK

SEQ ID NO: 249 - protein, 130 aa, Artificial Sequence
  VH
QSLEESGGDLVKPGASLTLTCTASGFSFSSGYNICWVRQAPGKGLELIACIYTSSSGSTYYASWAKGRFTISKTSSTTVTLQMTSLTVADTATYFCARGEAYYAYGYVGYAYYHGAFDPWGPGTLVTVSS

SEQ ID NO: 250 - protein, 110 aa, Artificial Sequence
  VL
DIVMTQTPASVEAGVGGTVTIKCQASHSISKYFSWYQQKIGQPPKLLIYEASTLASGVPSRFKGSGSGTQFTLTISDLECADAATYYCQSYYYGTSSSYAFGGGTEVVVK

SEQ ID NO: 251 - protein, 126 aa, Artificial Sequence
  VH
QSLEESGGDLVKPGASLTLTCTASGFSFSSSYYMCWVRQAPGKGLEWIACIYAGSSGGTYYASWAKGRFTISKTSSTTVTLQMTSLTAADTATYFCARAFSYYYSDGYTGYAYGLWGPGTLVTVSS

SEQ ID NO: 252 - protein, 110 aa, Artificial Sequence
  VL
DIVMTQTPASVEAAVGGTVTIKCQASQSISSYLAWYQQKPGQPPKLLIYRASTLASGVPSRFKGSGSGTQFTLTISDLECADAATYYCQGAYYSSSSSYGFGGGTEVVVK

SEQ ID NO: 253 - protein, 122 aa, Artificial Sequence
  VH
QEQLVESGGGLVQPEGSLTLTCTASGFSFSGYDMCWVRQAPGKGLEWIACIHSSSGTYYANWAKGRFTISKTSSTTVTLQMTSLAADTATYFCARDFSYTDDYISYVYATDWGPGTLVTVSS

SEQ ID NO: 254 - protein, 107 aa, Artificial Sequence
  VL
AIEMTQTPASVSAAVGGTVTIKCQASQGSSLAWYQQKPGQPPKLLIYAASYLASVPSRFKGSGSGTEYTLTISGVQCADAAYYCQSTYYSSSTDIRAFGGGTEVVVK

SEQ ID NO: 255 - protein, 125 aa, Artificial Sequence
  VH
QSLEESGGDLVKPGASLTLTCTASGFSFSSYWICWVRQAPGKGLEWIACIYAGSSGTYYASWAKGRFTISKTSSTTVTLQTTSLAADTATYFCARAEYIDGYADYTYTTLYFDLWGPGTPVTVSS

SEQ ID NO: 256 - protein, 106 aa, Artificial Sequence
  VL
ALVMTQTPSSVSAAVGGTVTINCQASQIYNNLAWYQQKPGQRPKLLIYGASNLEGVPSRFKGSGSGTEYTLTISDLECDDAAYYCQSADLTSSINVFGGGTEVVVK

SEQ ID NO: 257 - protein, 120 aa, Artificial Sequence
  VH
QQQLEESGGDLVQPGASLTLTCTASGFSFNSNYWMCWGRQAPGKGLEWIGCILFGNTDTYYANWAKGRFTISKTSSTTVTLQMTSLTAADTATYFCARSVSGVGSAWNLWGPGTLVTVSS

SEQ ID NO: 258 - protein, 111 aa, Artificial Sequence
  VL
IVMTQTPSSKSVPVGDTVTINCQASESVYNNNHLAWYQQKPGQSPKLLIYLASILDSGVPSRFKGSGSGTQFTLTISDVVCDDAATYYCAGYKGITIDGSAFGGGTELVVK

SEQ ID NO: 259 - protein, 126 aa, Artificial Sequence
  VH
QSLEESGGDLVKPGASLTLTCTASGFDFSSYYWICWVRQAPGKGLEWIACIYGGSSGSTYYATWAKGRFTISETSSTTVTLQMTSLTAADMATYFCARSLYTWRYADYAASTLNLWGPGTLVTVSS

SEQ ID NO: 260 - protein, 110 aa, Artificial Sequence
  VL
AVLTQTPSPVSAAVGGTVSISCQSSQSVYNVNLLAWYQQKPGQPPKLLIYETSKLESGVPSRFSGSGSGTQFTLTISDVQCDDAATYYCAGGYSSSKDNSFGGGTEVVVK

SEQ ID NO: 261 - protein, 130 aa, Artificial Sequence
  VH
QEQLVESGGGLVQPEGSLTLTCTASGFSFSSSYFMCWVRQAPGKGLEWIACIYTGDGNNYYASWAKGRFTISKTSSTTVTLQMTSLTAADTATYFCSRGGSYYAYGYAGYDYYPDAFDYWGPGTLVTVSS

SEQ ID NO: 262 - protein, 110 aa, Artificial Sequence
  VL
DIVMTQTPASVEVAVGGTVTIKCQASQSISSYLSWYQQKPGQPPKLLIYRASTLASGVPSRFKGSGSGTQFTLTISDLECADAATYYCQSYYYSSSGSYGFGGGTEVVVK

SEQ ID NO: 263 - protein, 120 aa, Artificial Sequence
  VH
QSLEESGGDLVKPGASLTLTCTASGFSFNSYYMCWVRQAPGKGLEWIACISGGSSDNTYYASWAKGRFTTSKTSSTTVTLQMTSLTAADTATYFCARDIPRSGYFGCDLWGPGTLVTVSS

SEQ ID NO: 264 - protein, 109 aa, Artificial Sequence
  VL
VVMTQTPASVSEPVGGTVTIKCQASQNIYSNLAWYQQKPGQRPKLLIYGASNLESGVPSRFKGSGSGTEYTLTISNLECADAATYYCQSTVYNSNYANTFGGGTEVVVK

SEQ ID NO: 265 - protein, 127 aa, Artificial Sequence
  VH
QSLEESGGDLVKPGASLTLTCTASGFSFSSSYWIYWVRQAPGKGLEWIACIYTASRGSIYYASWTKGRFTISKTSSTTVTLQMTSLTAADTATYFCARGPDYTYGYIGDALTRLDLWGQGTLVTVSS

SEQ ID NO: 266 - protein, 113 aa, Artificial Sequence
  VL
AVLTQTPSPVSAAVGGTVSISCQSSQSVYDNNWLAWYQQKAGQPPKLLIYAASTLSSGVPSRFKGSGSGIEFTLTISDVQCDDAATYYCAGGYSSTSDIEDNTFGGGTEVVVK

SEQ ID NO: 267 - protein, 129 aa, Artificial Sequence
  VH
QSLEESGGDLVKPGASLTLTCTASGFSFSSSYWICWVRQAPGKGLEWIACIYAGSSGDTYYASWAKGRFTISKTSSTTVTLQTTSLTAADTATYFCARAEYIDGYADYTYTTLYYFDLWGPGTPVTVSS

SEQ ID NO: 268 - protein, 108 aa, Artificial Sequence
  VL
DVVMTQTPASVSEPVGGTVTINCQASESINSWLAWYQQKPGQPPKLLIYSASTLASGVPSRFKGSGSGIEFTLTISDLECADAATYFCQSYYSFSRFAFGGGTEVVVK

SEQ ID NO: 269 - protein, 120 aa, Artificial Sequence
  VH
QSLEESGGDLVKPGASLTLTCTASGFSFSSGYWICWVRQAPGKGLEWIACIYTGVGATYYASWAKGRFTISKTSSTTVTLQMTSLTAADTATYFCARDFGGSSGFYFNLWGPGTLVTVSS

SEQ ID NO: 270 - protein, 111 aa, Artificial Sequence
  VL
FELTQTPSSVEAAVGATVTIKCQASQSISNALAWYQQKPGQPPKLLIYSASTLESGVPSRFKGSGSGTEFTLTISDLECADAATYYCQNYYGSTSSSYGVAFGGGTEVVVK

SEQ ID NO: 271 - protein, 128 aa, Artificial Sequence
  VH
QSLEESGGGLVQPEGSLTLTCTASGFSFSSSYYMCWVRQAPGKGLEWIACIYAGSTFSTYYASWAKGRFTISKTSSTTVTLQMTSLTAADTATYFCARSDSYYTYGYAGYAYAIFNLWGPGTLVTVSS

SEQ ID NO: 272 - protein, 109 aa, Artificial Sequence
  VL
LVMTQTPSSVSAAVGGTVTINCQASESIYSNLAWYQQKPGQPPKLLIYLASTLASGVPSRFKGSGSGTEFTLTISDLECADAATYYCQSAYYSSSADIAFGGGTEVVVK

SEQ ID NO: 273 - protein, 119 aa, Artificial Sequence
  VH
QSLEESGGDLVKPGASLTLTCTASGLDFSSSYWICWVRQAPGKGLEWIGCIKTATETTVYASWAKGRFTISKTSSTTVTLQMTSLTAADTATYLCAKTYADNGGYINLWGPGTLVTVSS

SEQ ID NO: 274 - protein, 108 aa, Artificial Sequence
  VL
LVMTQTPSSVSAAVGGTVTINCQASQNIYNNLAWYQQKPGQRPKLLIYGASNLESGVPSRFKGSGSGTEYTLTISDLECDDAATYYCQSADLTSSINVFGGGTEVVVK

SEQ ID NO: 275 - protein, 127 aa, Artificial Sequence
  VH
QSLEESGGDLVKPGASLTLTCTASGFSFSSSYWICWVRQAPGKGLEWIACIYTASRDSIYYASWAKGRFTISKTSSTTVTLQMTSLTAADTATYFCARGPYYSYAYIGDALTRLDLWGQGTLVTVSS

SEQ ID NO: 276 - protein, 113 aa, Artificial Sequence
  VL
AVLTQTPSPVSAAVGGTVSISCQSSQSVYDNNWLAWYQQKPGQPPKLLIYEASKLASGVPSRFKGSGSGTQFTLTISGVQCDDASTYYCAGGYSSSSDIEDNTFGGGTEVVVK

SEQ ID NO: 277 - protein, 122 aa, Artificial Sequence
  VH
QEQLEESGGDLVKPGASLTLTCTASGFSFNSNYYMCWVRQAPGKGLEWIACIYTGIVVPTYYASWAKGRFTISKTSSTTVTLQMTSLTAADTATYFCARDPYVGSSYIYNLWGPGTLVTVSS

SEQ ID NO: 278 - protein, 111 aa, Artificial Sequence
  VL
LVMTQTPSPVSAAVGSTVTISCQSSQSVYNNNNLAWYQQKPGQPPKLLIYSASSLASGVPSRFKGSGSGTQFTLTISGVECDDAATYYCAGYKTYSNNENAFGGGTEVVVK

SEQ ID NO: 279 - protein, 129 aa, Artificial Sequence
  VH
QSLEESGGDLVKPGASLTLTCTASGFSFSSSYYMCWVRQAPGKGLEWIACIYAGSSSSTYYASWAKGRFTISKTSSTTVTLQTTSLTAADTATYFCARAGYIDSYVDYTYAAWYYFDLWGPGTLVTVSS

SEQ ID NO: 280 - protein, 111 aa, Artificial Sequence
  VL
QVLTQTPSSVSEPVGGTVTINCQASENIYSNLAWYHQKPGQRPKLLIYGASNLESGVPSRFKGSGSGTEYTLYHQTISDLECDDAATYYCQSADLSSSINVFGGGTEVVVK

SEQ ID NO: 281 - protein, 123 aa, Artificial Sequence
  VH
EVQLVESGGGLVQPGGSLRLSCAASGFTISNNYWICWVRQAPGKGLEWIGCIATGDGSTYYASWAKGRFTISRDNSKNTLYLQMNSLRAEDTAVYYCARGAAGSSWTTYFDFWGQGTLVTVSS

SEQ ID NO: 282 - protein, 111 aa, Artificial Sequence
  VL
DIQMTQSPSSLSASVGDRVTITCQAGQNIDNYLSWYQQKPGKVPKLLIYRASTLASGVPSRFSGSGSGTDFTLTISSLQPEDVATYYCQNNNGGSTFTGFPFGQGTKVEIK

SEQ ID NO: 283 - protein, 121 aa, Artificial Sequence
  VH
EVQLVESGGGLVQPGGSLRLSCAASGFSFSSSYWISWVRQAPGKGLEWIACIAIGSSGTTYYASWAKGRFTISRDNSKNTLYLQMNSLRAEDTAVYYCARSGDGYTYVELWGQGTLVTVSS

SEQ ID NO: 284 - protein, 110 aa, Artificial Sequence
  VL
DIQMTQSPSSLSASVGDRVTITCQASQSISSYLAWYQQKPGKVPKLLIYRASTLASGVPSRFSGSGSGTDFTLTISSLQPEDVATYYCQNYYDIDDSDNTFGQGTKVEIK

SEQ ID NO: 285 - protein, 121 aa, Artificial Sequence
  VH
EVQLVESGGGLVQPGGSLRLSCAASGFSFSSSYYMCWVRQAPGKGLEWIACIYTTYGGTWYASWAKGRFTISRDNSKNTLYLQMNSLRAEDTAVYYCARSSISDVTYFNLWGQGTLVTVSS

SEQ ID NO: 286 - protein, 108 aa, Artificial Sequence
  VL
DIQMTQSPSSLSASVGDRVTITCQASQSISNYLAWYQQKPGKVPKLLIYRASTLESGVPSRFSGSGSGTDFTLTISSLQPEDVATYYCQSYYDNNNYAFGQGTKVEIK

SEQ ID NO: 287 - protein, 117 aa, Artificial Sequence
  VH
EVQLVESGGGLVQPGGSLRLSCAASGFSFSSYWTCWVRQAPGKGLEWLGCTDGGSSGDTYYATWAKGRFTISRDNSKNTLYLQMNSLRAEDTAVYYCARNLITWDLWGQGTLVTVSS

SEQ ID NO: 288 - protein, 112 aa, Artificial Sequence
  VL
DIQMTQSPSSLSASVGDRVTITCQASESVYNNNQLSWYQQKPGKVPKLLIYWASKLASGVPSRFSGSGSGTDFTLTISSLQPEDVATYYCAGYKSSITDGNAFGQGTKVEIK

SEQ ID NO: 289 - protein, 121 aa, Artificial Sequence
  VH
EVQLVESGGGLVQPGGSLRLSCAASGFDFSSNGMCWVRQAPGKGLEWIACIYVDSSDNTNYASWVNGRFTISRDNSKNTLYLQMNSLRAEDTAVYYCARGYGYVGSAMDLWGQGTLVTVSS

SEQ ID NO: 290 - protein, 112 aa, Artificial Sequence
  VL
DIQMTQSPSSLSASVGDRVTITCQASQSISNELSWYQQKPGKVPKLLIYGASTLESGVPSRFSGSGSGTDFTLTISSLQPEDVATYYCQSAYYSSSSSYANTFGQGTKVEIK

SEQ ID NO: 291 - protein, 132 aa, Artificial Sequence
  VH
EVQLVESGGGLVQPGGSLRLSCAASGFSFSSGYNICWVRQAPGKGLELIACIYTSSSGSTYYASWAKGRFTISRDNSKNTLYLQMNSLRAEDTAVYYCARGEAYYAYGYVGYAYYHGAFDPWGQGTLVTVSS

SEQ ID NO: 292 - protein, 110 aa, Artificial Sequence
  VL
DIQMTQSPSSLSASVGDRVTITCQASHSISKYFSWYQQKPGKVPKLLIYEASTLASGVPSRFSGSGSGTDFTLTISSLQPEDVATYYCQSYYYGTSSSYAFGQGTKVEIK

SEQ ID NO: 293 - protein, 130 aa, Artificial Sequence
  VH
EVQLVESGGGLVQPGGSLRLSCAASGFSFSSSYYMCWVRQAPGKGLEWIACIYAGSTFSTYYASWAKGRFTISRDNSKNTLYLQMNSLRAEDTAVYYCARSDSYYTYGYAGYAYAIFNLWGQGTLVTVSS

SEQ ID NO: 294 - protein, 110 aa, Artificial Sequence
  VL
DIQMTQSPSSLSASVGDRVTITCQASESIYSNLAWYQQKPGKVPKLLIYLASTLASGVPSRFSGSGSGTDFTLTISSLQPEDVATYYCQSAYYSSSADIAFGQGTKVEIK

SEQ ID NO: 295 - protein, 26 aa, Artificial Sequence
  VHFR1
EVQLVESGGGLVQPGGSLRLSCAASG

SEQ ID NO: 296 - protein, 11 aa, Artificial Sequence
  VHFR2
WVRQAPGKGLE

SEQ ID NO: 297 - protein, 37 aa, Artificial Sequence
  VHFR3
ASWAKGRFTISRDNSKNTLYLQMNSLRAEDTAVYYCA

SEQ ID NO: 298 - protein, 11 aa, Artificial Sequence
  VHFR4
WGQGTLVTVSS

SEQ ID NO: 299 - protein, 26 aa, Artificial Sequence
  VLFR1
DIQMTQSPSSLSASVGDRVTITCQAG

SEQ ID NO: 300 - protein, 11 aa, Artificial Sequence
  VLFR2
WYQQKPGKVPK

SEQ ID NO: 301 - protein, 32 aa, Artificial Sequence
  VLFR3
GVPSRFSGSGSGTDFTLTISSLQPEDVATYYC

SEQ ID NO: 302 - protein, 10 aa, Artificial Sequence
  VLFR4
FGQGTKVEIK

SEQ ID NO: 303 - protein, 10 aa, Artificial Sequence
  VH CDR1
FSFSSSYYMC

SEQ ID NO: 304 - protein, 14 aa, Artificial Sequence
  VH CDR2
WIGCIYTGNDDTWY

SEQ ID NO: 305 - protein, 8 aa, Artificial Sequence
  VH CDR3
RGLSPIDL

SEQ ID NO: 306 - protein, 10 aa, Artificial Sequence
  VL CDR1
ESIYNNNNLG

SEQ ID NO: 307 - protein, 11 aa, Artificial Sequence
  VL CDR2
LLIYWASTLAS

SEQ ID NO: 308 - protein, 13 aa, Artificial Sequence
  VL CDR3
AGYKSRTTDGSAF

SEQ ID NO: 309 - protein, 10 aa, Artificial Sequence
  VH CDR1
FSFSSGYDMC

SEQ ID NO: 310 - protein, 14 aa, Artificial Sequence
  VH CDR2
WIACIHSSSGTTYY

SEQ ID NO: 311 - protein, 18 aa, Artificial Sequence
  VH CDR3
RDFSYTDDYISYVYATDL

SEQ ID NO: 312 - protein, 8 aa, Artificial Sequence
  VL CDR1
QSIGSSLA

SEQ ID NO: 313 - protein, 11 aa, Artificial Sequence
  VL CDR2
LLIYAASYLAS

SEQ ID NO: 314 - protein, 13 aa, Artificial Sequence
  VL CDR3
QSTYYSSSTDIRA

SEQ ID NO: 315 - protein, 116 aa, Artificial Sequence
  VH
QERLEESGGGLVQPEGSLTLTCTASGFSFSSSYYMCWVRQAPGKGMEWIGCIYTGNDDTWYASWAKGRFTVSKTSSTTVTLQMTSLTATDTATYFCARGLSPIDLWGPGTLVTVSS

SEQ ID NO: 316 - protein, 111 aa, Artificial Sequence
  VL
IVMTQTPSSKSVPVGDTVTINCQASESIYNNNNLGWYQQKPGQPPKLLIYWASTLASGVPSRFKGSGSGTQFTLTISDVECDDAATYYCAGYKSRTTDGSAFGGGTEVVVK

SEQ ID NO: 317 - protein, 127 aa, Artificial Sequence
  VH
EVQLVESGGGLVQPGGSLRLSCAASGFSFSSGYDMCWVRQAPGKGLEWIACIHSSSGTTYYASWAKGRFTISRDNSKNTLYLQMNSLRAEDTAVYYCARDFSYTDDYISYVYATDLWGQGTLVTVSS

SEQ ID NO: 318 - protein, 111 aa, Artificial Sequence
  VL
DIQMTQSPSSLSASVGDRVTITCQASQSIGSSLAWYQQKPGKVPKLLIYAASYLASGVPSRFSGSGSGTDFTLTISSLQPEDVATYYCQSTYYSSSTDIRAFGQGTKVEIK

SEQ ID NO: 319 - protein, 97 aa, Artificial Sequence
  CH1 domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKK

SEQ ID NO: 320 - protein, 107 aa, Artificial Sequence [sequence omitted]

SEQ ID NO: 321 - protein, 227 aa, Artificial Sequence
  WT CH2CH3
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 322 - protein, 227 aa, Artificial Sequence
  FC1 CH2CH3
DKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 323 - protein, 227 aa, Artificial Sequence
  FC2 CH2CH3
DKTHTCPPCPAPELLGGPSVFLLPPKPKDTLMISRTPEVTCVVVDVSHEDPEVKFNWYVDGVEVHNAKTKPPEEQYNSTLRVVSILTVLHQDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEALHNHYTQKSLSLSPGK

SEQ ID NO: 324 - protein, 120 aa, Artificial Sequence
  VH of comparator antibody
QVQLQQSGGGLVKPGGSLRLSCAASGFTFSSYDMHWVRQAPGKGLEWVAVISYDGSNKYYADSVKGRFTISRDNSKNTLYLQMDSLRAEDTAVYFCAKEGDSSRWSYDLWGRGTLVTVSS

SEQ ID NO: 325 - protein, 112 aa, Artificial Sequence
  VL of comparator antibody
QSALTQPASVSGSPGQSITISCTGTSSDVGGYNYVSWYQQHPGKAPKVMIYDVTNRPSGVSNRFSGSKSGNTASLTISGLQAEDEADYYCSSYTSTSTLVVFGGGTKLTVLG

SEQ ID NO: 326 - protein, 456 aa, Artificial Sequence
  Humanized C36 heavy chain with FC-1 mutations
EVQLVESGGGLVQPGGSLRLSCAASGFSFSSGYNICWVRQAPGKGLELIACIYTSSSGSTYYASWAKGRFTISRDNSKNTLYLQMNSLRAEDTAVYYCARGEAYYAYGYVGYAYYHGAFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 327 - protein, 456 aa, Artificial Sequence
  Humanized C36 heavy chain with FC-2 mutations
EVQLVESGGGLVQPGGSLRLSCAASGFSFSSGYNICWVRQAPGKGLELIACIYTSSSGSTYYASWAKGRFTISRDNSKNTLYLQMNSLRAEDTAVYYCARGEAYYAYGYVGYAYYHGAFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKDKTHTCPPCPAPELLGGPSVFLLPPKPKDTLMISRTPEVTCVVVDVSHEDPEVKFNWYVDGVEVHNAKTKPPEEQYNSTLRVVSILTVLHQDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEALHNHYTQKSLSLSPGK

SEQ ID NO: 328 - protein, 217 aa, Artificial Sequence
  Humanized C36 light chain
DIQMTQSPSSLSASVGDRVTITCQASHSISKYFSWYQQKPGKVPKLLIYEASTLASGVPSRFSGSGSGTDFTLTISSLQPEDVATYYCQSYYYGTSSSYAFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 329 - protein, 447 aa, Artificial Sequence
  Humanized C4 heavy chain with FC-1 mutations
EVQLVESGGGLVQPGGSLRLSCAASGFTISNNYWICWVRQAPGKGLEWIGCIATGDGSTYYASWAKGRFTISRDNSKNTLYLQMNSLRAEDTAVYYCARGAAGSSWTTYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 330 - protein, 447 aa, Artificial Sequence
  Humanized C4 heavy chain with FC-2 mutations
EVQLVESGGGLVQPGGSLRLSCAASGFTISNNYWICWVRQAPGKGLEWIGCIATGDGSTYYASWAKGRFTISRDNSKNTLYLQMNSLRAEDTAVYYCARGAAGSSWTTYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKDKTHTCPPCPAPELLGGPSVFLLPPKPKDTLMISRTPEVTCVVVDVSHEDPEVKFNWYVDGVEVHNAKTKPPEEQYNSTLRVVSILTVLHQDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEALHNHYTQKSLSLSPGK

SEQ ID NO: 331 - protein, 217 aa, Artificial Sequence
  Humanized C4 light chain
DIQMTQSPSSLSASVGDRVTITCQAGQNIDNYLSWYQQKPGKVPKLLIYRASTLASGVPSRFSGSGSGTDFTLTISSLQPEDVATYYCQNNNGGSTFTGFPFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 332 - protein, 451 aa, Artificial Sequence
  Humanized C53 with FC-1 mutations
EVQLVESGGGLVQPGGSLRLSCAASGFSFSSGYDMCWVRQAPGKGLEWIACIHSSSGTTYYASWAKGRFTISRDNSKNTLYLQMNSLRAEDTAVYYCARDFSYTDDYISYVYATDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 333 - protein, 451 aa, Artificial Sequence
  Humanized C53 with FC-2 mutations
EVQLVESGGGLVQPGGSLRLSCAASGFSFSSGYDMCWVRQAPGKGLEWIACIHSSSGTTYYASWAKGRFTISRDNSKNTLYLQMNSLRAEDTAVYYCARDFSYTDDYISYVYATDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKDKTHTCPPCPAPELLGGPSVFLLPPKPKDTLMISRTPEVTCVVVDVSHEDPEVKFNWYVDGVEVHNAKTKPPEEQYNSTLRVVSILTVLHQDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEALHNHYTQKSLSLSPGK

SEQ ID NO: 334 - protein, 217 aa, Artificial Sequence
  Humanized C53 light chain
DIQMTQSPSSLSASVGDRVTITCQASQSIGSSLAWYQQKPGKVPKLLIYAASYLASGVPSRFSGSGSGTDFTLTISSLQPEDVATYYCQSTYYSSSTDIRAFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 335 - protein, 5 aa, Artificial Sequence
  CD3 VH CDR1
GYGMH

SEQ ID NO: 336 - protein, 17 aa, Artificial Sequence
  CD3 VH CDR2
VIWYDGSKKYYVDSVKG

SEQ ID NO: 337 - protein, 9 aa, Artificial Sequence
  CD3 VH CDR3
QMGYWHFDL

SEQ ID NO: 338 - protein, 11 aa, Artificial Sequence
  CD3 VL CDR1
RASQSVSSYLA

SEQ ID NO: 339 - protein, 7 aa, Artificial Sequence
  CD3 VL CDR2
DASNRAT

SEQ ID NO: 340 - protein, 10 aa, Artificial Sequence
  CD3 VL CDR3
QQRSNWPPLT

SEQ ID NO: 341 - protein, 8 aa, Artificial Sequence
  CDR VH CDR1
GYTFTRYT

SEQ ID NO: 342 - protein, 8 aa, Artificial Sequence
  CD3 VH CDR2
INPSRGYT

SEQ ID NO: 343 - protein, 12 aa, Artificial Sequence
  CD3 VH CDR3
ARYYDDHYCLDY

SEQ ID NO: 344 - protein, 5 aa, Artificial Sequence
  CD3 VL CDR1
SSVSY

SEQ ID NO: 345 - protein, 3 aa, Artificial Sequence
  CD3 VL CDR2
DTS

SEQ ID NO: 346 - protein, 7 aa, Artificial Sequence
  CD3 VL CDR3
QQWSSNP

(19) United States
(12) Patent Application Publication
(10) Pub. No.: US 2023/0080224 A1
(43) Pub. Date: Mar. 16, 2023

(54) ANTIGEN-BINDING MOLECULES AGAINST ALPPL2 AND/OR ALPP AND USES THEREOF

(71) Applicant: Agency for Science, Technology and Research, Singapore (SG)

(72) Inventors: William SUN, Singapore (SG); Boon Ooi Patrick TAN, Singapore (SG); Huajing WANG, Singapore (SG); Thai Leong YAP, Singapore (SG); Shin Yee HONG, Singapore (SG); Cheng-I WANG, Singapore (SG); Ching-Wen HUANG, Singapore (SG); Shuet Theng LEE, Singapore (SG); Kah Fei WAN, Singapore (SG); Jian Duan Johnathan NG, Singapore (SG)

(73) Assignee: Agency for Science, Technology and Research, Singapore (SG)

(21) Appl. No.: 17/798,025

(22) PCT Filed: Feb. 5, 2021
(86) PCT No.: PCT/SG2021/050061

§ 371 Date: Aug. 5, 2022

(30) Foreign Application Priority Data
Feb. 7, 2020 (SG) 10202001139U

(51) Int. Cl. C07K 16/40; G01N 33/573; G01N 33/574; A61P 35/00

(52) U.S. Cl. CPC C07K 16/40; G01N 33/573; G01N 33/57446; A61P 35/00; A61K 2039/505

(57) ABSTRACT

The invention relates to antigen-binding molecules that specifically binds ALPPL2 and/or ALPP but not ALPL or ALPI. It also relates to a pharmaceutical composition, an immunoconjugate and a chimeric antigen receptor comprising said antigen-binding molecules. The invention also relates to methods for reducing the expression or activity of ALPPL2 in a cancer cell and methods of treating a cancer in a subject.

### FIELD OF INVENTION

The invention relates generally to the field of oncology. In particular, the invention relates to antigen-binding molecules that specifically binds ALPPL2 and/or ALPP but not ALPL or ALPI.

### BACKGROUND

Antibodies are attractive therapeutic agents due to their ability to bind to cell surface antigens and eliminate cancer cells. Clinically-approved antibody therapeutics include Herceptin and Rituxan, which are highly successfully drugs for treating various cancers, including blood and solid cancers. Antibody therapies work by, for example, recruiting effector cells (such as natural killer cells or T-cells) or by modulating the signalling pathway of a cancer cells. The antibodies may also be conjugated to toxins or radioisotopes to help eliminate cancer cells. The development of a successful antibody therapy requires targeting of cell surface antigens that are preferentially expressed on cancer cells. This is because the expression of the same surface antigen on normal healthy cells may lead to undesired side effects.

There is generally a lack of suitable tumor-associated antigens for targeted antibody therapy against cancer. Furthermore, it is a significant challenge to develop an effective therapy against such antigens to treat cancer.

Accordingly, it is generally desirable to overcome or ameliorate one or more of the above mentioned difficulties.

### SUMMARY

Disclosed herein is an antigen-binding molecule that specifically binds ALPPL2 and/or ALPP but not ALPL or ALPI, comprising:
(a) a heavy chain variable region (V_{H}) comprising VHCDR1, VHCDR2 and VHCDR3 amino acid sequences; and
(b) a light chain variable region (V_{L}) comprising VLCDR1, VLCDR2 and VLCDR3 amino acid sequences; wherein the combination of VHCDR1, VHCDR2, VHCDR3, VLCDR1, VLCDR2 and VLCDR3 amino acid sequences are shown in any of the rows in Table 1.

Disclosed herein is a chimeric molecule comprising an antigen-binding molecule as defined herein and a heterologous moiety.

Disclosed herein is an isolated polynucleotide comprising a nucleic acid sequence encoding the antigen-binding molecule or the chimeric molecule as defined herein.

Disclosed herein is a construct comprising a nucleic acid sequence encoding the antigen-binding molecule or the chimeric molecule as defined herein in operable connection with one or more control sequences.

Disclosed herein is a host cell that contains the construct as defined herein.

Disclosed herein is a pharmaceutical composition comprising the antigen-binding molecule or the chimeric molecule as defined herein, and a pharmaceutically acceptable carrier.

Disclosed herein is a method for reducing the expression or activity of ALPPL2 in a cancer cell, the method comprising contacting the cancer cell with an antigen-binding molecule or a chimeric molecule as defined herein.

Disclosed herein is a method for reducing or inhibiting proliferation, survival and viability of a tumor in a subject, the method comprising administering an antigen-binding molecule or a chimeric molecule as defined herein to the subject.

Disclosed herein is a method of treating cancer in a subject, wherein the method comprises administering an antigen-binding molecule or a chimeric molecule as defined herein to the subject.

Disclosed herein is an antigen-binding molecule or a chimeric molecule as defined herein for use in the treatment of cancer.

Disclosed herein is the use of an antigen-binding molecule or a chimeric molecule as defined herein in the manufacture of a medicament for the treatment of cancer.

Disclosed herein is a method of treating a disease or condition associated with the undesired expression of ALPPL2 in a subject, wherein the method comprises administering an antigen-binding molecule or a chimeric molecule as defined herein to the subject.

Disclosed herein is a kit for detecting cancer, the kit comprising an antigen-binding molecule or a chimeric molecule as defined herein.

Disclosed herein is a method of determining the likelihood of a cancer in a subject, wherein the method comprises detecting ALPPL2 in a sample obtained from the subject, wherein an increased level of ALPPL2 in the sample as compared to a reference indicates the likelihood of cancer in the subject.

Disclosed herein is a method of treating a cancer in a subject, wherein the method comprises a) detecting ALPPL2 in a sample obtained from the subject, wherein an increased level of ALPPL2 in the sample as compared to a reference indicates an increased likelihood of cancer in the subject; and b) treating a subject found to have an increased likelihood of cancer.

Disclosed herein is a method of identifying a subject who is likely to be responsive to treatment with an anti-ALPPL2 antibody, the method comprising detecting ALPPL2 in a sample obtained from the subject, wherein an increased level of ALPPL2 indicates that the subject is likely to be responsive to treatment with the ALPPL2 antibody.

Disclosed herein is a method of identifying and treating a subject who is likely to be responsive to treatment with an anti-ALPPL2 antibody, the method comprising a) detecting ALPPL2 in a sample obtained from the subject, wherein an increased level of ALPPL2 indicates that the subject is likely to be responsive to treatment with the ALPPL2 antibody; and b) treating the subject found likely to be responsive to treatment with the ALPPL2 antibody.

Disclosed herein is a method for preparing an antigen-binding molecule that specifically binds ALPPL2 but not ALPL or ALPI, the method comprising:
a) immunizing an animal, preferentially a rabbit, with ALPPL2,
b) isolating from the animal a B-cell that binds specifically to ALPPL2 but not ALPL or ALPI, and
c) determining the amino acid sequence of the antibody that is expressed by the B-cell.

### BRIEF DESCRIPTION OF DRAWINGS

Embodiments of the present invention are hereafter described, by way of non-limiting example only, with reference to the accompanying drawings in which:

FIG. 1 shows that a list of criteria was set to select for candidate genes encoding cell membrane proteins. Placental-like alkaline phosphatase, ALPPL2 emerged as one of the top candidates for subsequent target validation.

FIG. 2 shows the immunohistochemical staining of gastric cancer cell lines (top) and gastric tumour microarrays (bottom).

FIG. 3 shows identification of ALPPL2/ALPP specific clones produced from rabbit B cells supernatant by ELISA and FACS (top), and affinity measurements of selected clones in single concentration by biolayer interferometry (bottom).

FIG. 4 shows the comparison between ALPPL2 reactivity and ALPI reactivity of our humanized antibody and a comparable humanized antibody disclosed in prior art, as measured by ELISA (top) and surface plasmon resonance (bottom).

FIG. 5 shows the IHC stains of formalin fixed, paraffin embedded (FFPE) sections by C36, C45 and C130 of different gastric cancer cell lines (A). IHC stains of FFPE—human gastric, ovarian, colorectal, pancreatic, testicular, mesothelioma and endometrial tumors microarrays by C36 with different H-score of IHC 1+, IHC 2+ and IHC 3+(B). IHC stains of FFPE-normal tissues by C36. All normal tissues shows no C36 staining (negative) except placenta tissues that shows positive IHC2+ staining (C).

FIG. 6 shows the cross-reactivity of select clones produced from rabbit B cells supernatant to rhesus macaque ortholog identified through FACS screen (A). Recombinant humanized C4 and C36 clones binds to CHO cells over-expressed rhesus macaque ortholog but not WT CHO by FACS analysis (B).

FIG. 7 shows retention of high ALPP/ALPPL2 affinity after humanization of select clones, as measured by surface plasmon resonance (SPR).

FIG. 8 shows ADCC induction by humanized clones. ADCC induction as measured in coculture of high-expressing gastric cancer cell line MKN1 with Jurkats CD16A reporter cells (top row, left), and coculture of low-expressing gastric cancer cell line MKN74 with Jurkats CD16A reporter cells (top row, right). Potentiation of ADCC by C4 as measured by CellTiter-Glo assay of gastric cancer cell lines in coculture with primary NK cells (middle row, left), and coculture of ovarian and pancreatic cancer cell lines with Jurkats CD16A reporter cells (middle row, right). ADCC enhancement by Fc engineering of humanized C4 as measured in coculture of MKN74 with Jurkats CD16A reporter cells (bottom row).

FIG. 9 shows ADC killing of gastric cancer cell lines as measured by CellTiter-Glo assay. Killing of high-expressing gastric cancer cell lines by humanized clones via vc-MMAF conjugated to the secondary antibody (top row). Killing of gastric cancer cell lines by humanized C4 (middle row, left) and C12 (middle row, right) conjugated with vc-MMAE. Killing of gastric cancer cell lines by humanized C4 (bottom row, left) and C12 (bottom row, right) via vc-MMAF conjugated to the secondary antibody.

FIG. 10 shows potent killing of different cancer cell lines by T-cell engagers derived from the humanized clones, as measured by xCELLigence real-time cell analysis of the cancer cells in coculture with expanded human T-cells. C4 consistently demonstrated pM killing of gastric, ovarian and pancreatic cancer cell lines, regardless of target expression level.

FIG. 11 shows potent killing of different cancer cell lines by T-cell engagers with different anti-CD3 variants, in different formats. Humanized Fab fragment is amenable to the production of potent T-cell engagers by different anti-CD3 pairings and in different formats.

FIG. 12 shows reactivity of select clones to ALPPL2 but not ALPP and cancer cells killing potency by these clones after humanisation. FACS shows chimerized C53 and C78 bind ALPPL2 but not ALPP, whereas rabbit C4 binds both (A). ELISA shows chimerized C53 and C78 bind ALPPL2 but not ALPP (B). ADCC induction by chimerised clones as measured by co-culture of MKN74 with Jurkats CD16A reporter cells is shown (C). ELISA shows chimerized C53 and C78 cross-reacted to CHO cells overexpressing rhesus macaque ortholog identified through FACS (D). Humanized C53 shows the ALPPL2 specificity by ELISA (E) and co-culture of N87 with Jurkats CD16A reporter cells show that ADCC induction activity (F) is maintained after humanization. Humanized C53 demonstrated nM binding affinity towards ALPPL, but not ALPP determined by Biolayer Interferometry, whereas humanized C36 demonstrated similar binding affinity towards ALPPL2 and ALPP (G). Humanized C53 shows potent killing in N87 cells by T-cell engagers, as measured by xCELLigence real-time cell analysis of the N87 gastric cancer cells co-cultured with expanded human T-cells (H). Humanized C53 T-cell engager demonstrated similar pM killing as C36 T-cell engager.

FIG. 13 shows binding profile of humanized C4, C36 and C53 to different isoform of human alkaline phosphatase family, cancer cell lines and normal immune cells. Humanized C4 and C36 show binding to human ALPPL2 and ALPP, but not to human ALPI and ALPL, whereas humanized C53 binds specifically to human ALPPL2, but not to human ALPP, ALPI and ALPL transiently expressed in 293T cells by FACS analysis (A). Different doses of humanized C36, C4 and C53 show binding to ALPPL2/ALPP positive cell line (NCI-N87) but not to negative cells line (MIAPaca-2). Humanized C53 has comparable binding affinity as commercial antibody (catalogue number: eBioScience #14-9870-82), and weaker binding affinity as compared to humanized C4 and C36 (B). Humanized C36, C4 and C53 show no binding to the naïve and CD3/CD28 beads+IL-2 activated human PBMCs (CD4+/CD8+ T cell, B cell, CD11b+ myeloid Mp cells) (C). Humanized C36, C4 and C53 shows no binding to the T cell, B cells and myeloid Mp cells (D).

### DETAILED DESCRIPTION

The present disclosure teaches antigen-binding molecules that specifically binds ALPPL2 and/or ALPP, but not ALPL or ALPL The antigen-binding molecules may bind to ALPPL2 and/or ALPP or a cell expressing ALPPL2 and/or ALPP with an affinity of between about 14 pm to about 10 nM.

Disclosed herein is an antigen-binding molecule that specifically binds ALPPL2 and/or but not ALPL or ALPI, comprising: (a) a heavy chain variable region (V_{H}) comprising VHCDR1, VHCDR2 and VHCDR3 amino acid sequences; and (b) a light chain variable region (V_{L}) comprising VLCDR1, VLCDR2 and VLCDR3 amino acid sequences; wherein the combination of VHCDR1, VHCDR2, VHCDR3, VLCDR1, VLCDR2 and VLCDR3 amino acid sequences are shown in any of the rows in Table 1.

Alkaline phosphatase, placental-like 2 (ALPPL2) is a member of the alkaline phosphatase (AP) family, consisting of two closely related isoforms expressed in placental trophoblasts (ALPPL2 and ALPP), and two widely expressed members ALPL (tissue-nonspecific, liver/bone/kidney) and ALPI (intestinal). In one embodiment, the antigen-binding molecule specifically binds to ALPPL2 and/or ALPP. In one embodiment, the antigen-binding molecule specifically binds to human ALPPL2 and/or human ALPP. In one embodiment, the antigen-binding molecules have enhanced efficacy due to the high affinity towards ALPPL2 and/or ALPP.

In one embodiment, the antigen-binding molecule does not have any detectable binding to ALPL or ALPI. In one embodiment, the antigen-binding molecule does not have any detectable binding to human ALPL or human ALPI. This may also be referred to as having a dissociation constant (Kd) of more than 10 nM, more than 100 nM, more than 1 μM, more than 10 μM, more than 100 μM or more than 1 mM detectable binding to human ALPL or human ALPI. In one embodiment, the antigen-binding molecules have desirable therapeutic windows due to the lack of binding to ALPL or ALPI. In one embodiment, the antigen-binding molecules do not induce (or induce minimal) T-cell killing of normal cells.

Without being bound by theory, the inventors have isolated monoclonal antibodies against tumor-associated antigens, human placental-like alkaline phosphatases (ALPPL2) with high affinity (sub-nM Kd) and specificity (non-reactive to the closely related ALPL or ALPI), immunohistochemical activity (useful for develop of companion diagnostics), cross-reactivity to non-human primate ortholog (useful for toxicology studies). The inventors have humanized some clones by grafting the complementarity determining region (CDR) to a human IgG1 framework and showed these humanized antibodies retain high affinity to ALPPL2. The inventors have also shown that these naked humanized antibodies induced potent antibody-dependent cell cytotoxicity (ADCC) in coculture assay of Jurkats reporter and primary natural killer (NK) cells with gastric cancer cell lines. ADCC induction was also seen with ovarian and pancreatic cancer cell lines. The inventors have showed suitability of using these humanized antibodies as antibody-drug conjugates through cancer cell killing by primary conjugates and in a secondary assay. The inventors have also generated bispecific antibodies by heterodimerisation of these humanized antibodies with anti-CD3 antibodies. These bispecific antibodies functioned as potent T-cell engagers (TcE) and that achieved picomolar (pM) killing of gastric, ovarian and pancreatic cancer cell lines. Thus, these antibodies can be used as targeted therapy against tumors expressing ALPPL2 on the cell surface.

Table 1 shows the possible combinations of CDRs that can be present on an antigen-binding molecule.

| | V_{H} | V_{L} |
|---|---|---|
| CDR1 | FTISNNYWIC (SEQ ID NQ 1) | QNIDNYLS (SEQ ID NO: 4) |
| CDR2 | WIGCIATGDGSTYY (SEQ ID NO: 2) | LLIYRASTLAS (SEQ ID NO: 5) |
| CDR3 | RGAAGSSWTTYFDF (SEQ ID NO: 3) | QNNNGGSTFTGFP (SEQ ID NO: 6) |
| CDR1 | FDFSSNGMC (SEQ ID NO: 7) | QSINNELS (SEQ ID NO: 10) |
| CDR2 | WIACIYVDSSDNTNY (SEQ ID NO: 8) | LLIYGASTLES (SEQ ID NO: 11) |
| CDR3 | RGYGYVGSAMDL (SEQ ID NO: 9) | QSAYYSSSSSYANT (SEQ ID NO: 12) |
| CDR1 | FTISSIWIC (SEQ ID NO: 13) | ESISNLLA (SEQ ID NO: 16) |
| CDR2 | WIACIYAGSDGGSYY (SEQ ID NO: 14) | VLIYKASALPS (SEQ ID NO: 17) |
| CDR3 | RASNSWQYGYAGYGNYKDYFNL (SEQ | QSYYGSSDTGNT (SEQ ID NO: 18) |
| | ID NO: 15) | |
| CDR1 | FSFSSSYWIS (SEQ ID NO: 19) | QSISSYLA (SEQ ID NO: 22) |
| CDR2 | WIACIAIGSSGTTYY (SEQ ID NO: 20) | LLIYRASTLAS (SEQ ID NO: 23) |
| CDR3 | RSGDGYTYVEL (SEQ ID NO: 21) | QNYYDIDDSDNT (SEQ ID NO: 24) |
| CDR1 | FSFSWIC (SEQ ID NO: 25) | QSISSWLA (SEQ ID NO: 28) |
| CDR2 | WIACIYAGSSAKTYY (SEQ ID NO: 26) | LLIYGTSTLAS (SEQ ID NO: 29) |
| CDR3 | RASNYYRYGVAGYADYTGYFNL (SEQ | QNYGGSSSGDA (SEQ ID NO: 30) |
| | ID NO: 27) | |
| CDR1 | FSFSSNYWIC (SEQ ID NO: 31) | QNIYSNLA (SEQ ID NO: 34) |
| CDR2 | WIACIATGSSGSTYY (SEQ ID NO: 32) | LLIYGASNLES (SEQ ID NO: 35) |
| CDR3 | RGEYTYGYVEYAIVTQYYFDL (SEQ ID | QSADYIGSAYNA (SEQ ID NO: 36) |
| | NO: 33) | |
| CDR1 | FSFSSSYYMC (SEQ ID NO: 37) | QSISNYLA (SEQ ID NO: 40) |
| CDR2 | WIACIYTTYGGTWY (SEQ ID NO: 38) | LLIYRASTLES (SEQ ID NO: 41) |
| CDR3 | RSSISDVTYFNL (SEQ ID NO: 39) | QSYYDNNNYA (SEQ ID NO: 42) |
| CDR1 | FTLSTYWVC (SEQ ID NO: 43) | QSVYNNNYLA (SEQ ID NO: 46) |
| CDR2 | WIGCIDTVSSGDTYF (SEQ ID NO: 44) | LLIYWASKLAS (SEQ ID NO: 47) |
| CDR3 | RRTGSGWTL (SEQ ID NO: 45) | LGAYVSNGWYFA (SEQ ID NO: 48) |
| CDR1 | FSFSSYWTC (SEQ ID NO: 49) | ESVYNNNQLS (SEQ ID NO: 52) |
| CDR2 | WLGCTDGGSSGDTYY (SEQ ID NO: 50) | LLIYWASKLAS (SEQ ID NO: 53) |
| CDR3 | RNLITWDL (SEQ ID NO: 51) | AGYKSSITDGNA (SEQ ID NO: 54) |
| CDR1 | FDFSTNIMC (SEQ ID NO: 55) | QSIISALA (SEQ ID NO: 58) |
| CDR2 | WIACIYAGDGSTYY (SEQ ID NO: 56) | LLIYAASTLAS (SEQ ID NO: 59) |
| CDR3 | RASTYWNYGYAGYGYYPGYFNL (SEQ | QTYAYSTKSNYGSV (SEQ ID NO: 60) |
| | ID NO: 57) | |
| CDR1 | FSFSSGYDMC (SEQ ID NO: 61) | EDIYSGLA (SEQ ID NO: 64) |
| CDR2 | WIACIYTGDGSTYY (SEQ ID NO: 62) | LLIYKASNLAS (SEQ ID NO: 65) |
| CDR3 | REDVSSGDYTFNL (SEQ ID NO: 63) | QQGVTYSNVDNT (SEQ ID NO: 66) |
| CDR1 | FSFSSTYYMC (SEQ ID NO: 67) | QNIYSNLA (SEQ ID NO: 70) |
| CDR2 | WIACIYTGSTGSTYY (SEQ ID NO: 68) | LLIFGASNLES (SEQ ID NO: 71) |
| CDR3 | RGDYTYAYAGGAHVTNYYFDL (SEQ ID | QTADYSSSTDWGA (SEQ ID NO: 72) |
| | NO: 69) | |
| CDR1 | FDFSSNGMC (SEQ ID NO: 73) | QSISNELS (SEQ ID NO: 76) |
| CDR2 | WIACIYVDSSDSTYY (SEQ ID NO: 74) | LLIYGASTLES (SEQ ID NO: 77) |
| CDR3 | RGYGYVGSAMDL (SEQ ID NO: 75) | QSAYYSSSSSYANT (SEQ ID NO: 78) |
| CDR1 | FTLSTYWVC (SEQ ID NO: 79) | QSVYNNNYLA (SEQ ID NO: 82) |
| CDR2 | WIGCIDTVSSGDTYF (SEQ ID NO: 80) | LLIYWASKLAS (SEQ ID NO: 83) |
| CDR3 | RRTGSGWTL (SEQ ID NO: 81) | LGAYVSNGWYFA (SEQ ID NO: 84) |
| CDR1 | FSFSSSYYMC (SEQ ID NO: 85) | QSVFSNDYFS (SEQ ID NO: 88) |
| CDR2 | WIACIYPDDGNTYY (SEQ ID NO: 86) | LLIYDASRLAS (SEQ ID NO: 89) |
| CDR3 | RALAYYAYVDGGHSYAINDFDL (SEQ ID | QGTYYSSAWYNA (SEQ ID NO: 90) |
| | NO: 87) | |
| CDR1 | FDFSSNGMC (SEQ ID NO: 91) | QSISNELS (SEQ ID NO: 94) |
| CDR2 | WIACIYVDSSDNTNY (SEQ ID NO: 92) | LLIYGASTLES (SEQ ID NO: 95) |
| CDR3 | RGYGYVGSAMDL (SEQ ID NO: 93) | QSAYYSSSSSYANT (SEQ ID NO: 96) |
| CDR1 | IDFSSDYYMC (SEQ ID NO: 97) | QSIGSLLA (SEQ ID NO: 100) |
| CDR2 | WIACIYTGSSDDTYY (SEQ ID NO: 98) | LLIYWASTLAS (SEQ ID NO: 101) |
| CDR3 | RGGYGGKDL (SEQ ID NO: 99) | QCTYGSSGSSSYLNA (SEQ ID NO: |
| | | 102) |
| CDR1 | FDFSSNGMC (SEQ ID NO: 103) | QSISNELA (SEQ ID NO: 106) |
| CDR2 | WIACIYVDSSDSTYY (SEQ ID NO: 104) | LLIYGASTLES (SEQ ID NO: 107) |
| CDR3 | RGYGYVGSAMDL (SEQ ID NO: 105) | QSAYYSSSSSYANT (SEQ ID NO: 108) |
| CDR1 | FTLSTYWVC (SEQ ID NO: 109) | ESVYNNNYLS (SEQ ID NO: 112) |
| CDR2 | WIGCIDTVSSGDTYF (SEQ ID NO: 110) | LLIYQASTLAS (SEQ ID NO: 113) |
| CDR3 | RRTGSRWTL (SEQ ID NO: 111) | LGAFVSNGWYFA (SEQ ID NO: 114) |
| CDR1 | FSFSSGYNIC (SEQ ID NO: 115) | HSISKYFS (SEQ ID NO: 118) |
| CDR2 | LIACIYTSSSGSTYY (SEQ ID NO: 116) | LLIYEASTLAS (SEQ ID NO: 119) |
| CDR3 | RGEAYYAYGYVGYAYYHGAFDP (SEQ | QSYYYGTSSSYA (SEQ ID NO: 120) |
| | ID NO: 117) | |
| CDR1 | FSFSSSYYMC (SEQ ID NO: 121) | QSISSYLA (SEQ ID NO: 124) |
| CDR2 | WIACIYAGSSGGTYY (SEQ ID NO: 122) | LLIYRASTLAS (SEQ ID NO: 125) |
| CDR3 | RAFSYYYSDGYTGYAYGL (SEQ ID NO: | QGAYYSSSSSYG (SEQ ID NO: 126) |
| | 123) | |
| CDR1 | FSFSGYDM (SEQ ID NO: 127) | QGSSLA (SEQ ID NO: 130) |
| CDR2 | WIACIHSSSGTYY (SEQ ID NO: 128) | LLIYAASYLA (SEQ ID NO: 131) |
| CDR3 | RDFSYTDDYISYVYATD (SEQ ID NO: | QSTYYSSSTDIRA (SEQ ID NO: 132) |
| | 129) | |
| CDR1 | FSFSSYWIC (SEQ ID NO: 133) | QIYNNLA (SEQ ID NO: 136) |
| CDR2 | WIACIYAGSSGTYY (SEQ ID NO: 134) | LLIYGASNLE (SEQ ID NO: 137) |
| CDR3 | RAEYIDGYADYTYTTLYFDL (SEQ ID | QSADLTSSINV (SEQ ID NO: 138) |
| | NO: 135) | |
| CDR1 | FSFNSNYWMC (SEQ ID NO: 139) | ESVYNNNHLA (SEQ ID NO: 142) |
| CDR2 | WIGCILFGNTDTYY (SEQ ID NO: 140) | LLIYLASILDS (SEQ ID: NO: 143) |
| CDR3 | RSVSGVGSAWNL (SEQ ID NO: 141) | AGYKGITIDGSA (SEQ ID NO: 144) |
| CDR1 | FDFSSYYWIC (SEQ ID NO: 145) | QSVYNVNLLA (SEQ ID NO: 148) |
| CDR2 | WIACIYGGSSGSTYY (SEQ ID NO: 146) | LLIYETSKLES (SEQ ID NO: 149) |
| CDR3 | RSLYTWRYADYAASTLNL (SEQ ID NO: | AGGYSSSKDNS (SEQ ID NO: 150) |
| | 147) | |
| CDR1 | FSFSSSYFMC (SEQ ID NO: 151) | QSISSYLS (SEQ ID NO: 154) |
| CDR2 | WIACIYTGDGNNYY (SEQ ID NO: 152) | LLIYRASTLAS (SEQ ID NO: 155) |
| CDR3 | RGGSYYAYGYAGYDYYPDAFDY (SEQ | QSYYYSSSGSYG (SEQ ID NO: 156) |
| | ID NO: 153) | |
| CDR1 | FSFNSYYMC (SEQ ID NO: 157) | QNIYSNLA (SEQ ID NO: 160) |
| CDR2 | WIACISGGSSDNTYY (SEQ ID NO: 158) | LLIYGASNLES (SEQ ID NO: 161) |
| CDR3 | RDIPRSGYFGCDL (SEQ ID NO: 159) | QSTVYNSNYANT (SEQ ID NO: 162) |
| CDR1 | FSFSSSYWIY (SEQ ID NO: 163) | QSVYDNNWLA (SEQ ID NO: 166) |
| CDR2 | WIACIYTASRGSIYY (SEQ ID NO: 164) | LLIYAASTLSS (SEQ ID NO: 167) |
| CDR3 | RGPDYTYGYIGDALTRLDL (SEQ ID NO: | AGGYSSTSDIEDNT (SEQ ID NO: 168) |
| | 165) | |
| CDR1 | FSFSSSYWIC (SEQ ID NO: 169) | ESINSWLA (SEQ ID NO: 172) |
| CDR2 | WIACIYAGSSGDTYY (SEQ ID NO: 170) | LLIYSASTLAS (SEQ ID NO: 173) |
| CDR3 | RAEYIDGYADYTYTTLYYFDL (SEQ ID | QSYYSFSRFA (SEQ ID NO: 174) |
| | NO: 171) | |
| CDR1 | FSFSSGYWIC (SEQ ID NO: 175) | QSISNALA (SEQ ID NO: 178) |
| CDR2 | WIACIYTGVGATYY (SEQ ID NO: 176) | LLIYSASTLES (SEQ ID NO: 179) |
| CDR3 | RDFGGSSGFYFNL (SEQ ID NO: 177) | QNYYGSTSSSYGVA (SEQ ID NO: 180) |
| CDR1 | FSFSSSYYMC (SEQ ID NO: 181) | ESIYSNLA (SEQ ID NO: 184) |
| CDR2 | WIACIYAGSTFSTYY (SEQ ID NO: 182) | LLIYLASTLAS (SEQ ID NO: 185) |
| CDR3 | RSDSYYTYGYAGYAYAIFNL (SEQ ID | QSAYYSSSADIA (SEQ ID NO: 186) |
| | NO: 183) | |
| CDR1 | LDFSSSYWIC (SEQ ID NO: 187) | QNIYNNLA (SEQ ID NO: 190) |
| CDR2 | WIGCIKTATETTVY (SEQ ID NO: 188) | LLIYGASNLES (SEQ ID NO: 191) |
| CDR3 | KTYADNGGYINL (SEQ ID NO: 189) | QSADLTSSINV (SEQ ID NO: 192) |
| CDR1 | FSFSSSYWIC (SEQ ID NO: 193) | QSVYDNNWLA (SEQ ID NO: 196) |
| CDR2 | WIACIYTASRDSIYY (SEQ ID NO: 194) | LLIYEASKLAS (SEQ ID NO: 197) |
| CDR3 | RGPYYSYAYIGDALTRLDL (SEQ ID NO: | AGGYSSSSDIEDNT (SEQ ID NO: 198) |
| | 195) | |
| CDR1 | FSFNSNYYMC (SEQ ID NO: 199) | QSVYNNNNLA (SEQ ID NO: 202) |
| CDR2 | WIACIYTGIVVPTYY (SEQ ID NO: 200) | LLIYSASSLAS (SEQ ID NO: 203) |
| CDR3 | RDPYVGSSYIYNL (SEQ ID NO: 201) | AGYKTYSNNENA (SEQ ID NO: 204) |
| CDR1 | FSFSSSYYMC (SEQ ID NO: 205) | ENIYSNLAW (SEQ ID NO: 208) |
| CDR2 | WIACIYAGSSSSTYY (SEQ ID NO: 206) | LLIYGASNLES (SEQ ID NO: 209) |
| CDR3 | RAGYIDSYVDYTYAAWYYFDL (SEQ ID | QSADLSSSINV (SEQ ID NO: 210) |
| | NO: 207) | |
| CDR1 | FSFSSSYYMC (SEQ ID NO: 303) | ESIYNNNNLG (SEQ ID NO: 306) |
| CDR2 | WIGCIYTGNDDTWY (SEQ ID NO: 304) | LLIYWASTLAS (SEQ ID NO: 307) |
| CDR3 | RGLSPIDL (SEQ ID NO: 305) | AGYKSRTTDGSAF (SEQ ID NO: 308) |
| CDR1 | FSFSSGYDMC (SEQ ID NO: 309) | QSIGSSLA (SEQ ID NO: 312) |
| CDR2 | WIACIHSSSGTTYY (SEQ ID NO: 310) | LLIYAASYLAS (SEQ ID NO: 313) |
| CDR3 | RDFSYTDDYISYVYATDL (SEQ ID NO: | QSTYYSSSTDIRA (SEQ ID NO: 314) |
| | 311) | |

Table 2 shows the combinations of V_{H} and V_{L} sequences (CDR1, 2 and 3 underlined) in an antigen-binding molecule that are derived from the 36 antibody clones.

| Clone ID | V_{H} | V_{L} |
|---|---|---|
| AB1C4 | QSLEESGGDLVKPGPSLTLTCKASG | DIVMTQTPASVEAAVGGTVTIKCQAG |
| | FTISNNYWICWVRQAPGKGLE | QNIDNYLSWYQQKPGQPPK |
| | WIGCIATGDGSTYYASWAKGRFTI | LLIYRASTLASGVPSRFKGSGSG |
| | SKTSSTTVTLQMTSLTAADTATYFCA | TEFTLTISDLECADAATYYC |
| | RGAAGSSWTTYFDFWGPGTPVTVSS | QNNNGGSTFTGFPFGGGTEVVVK |
| | (SEQ ID NO: 211) | (SEQ ID NO: 212) |
| AB1C10 | QSLEESGGDLVKPGASLTLTCTASG | DIVMTQTPASVEAAVGGTVTIKCQAS |
| | FDFSSNGMCWVRQAPGKGLE | QSINNELSWYQQKPGQRPK |
| | WIACIYVDSSDNTNYASWVNGRFTI | LLIYGASTLESGVPSRFSGSGSG |
| | SRTSSTTVTLQMTSLTAADTATYFCA | TEFTLTISDLECADAATYYC |
| | RGYGYVGSAMDLWGQGTLVTVSS | QSAYYSSSSSYANTFGGGTEVVVK |
| | (SEQ ID NO: 213) | (SEQ ID NO: 214) |
| AB1C11 | QSLEESGGDLVKPGASLTLTCKASG | FELTQTPSSVEAVVGGTVTINCQAS |
| | FTISSIWICWVRQAPGKGLE | ESISNLLAWYQQKSGQPPK |
| | WIACIYAGSDGGSYYASWARGRFTI | VLIYKASALPSGVSSRFKGSGSG |
| | SKTSSTTVTLQMTSLTAADTATYFCA | TEFTLTISDLECADAATYYC |
| | RASNSWQYGYAGYGNYKDYFNLWGP | QSYYGSSDTGNTFGGGTEVVVK |
| | GTLVTVSS | (SEQ ID NO: 216) |
| | (SEQ ID NO: 215) | |
| AB1C12 | QSLEESGGDLVKPGASLTLTCTASG | DVVMTQTPASVSEPVGGTVTIKCQAS |
| | FSFSSSYWISWVRQAPGKGLE | QSISSYLAWYQQKPGQPPK |
| | WIACIAIGSSGTTYYASWAKGRFT | LLIYRASTLASGVPSRFKGSGSGTQF |
| | ISKTSSTTVTLQMTSLTAADTATYFCA | TLTISDLECADAATYYC |
| | RSGDGYTYVELWGPGTLVTVSS | QNYYDIDDSDNTFGGGTEVVVK |
| | (SEQ ID NO: 217) | (SEQ ID NO: 218) |
| AB1C13 | QEQLEESGGDLVKPEGSLTLTCTASG | FELTQTPASVEAAVGGTVTIKCQAS |
| | FSFSWICWVRQAPGKGLE | QSISSWLAWYHQKPGQRPK |
| | WIACIYAGSSAKTYYASWAKGRFTI | LLIYGTSTLASGVPSRFKGSGSG |
| | SKASSTTVTLQMTSLTAADTATYFCA | TEFTLTISDLECADAATYYC |
| | RASNYYRYGVAGYADYTGYFNLWGP | QNYGGSSSGDAFGGGTEVVVK |
| | GTLVTVSS | (SEQ ID NO: 220) |
| | (SEQ ID NO: 219) | |
| AB1C14 | QSLEESGGDLVKPGASLTLTCTASG | FEMTQTPSSVSAAVGGTVTINCQAS |
| | FSFSSNYWICWVRQAPGKGLE | QNIYSNLAWYQQKPGQRPK |
| | WIACIATGSSGSTYYASWAKGRFTI | LLIYGASNLESGVPSRFKGSGSG |
| | SKTSSTTVTLQMTSLTAADTATYFCA | TEYTLTISDLECDDAATYYC |
| | RGEYTYGYVEYAIVTQYYFDLWGP | QSADYIGSAYNAFGGGTEVVVK |
| | GTLVTVSS | (SEQ ID NO: 222) |
| | (SEQ ID NO: 221) | |
| AB1C15 | QSLEESGGDLVKPGASLTLTCTASG | DIVMTQTPASVEAAVGGTVTIKCQAS |
| | FSFSSSYYMCWVRQAPGKGLE | QSISNYLAWYQQKPGQPPE |
| | WIACIYTTYGGTWYASWAKGRFTI | LLIYRASTLESGVPSRFKGSGSG |
| | SKTSSTTVTLQMTSLTDADTATYFCA | TGFTLTISDLECADAATYYC |
| | RSSISDVTYFNLWGPGTLVTVSS | QSYYDNNNYAFGGGTEVVVK |
| | (SEQ ID NO: 223) | (SEQ ID NO: 224) |
| AB1C17 | QEQLKESGGDLVKPGASLTLTCTASG | LVMTQTPSPVSAAVGGTVTISCQSS |
| | FTLSTYWVCWVRQAPGKGLE | QSVYNNNYLAWFQQNPGQPPK |
| | WIGCIDTVSSGDTYFASWAKGRFTG | LLIYWASKLASGVPSRFKGSGSG |
| | SKTSSTTVTLQMTSLTAADTATYFCA | TQFTLTISDVQCDDAATYYC |
| | RRTGSGWTLWGPGTLVTVSS | LGAYVSNGWYFAFGGGTEVVVK |
| | (SEQ ID NO: 225) | (SEQ ID NO: 226) |
| AB1C18 | QSLEESGGDLVKPGASLTLTCTASG | IVMTQTPSSKSVPVGDTVTINCQAS |
| | FSFSSYWTCWVRQAPGKGLE | ESVYNNNQLSWFQQKPGQPPK |
| | WLGCTDGGSSGDTYYATWAKGRVAI | LLIYWASKLASGVPSRFKGSGSG |
| | SKTSSTTVTLQVTSLTAADTATYFCA | TQFTLTISDVVCDDAATYYC |
| | RNLITWDLWGPGTLVTVSS | AGYKSSITDGNAFGGGTEVVVK |
| | (SEQ ID NO: 227) | (SEQ ID NO: 228) |
| AB1C19 | QSLEESGGDLVQPEGSLTLTCKASG | DIVMTQTPASVEAAVGGTVTIKCQAS |
| | FDFSTNIMCWVRQAPGKGLE | QSIISALAWYQQKPGQPPK |
| | WIACIYAGDGSTYYASWVNGRFTI | LLIYAASTLASGVPSRFKGSGSG |
| | SKTSSTTVTLQMTSLTAADTATYFCA | TQFTLTISDLECADAATYYC |
| | RASTYWNYGYAGYGYYPGYFNLWGP | QTYAYSTKSNYGSVFGGGTEVVVK |
| | GTLVTVSS | (SEQ ID NO: 230) |
| | (SEQ ID NO: 229) | |
| AB1C21 | QQQLVESGGGLVKPGASLTLTCKASG | YDMTQTPASVEVTVGGTVTIKCQAS |
| | FSFSSGYDMCWVRQAPGKGLE | EDIYSGLAWYQQKPGQRPK |
| | WIACIYTGDGSTYYASWARGRFTI | LLIYKASNLASGVPSRFSGSG |
| | SKTSSTTVTLQMTSLTAADTATYFCA | SGTEFTLTISGVECADAATYYC |
| | REDVSSGDYTFNLWGPGTLVTVSS | QQGVTYSNVDNTFGGGTEVVVK |
| | (SEQ ID NO: 231) | (SEQ ID NO: 232) |
| AB1C23 | QSLEESGGDLVKPGASLTLTCTASG | LVMTQTPSSVSAAVGGTVTINCQAS |
| | FSFSSTYYMCWVRQAPGKGLE | QNIYSNLAWYQQKPGQRPK |
| | WIACIYTGSTGSTYYASWAKGRFTG | LLIFGASNLESGVPSRFKGSGSG |
| | SKTSSTTVTLQMTSLTAADTATYFCA | TEFTLTISDLECDDAATYYC |
| | RGDYTYAYAGGAHVTNYYFDLWGP | QTADYSSSTDWGAFGGGTEVVVK |
| | GTLVTVSS | (SEQ ID NO: 234) |
| | (SEQ ID NO: 233) | |
| AB1C25 | QSLEESGGDLVKPGASLTLTCTASG | DIVMTQTPASVEAAVGGTVTIKCQAS |
| | FDFSSNGMCWVRQAPGKGLE | QSISNELSWYQQKPGQRPK |
| | WIACIYVDSSDSTYYASWVNGRFTI | LLIYGASTLESGVPSRFSGSGSG |
| | SRTSSTTVTLQMTSLTAADTATYFCA | TEFTLTISDLECADAATYYC |
| | RGYGYVGSAMDLWGQGTLVTVSS | QSAYYSSSSSYANTFGGGTEVVAA |
| | (SEQ ID NO: 235) | (SEQ ID NO: 236) |
| AB1C28 | QEQLKESGGDLVKPGASLTLTCTASG | LVMTQTPSPVSAAVGGTVTISCQSS |
| | FTLSTYWVCWVRQAPGKGLE | QSVYNNNYLAWFQQNPGQPPK |
| | WIGCIDTVSSGDTYFASWAKGRFTG | LLIYWASKLASGVPSRFKGSGSG |
| | SKTSSTTVTLQMTSLTAADTATYFCA | TQFTLTISDVQCDDAATYYC |
| | RRTGSGWTLWGPGTLVTVSS | LGAYVSNGWYFAFGGGIEVVVK |
| | (SEQ ID NO: 237) | (SEQ ID NO: 238) |
| AB1C29 | QEHLEESGGGLVKPEGSLTLTCTASG | QVLTQTPSSVSAAVGGTVTINCQSS |
| | FSFSSSYYMCWVRQAPGKGLE | QSVFSNDYFSWYQQKPGQPPK |
| | WIACIYPDDGNTYYASWAKGRFTI | LLIYDASRLASGVPSRFKGSGSG |
| | SKTSSTTVTLQMTSLTAADTATYFCA | TQFTLTISGVQCDDAATYYC |
| | RALAYYAYVDGGHSYAINDFDLWGP | QGTYYSSAWYNAFGGGTEVVVK |
| | GTLVTVSS | (SEQ ID NO: 240) |
| | (SEQ ID NO: 239) | |
| AB1C31 | QSLEESGGDLVKPGASLTLTCTASG | DIVMTQTPASVEAAVGGTVTIKCQAS |
| | FDFSSNGMCWVRQAPGKGLE | QSISNELSWYQQKPGQRPK |
| | WIACIYVDSSDNTNYASWVNGRFTI | LLIYGASTLESGVPSRFSGSGSG |
| | SRTSSTTVDLKMTSLTAADTATYFCA | TEFTLTISDLECADAATYYC |
| | RGYGYVGSAMDLWGQGTLVTVSS | QSAYYSSSSSYANTFGGGTEVVVK |
| | (SEQ ID NO: 241) | (SEQ ID NO: 242) |
| AB1C32 | QEQLEESGGDLVKPGGTLTLTCKASG | VVMTQTPASVEAAVGGTVTIKCQAS |
| | IDFSSDYYMCWVRQAPGKGLE | QSIGSLLAWYQQKPGQPPN |
| | WIACIYTGSSDDTYYASWAKGRFTI | LLIYWASTLASGVPSRFKGSGSG |
| | SKTSSPTVALQMTSLTAADTATYFCA | TEFTLTISDLECDDAATYYC |
| | RGGYGGKDLWGPGTLVTVSS | QCTYGSSGSSSYLNAFGGGTEVVVK |
| | (SEQ ID NO: 243) | (SEQ ID NO: 244) |
| AB1C34 | QSLEESGGDLVKPGASLTLTCTASG | NIVMTQTPSPVSGAVGGTVTIKCQAS |
| | FDFSSNGMCWVRQAPGKGLE | QSISNELAWFQQKPGQRPK |
| | WIACIYVDSSDSTYYASWVNGRFTI | LLIYGASTLESGVPSRFSGSGSG |
| | SKTSSTTVTLQMTSLTAADTATYFCA | TEFTLTISDLECADAATYYC |
| | RGYGYVGSAMDLWGQGTLVTVSS | QSAYYSSSSSYANTFGGGTEVVVK |
| | (SEQ ID NO: 245) | (SEQ ID NO: 246) |
| AB1C35 | QEQLKESGGDLVKPGASLTLTCTASG | QVLTQTPSSVSAGVGGTVTINCQAS |
| | FTLSTYWVCWVRQAPGKGLE | ESVYNNNYLSWYQQKPGQPPK |
| | WIGCIDTVSSGDTYFASWAKGRFTG | LLIYQASTLASGVPSRFKGSGSG |
| | SKTSSTTVTLQMTSLTAADTATYFCA | TQFTLTISDVQCDDAATYYC |
| | RRTGSRWTLWGPGTLVTVSS | LGAFVSNGWYFAFGGGTEVVVK |
| | (SEQ ID NO: 247) | (SEQ ID NO: 248) |
| AB1C36 | QSLEESGGDLVKPGASLTLTCTASG | DIVMTQTPASVEAGVGGTVTIKCQAS |
| | FSFSSGYNICWVRQAPGKGLE | HSISKYFSWYQQKIGQPPK |
| | LIACIYTSSSGSTYYASWAKGRFTI | LLIYEASTLASGVPSRFKGSGSG |
| | SKTSSTTVTLQMTSLTVADTATYFCA | TQFTLTISDLECADAATYYC |
| | RGEAYYAYGYVGYAYYHGAFDPWGP | QSYYYGTSSSYAFGGGTEVVVK |
| | GTLVTVSS | (SEQ ID NO: 250) |
| | (SEQ ID NO: 249) | |
| AB1C39 | QSLEESGGDLVKPGASLTLTCTASG | DIVMTQTPASVEAAVGGTVTIKCQAS |
| | FSFSSSYYMCWVRQAPGKGLE | QSISSYLAWYQQKPGQPPK |
| | WIACIYAGSSGGTYYASWAKGRFTI | LLIYRASTLASGVPSRFKGSGSG |
| | SKTSSTTVTLQMTSLTAADTATYFCA | TQFTLTISDLECADAATYYC |
| | RAFSYYYSDGYTGYAYGLWGP | QGAYYSSSSSYGFGGGTEVVVK |
| | GTLVTVSS | (SEQ ID NO: 252) |
| | (SEQ ID NO: 251) | |
| AB1C45 | QERLEESGGGLVQPEGSLTLTCTASG | IVMTQTPSSKSVPVGDTVTINCQA |
| | FSFSSSYYMCWVRQAPGKGMEWIGCI | SESIYNNNNLGWYQQKPGQPPKLL |
| | YTGNDDTWYASWAKGRFTVSKTSSTT | IYWASTLASGVPSRFKGSGSGTQF |
| | VTLQMTSLTATDTATYFCARGLSPID | TLTISDVECDDAATYYCAGYKSRT |
| | LWGPGTLVTVSS | TDGSAFGGGTEVVVK |
| | (SEQ ID NO: 315) | (SEQ ID NO: 316) |
| AB2C53 | QEQLVESGGGLVQPEGSLTLTCTASG | AIEMTQTPASVSAAVGGTVTIKCQ |
| | FSFSGYDMCWVRQAPGKGLE | ASQGSSLAWYQQKPGQPPK |
| | WIACIHSSSGTYYANWAKGRFTI | LLIYAASYLASVPSRFKGSGSG |
| | SKTSSTTVTLQMTSLAADTATYFCA | TEYTLTISGVQCADAAYYC |
| | RDFSYTDDYISYVYATDWGPGTLVTVSS | QSTYYSSSTDIRAFGGGTEVVVK |
| | (SEQ ID NO: 253) | (SEQ ID NO: 254) |
| AB2C78 | QSLEESGGDLVKPGASLTLTCTASG | ALVMTQTPSSVSAAVGGTVTINCQ |
| | FSFSSYWICWVRQAPGKGLE | ASQIYNNLAWYQQKPGQRPK |
| | WIACIYAGSSGTYYASWAKGRFTI | LLIYGASNLEGVPSRFKGSGSG |
| | SKTSSTTVTLQTTSLAADTATYFCA | TEYTLTISDLECDDAAYYC |
| | RAEYIDGYADYTYTTLYFDLWGP | QSADLTSSINVFGGGTEVVVK |
| | GTPVTVSS | (SEQ ID NO: 256) |
| | (SEQ ID NO: 255) | |
| AB2C102 | QQQLEESGGDLVQPGASLTLTCTASG | IVMTQTPSSKSVPVGDTVTINCQ |
| | FSFNSNYWMCWGRQAPGKGLE | ASESVYNNNHLAWYQQKPGQSPK |
| | WIGCILFGNTDTYYANWAKGRFTI | LLIYLASILDSGVPSRFKGSGSG |
| | SKTSSTTVTLQMTSLTAADTATYFCA | TQFTLTISDVVCDDAATYYC |
| | RSVSGVGSAWNLWGPGTLVTVSS | AGYKGITIDGSAFGGGTELVVK |
| | (SEQ ID NO: 257) | (SEQ ID NO: 258) |
| AB2C103 | QSLEESGGDLVKPGASLTLTCTASG | AVLTQTPSPVSAAVGGTVSISCQ |
| | FDFSSYYWICWVRQAPGKGLE | SSQSVYNVNLLAWYQQKPGQPPK |
| | WIACIYGGSSGSTYYATWAKGRFTI | LLIYETSKLESGVPSRFSGSGSG |
| | SETSSTTVTLQMTSLTAADMATYFCA | TQFTLTISDVQCDDAATYYC |
| | RSLYTWRYADYAASTLNLWGPGTLVTVSS | AGGYSSSKDNSFGGGTEVVVK |
| | (SEQ ID NO: 259) | (SEQ ID NO: 260) |
| AB2C124 | QEQLVESGGGLVQPEGSLTLTCTASG | DIVMTQTPASVEVAVGGTVTIK |
| | FSFSSSYFMCWVRQAPGKGLE | CQASQSISSYLSWYQQKPGQPPK |
| | WIACIYTGDGNNYYASWAKGRFTI | LLIYRASTLASGVPSRFKGSGSG |
| | SKTSSTTVTLQMTSLTAADTATYFCS | TQFTLTISDLECADAATYYC |
| | RGGSYYAYGYAGYDYYPDAFDYWGP | QSYYYSSSGSYGFGGGTEVVVK |
| | GTLVTVSS | (SEQ ID NO: 262) |
| | (SEQ ID NO: 261) | |
| AB2C127 | QSLEESGGDLVKPGASLTLTCTASG | VVMTQTPASVSEPVGGTVTIKCQ |
| | FSFNSYYMCWVRQAPGKGLE | ASQNIYSNLAWYQQKPGQRPK |
| | WIACISGGSSDNTYYASWAKGRFTT | LLIYGASNLESGVPSRFKGSGSG |
| | SKTSSTTVTLQMTSLTAADTATYFCA | TEYTLTISNLECADAATYYC |
| | RDIPRSGYFGCDLWGPGTLVTVSS | QSTVYNSNYANTFGGGTEVVVK |
| | (SEQ ID NO: 263) | (SEQ ID NO: 264) |
| AB2C128 | QSLEESGGDLVKPGASLTLTCTASG | AVLTQTPSPVSAAVGGTVSISCQ |
| | FSFSSSYWIYWVRQAPGKGLE | SSQSVYDNNWLAWYQQKAGQPPK |
| | WIACIYTASRGSIYYASWTKGRFTI | LLIYAASTLSSGVPSRFKGSGSG |
| | SKTSSTTVTLQMTSLTAADTATYFCA | IEFTLTISDVQCDDAATYYC |
| | RGPDYTYGYIGDALTRLDLWGQGTLVTVSS | AGGYSSTSDIEDNTFGGGTEVVVK |
| | (SEQ ID NO: 265) | (SEQ ID NO: 266) |
| AB2C129 | QSLEESGGDLVKPGASLTLTCTASG | DVVMTQTPASVSEPVGGTVTINCQ |
| | FSFSSSYWICWVRQAPGKGLE | ASESINSWLAWYQQKPGQPPK |
| | WIACIYAGSSGDTYYASWAKGRFTI | LLIYSASTLASGVPSRFKGSGSG |
| | SKTSSTTVTLQTTSLTAADTATYFCA | IEFTLTISDLECADAATYFC |
| | RAEYIDGYADYTYTTLYYFDLWGP | QSYYSFSRFAFGGGTEVVVK |
| | GTPVTVSS | (SEQ ID NO: 268) |
| | (SEQ ID NO: 267) | |
| AB2C130 | QSLEESGGDLVKPGASLTLTCTASG | FELTQTPSSVEAAVGATVTIKCQAS |
| | FSFSSGYWICWVRQAPGKGLE | QSISNALAWYQQKPGQPPK |
| | WIACIYTGVGATYYASWAKGRFTI | LLIYSASTLESGVPSRFKGSGSG |
| | SKTSSTTVTLQMTSLTAADTATYFCA | TEFTLTISDLECADAATYYC |
| | RDFGGSSGFYFNLWGPGTLVTVSS | QNYYGSTSSSYGVAFGGGTEVVVK |
| | (SEQ ID NO: 269) | (SEQ ID NO: 270) |
| AB2C131 | QSLEESGGGLVQPEGSLTLTCTASG | LVMTQTPSSVSAAVGGTVTINCQAS |
| | FSFSSSYYMCWVRQAPGKGLE | ESIYSNLAWYQQKPGQPPK |
| | WIACIYAGSTFSTYYASWAKGRFTI | LLIYLASTLASGVPSRFKGSGSG |
| | SKTSSTTVTLQMTSLTAADTATYFCA | TEFTLTISDLECADAATYYC |
| | RSDSYYTYGYAGYAYAIFNLWGP | QSAYYSSSADIAFGGGTEVVVK |
| | GTLVTVSS | (SEQ ID NO: 272) |
| | (SEQ ID NO: 271) | |
| AB2C133 | QSLEESGGDLVKPGASLTLTCTASG | LVMTQTPSSVSAAVGGTVTINCQAS |
| | LDFSSSYWICWVRQAPGKGLE | QNIYNNLAWYQQKPGQRPK |
| | WIGCIKTATETTVYASWAKGRFTI | LLIYGASNLESGVPSRFKGSGSG |
| | SKTSSTTVTLQMTSLTAADTATYLCA | TEYTLTISDLECDDAATYYC |
| | KTYADNGGYINLWGPGTLVTVSS | QSADLTSSINVFGGGTEVVVK |
| | (SEQ ID NO: 273) | (SEQ ID NO: 274) |
| AB2C135 | QSLEESGGDLVKPGASLTLTCTASG | AVLTQTPSPVSAAVGGTVSISCQSS |
| | FSFSSSYWICWVRQAPGKGLE | QSVYDNNWLAWYQQKPGQPPK |
| | WIACIYTASRDSIYYASWAKGRFTI | LLIYEASKLASGVPSRFKGSGSG |
| | SKTSSTTVTLQMTSLTAADTATYFCA | TQFTLTISGVQCDDASTYYC |
| | RGPYYSYAYIGDALTRLDLWGQGTLVTVSS | AGGYSSSSDIEDNTFGGGTEVVVK |
| | (SEQ ID NO: 275) | (SEQ ID NO: 276) |
| AB2C136 | QEQLEESGGDLVKPGASLTLTCTASG | LVMTQTPSPVSAAVGSTVTISCQSS |
| | FSFNSNYYMCWVRQAPGKGLE | QSVYNNNNLAWYQQKPGQPPK |
| | WIACIYTGIVVPTYYASWAKGRFTI | LLIYSASSLASGVPSRFKGSGSG |
| | SKTSSTTVTLQMTSLTAADTATYFCA | TQFTLTISGVECDDAATYYC |
| | RDPYVGSSYIYNLWGPGTLVTVSS | AGYKTYSNNENAFGGGTEVVVK |
| | (SEQ ID NO: 277) | (SEQ ID NO: 278) |
| AB2C138 | QSLEESGGDLVKPGASLTLTCTASG | QVLTQTPSSVSEPVGGTVTINCQAS |
| | FSFSSSYYMCWVRQAPGKGLE | ENIYSNLAWYHQKPGQRPK |
| | WIACIYAGSSSSTYYASWAKGRFTI | LLIYGASNLESGVPSRFKGSGSG |
| | SKTSSTTVTLQTTSLTAADTATYFCA | TEYTLYHQTISDLECDDAATYYC |
| | RAGYIDSYVDYTYAAWYYFDLWGP | QSADLSSSINVFGGGTEVVVK |
| | GTLVTVSS | (SEQ ID NO: 280) |
| | (SEQ ID NO: 279) | |

Table 3 provides some examples of V_{H}, V_{L} sequences of humanized clones

| | V_{H} | V_{L} |
|---|---|---|
| hC4 | EVQLVESGGGLVQPGGSLRLSCAASG | DIQMTQSPSSLSASVGDRVTITC |
| | FTISNNYWICWVRQAPGKGLEWIG | QAGQNIDNYLSWYQQKPGKVPK |
| | CIATGDGSTYYASWAKGRFTISRDN | LLIYRASTLASGVPSRFSGSGSG |
| | SKNTLYLQMNSLRAEDTAVYYCA | TDFTLTISSLQPEDVATYYC |
| | RGAAGSSWTTYFDFWGQGTLVTVSS | QNNNGGSTFTGFPFGQGTKVEIK |
| | (SEQ ID NO: 281) | (SEQ ID NO: 282) |
| hC12 | EVQLVESGGGLVQPGGSLRLSCAASG | DIQMTQSPSSLSASVGDRVTITCQ |
| | FSFSSSYWISWVRQAPGKGLEWIAC | ASQSISSYLAWYQQKPGKVPK |
| | IAIGSSGTTYYASWAKGRFTISRDN | LLIYRASTLASGVPSRFSGSGSG |
| | SKNTLYLQMNSLRAEDTAVYYCA | TDFTLTISSLQPEDVATYYC |
| | RSGDGYTYVELWGQGTLVTVSS | QNYYDIDDSDNTFGQGTKVEIK |
| | (SEQ ID NO: 283) | (SEQ ID NO: 284) |
| hC15 | EVQLVESGGGLVQPGGSLRLSCAA | DIQMTQSPSSLSASVGDRVTITCQ |
| | SGFSFSSSYYMCWVRQAPGKGLEWI | ASQSISNYLAWYQQKPGKVPK |
| | ACIYTTYGGTWYASWAKGRFTISR | LLIYRASTLESGVPSRFSGSGSG |
| | DNSKNTLYLQMNSLRAEDTAVYYCA | TDFTLTISSLQPEDVATYYC |
| | RSSISDVTYFNLWGQGTLVTVSS | QSYYDNNNYAFGQGTKVEIK |
| | (SEQ ID NO: 285) | (SEQ ID NO: 286) |
| hC18 | EVQLVESGGGLVQPGGSLRLSCAA | DIQMTQSPSSLSASVGDRVTITCQA |
| | SGFSFSSYWTCWVRQAPGKGLEWL | SESVYNNNQLSWYQQKPGKVPK |
| | GCTDGGSSGDTYYATWAKGRFTIS | LLIYWASKLASGVPSRFSGSGSG |
| | RDNSKNTLYLQMNSLRAEDTAVYY | TDFTLTISSLQPEDVATYYC |
| | CARNLITWDLWGQGTLVTVSS | AGYKSSITDGNAFGQGTKVEIK |
| | (SEQ ID NO: 287) | (SEQ ID NO: 288) |
| hC31 | EVQLVESGGGLVQPGGSLRLSCAAS | DIQMTQSPSSLSASVGDRVTITCQ |
| | GFDFSSNGMCWVRQAPGKGLEWIAC | ASQSISNELSWYQQKPGKVPK |
| | IYVDSSDNTNYASWVNGRFTISRDN | LLIYGASTLESGVPSRFSGSGSG |
| | SKNTLYLQMNSLRAEDTAVYYCA | TDFTLTISSLQPEDVATYYC |
| | RGYGYVGSAMDLWGQGTLVTVSS | QSAYYSSSSSYANTFGQGTKVEIK |
| | (SEQ ID NO: 289) | (SEQ ID NO: 290) |
| hC36 | EVQLVESGGGLVQPGGSLRLSCAASG | DIQMTQSPSSLSASVGDRVTITC |
| | FSFSSGYNICWVRQAPGKGLELI | QASHSISKYFSWYQQKPGKVPK |
| | ACIYTSSSGSTYYASWAKGRFTISR | LLIYEASTLASGVPSRFSGSGSG |
| | DNSKNTLYLQMNSLRAEDTAVYYCA | TDFTLTISSLQPEDVATYYC |
| | RGEAYYAYGYVGYAYYHGAFDPWGQ | QSYYYGTSSSYAFGQGTKVEIK |
| | GTLVTVSS | (SEQ ID NO: 292) |
| | (SEQ ID NO: 291) | |
| hC53 | EVQLVESGGGLVQPGGSLRLSCAA | DIQMTQSPSSLSASVGDRVTITCQAS |
| | SGFSFSSGYDMCWVRQAPGKGLE | QSIGSSLAWYQQKPGKVPKLLIYAAS |
| | WIACIHSSSGTTYYASWAKGRFTIS | YLASGVPSRFSGSGSGTDFTLTISSL |
| | RDNSKNTLYLQMNSLRAEDTAVYY | QPEDVATYYCQSTYYSSSTDIRAFGQ |
| | CARDFSYTDDYISYVYATDLWGQ | GTKVEIK |
| | GTLVTVSS | (SEQ ID NO: 318) |
| | (SEQ ID NO: 317) | |
| hC131 | EVQLVESGGGLVQPGGSLRLSCAASG | DIQMTQSPSSLSASVGDRVTITCQ |
| | FSFSSSYYMCWVRQAPGKGLEWIAC | ASESIYSNLAWYQQKPGKVPK |
| | IYAGSTFSTYYASWAKGRFTISRDN | LLIYEASTLASGVPSRFSGSGSG |
| | SKNTLYLQMNSLRAEDTAVYYCA | TDFTLTISSLQPEDVATYYC |
| | RSDSYYTYGYAGYAYAIFNLWG | QSAYYSSSADIAFGQGTKVEIK |
| | QGTLVTVSS | |
| | (SEQ ID NO: 293) | (SEQ ID NO: 294) |

The antigen-binding molecules of the present invention may be in isolated, purified, synthetic or recombinant form. Suitable antigen-binding molecules may be selected from antibodies and their antigen-binding fragments, including monoclonal antibodies (MAbs), chimeric antibodies, humanized antibodies, human antibodies, and antigen-binding fragments of such antibodies. The antigen-binding molecules may be multivalent (e.g., bivalent) or monovalent. In some embodiments, the antigen-binding molecules comprise an Fc domain. In other embodiments, the antigen-binding molecules lack an Fc domain. In some embodiments, the antigen-binding molecules are monovalent antigen-binding molecules (e.g., Fab, scFab, Fab′, scFv, one-armed antibodies, etc.).

By “antigen-binding molecule” is meant a molecule that has binding affinity for a target antigen. It will be understood that this term extends to immunoglobulins, immunoglobulin fragments and non-immunoglobulin derived protein frameworks that exhibit antigen-binding activity. Representative antigen-binding molecules that are useful in the practice of the present invention include antibodies and their antigen-binding fragments. The term “antigen-binding molecule” includes antibodies and antigen-binding fragments of antibodies.

The antigen-binding molecules as defined herein can be naked or conjugated to other molecules or moieties such as toxins, radioisotopes, small molecule drugs, polypeptides, etc.

The term “antibody”, as used herein, means any antigen-binding molecule or molecular complex comprising at least one complementarity determining region (CDR) that binds specifically to or interacts with a particular antigen. The term “antibody” includes full-length immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (which may be abbreviated as HCVR or V_{H}) and a heavy chain constant region. The heavy chain constant region comprises three domains, C_{H}1, C_{H}2 and C_{H}3. Each light chain comprises a light chain variable region (which may be abbreviated as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the invention, the FRs of an antibody of the invention (or antigen-binding portion thereof) may be identical to the human germline sequences, or may be naturally or artificially modified. An amino acid consensus sequence may be defined based on a side-by-side analysis of two or more CDRs.

An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called α, δ, ε, γ, and μ, respectively. The subunit structures and three-dimensional configurations of different classes of immunoglobulins are well known.

As used herein, the term “complementarity determining regions” (CDRs; i.e., CDR1, CDR2, and CDR3) refers to the amino acid residues of an antibody variable domain the presence of which are necessary for antigen-binding. Each variable domain typically has three CDR regions identified as CDR1, CDR2 and CDR3. Each complementarity determining region may comprise amino acid residues from a “complementarity determining region” as defined for example by Kabat (i.e., about residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e., about residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). In some instances, a complementarity determining region can include amino acids from both a CDR region defined according to Kabat and a hypervariable loop.

A “humanized” antibody refers to an antibody comprising amino acid residues from non-human CDRs and amino acid residues from human FRs. In certain embodiments, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally may comprise at least a portion of an antibody constant region derived from a human antibody.

As used herein, a “chimeric” molecule is one which comprises one or more unrelated types of components or contain two or more chemically distinct regions which can be conjugated to each other, fused, linked, translated, attached via a linker, chemically synthesized, expressed from a nucleic acid sequence, etc. For example, a peptide and a nucleic acid sequence, a peptide and a detectable label, unrelated peptide sequences, and the like. In embodiments in which the chimeric molecule comprises amino acid sequences of different origin, the chimeric molecule includes (1) polypeptide sequences that are not found together in nature (i.e., at least one of the amino acid sequences is heterologous with respect to at least one of its other amino acid sequences), or (2) amino acid sequences that are not naturally adjoined. For example, a “chimeric” antibody” as used herein refers to an antibody in which a portion of the heavy and/or light chain is derived from a particular source or species, while the remainder of the heavy and/or light chain is derived from a different source or species.

As used herein, the term “antigen” and its grammatically equivalents expressions (e.g., “antigenic”) refer to a compound, composition, or substance that may be specifically bound by the products of specific humoral or cellular immunity, such as an antibody molecule or T-cell receptor. Antigens can be any type of molecule including, for example, haptens, simple intermediary metabolites, sugars (e.g., oligosaccharides), lipids, and hormones as well as macromolecules such as complex carbohydrates (e.g., polysaccharides), phospholipids, and proteins. Common categories of antigens include, but are not limited to, viral antigens, bacterial antigens, fungal antigens, protozoa and other parasitic antigens, tumor antigens, antigens involved in autoimmune disease, allergy and graft rejection, toxins, and other miscellaneous antigens.

An “antigen-binding site” refers to the site, i.e., one or more amino acid residues, of an antigenbinding molecule which provides interaction with the antigen. For example, the antigen binding site of an antibody comprises amino acid residues from the complementarity determining regions (CDRs). A native immunoglobulin molecule typically has two antigen binding sites, a Fab molecule typically has a single antigen binding site. An antigen-binding site of an antigen-binding molecule described herein typically binds specifically to an antigen and more particularly to an epitope of the antigen.

The terms “antigen-binding fragment”, “antigen-binding portion”, “antigen-binding domain” and “antigen-binding site” are used interchangeably herein to refer to a part of an antigen-binding molecule that participates in antigen-binding. These terms include any naturally occurring, enzymatically obtainable, synthetic, or genetically engineered polypeptide or glycoprotein that specifically binds an antigen to form a complex.

Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, one-armed antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3, (vi) V_{H}-C_{H}2-C_{H}3; (vii) V_{H}-C_{L}; (viii) V_{L}-C_{H}1; (ix) V_{L}-C_{H}2, (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)). A multispecific antigen-binding molecule will typically comprise at least two different variable domains, wherein each variable domain is capable of specifically binding to a separate antigen or to a different epitope on the same antigen. Any multispecific antigen-binding molecule format, including bispecific antigen-binding molecule formats, may be adapted for use in the context of an antigen-binding fragment of an antibody of the present disclosure using routine techniques available in the art.

The term “variable region” or “variable domain” refers to the domain of an antibody heavy or light chain that is involved in binding the antigen-binding molecule to antigen. The variable domains of the heavy chain and light chain (V_{H} and V_{L}, respectively) of a native antibody generally have similar structures, with each domain comprising four conserved framework regions (FRs) and three hypervariable regions (HVRs). See, e.g., Kindt et al., Kuby Immunology, 6th ed., W.H. Freeman and Co., page 91 (2007). A single V_{H} or V_{L} domain may be sufficient to confer antigen-binding specificity.

The term “constant domains” or “constant region” as used herein denotes the sum of the domains of an antibody other than the variable region. The constant region is not directly involved in binding of an antigen, but exhibits various immune effector functions.

The term “bispecific antigen-binding molecule” refers to a multi-specific antigen-binding molecule having the capacity to bind to two distinct epitopes on the same antigen or on two different antigens. A bispecific antigen-binding molecule may be bivalent, trivalent, or tetravalent. As used herein, “valent”, “valence”, “valencies”, or other grammatical variations thereof, mean the number of antigen-binding sites in an antigen-binding molecule. These antigen recognition sites may recognize the same epitope or different epitopes. Bivalent and bispecific molecules are described in, e.g., Kostelny et al., 1992. J Immunol 148:1547; Pack and Pluckthun, 1992. Biochemistry 31:1579, Gruber et al. 1994. J Immunol 5368, Zhu et al. 1997. Protein Sci 6:781, Hu et al., 1996. Cancer Res. 56:3055, Adams et al., 1993. Cancer Res. 53:4026, and McCartney et al., 1995. Protein Eng. 8:301. Trivalent bispecific antigen-binding molecules and tetravalent bispecific antigen-binding molecules are also known in the art. See, e.g., Kontermann RE (ed.), Springer Heidelberg Dordrecht London New York, pp. 199-216 (2011). A bispecific antigen-binding molecule may also have valencies higher than 4 and are also within the scope of the present invention. Such antigen-binding molecules may be generated by, for example, dock and lock conjugation method. (Chang, C.-H. et al. In: Bispecific Antibodies. Kontermann RE (2011), supra).

The phrase “specifically binds” or “specific binding” refers to a binding reaction between two molecules that is at least two times the background and more typically more than 10 to 100 times background molecular associations under physiological conditions. When using one or more detectable binding agents that are proteins, specific binding is determinative of the presence of the protein, in a heterogeneous population of proteins and other biologics. Thus, under designated immunoassay conditions, the specified antigen-binding molecule binds to a particular antigenic determinant, thereby identifying its presence. Specific binding to an antigenic determinant under such conditions requires an antigen-binding molecule that is selected for its specificity to that determinant. This selection may be achieved by subtracting out antigen-binding molecules that cross-react with other molecules. A variety of immunoassay formats may be used to select antigen-binding molecules (e.g., immunoglobulins) [such that they are specifically immunoreactive with a particular antigen. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity). Methods of determining binding affinity and specificity are also well known in the art (see, for example, Harlow and Lane, supra); Friefelder, “Physical Biochemistry: Applications to biochemistry and molecular biology” (W.H. Freeman and Co. 1976)).

In one embodiment, the antigen-binding molecule specifically binds to a cell expressing ALPPL2 with an affinity of between about 14 pm to about 10 nM.

“Affinity” or “binding affinity” refers to the strength of the sum total of non-covalent interactions between a single binding site of a molecule (e.g., an antigen-binding molecule) and its binding partner (e.g., an antigen). Unless indicated otherwise, as used herein, “binding affinity” refers to intrinsic binding affinity which reflects a 1:1 interaction between members of a binding pair e.g., an antigen-binding molecule). The affinity of a molecule X for its partner Y can generally be represented by the dissociation constant (Kd), which is the ratio of dissociation and association rate constants (k_{off} and kₒₙ, respectively). Thus, equivalent affinities may comprise different rate constants, as long as the ratio of the rate constants remains the same. Affinity can be measured by common methods known in the art, including those described herein. A particular method for measuring affinity is Surface Plasmon Resonance (SPR).

The terms “polypeptide”, “peptide”, or “protein” are used interchangeably herein to designate a linear series of amino acid residues connected one to the other by peptide bonds between the alpha-amino and carboxy groups of adjacent residues. The amino acid residues are usually in the natural “L” isomeric form. However, residues in the “D” isomeric form can be substituted for any L-amino acid residue, as long as the desired functional property is retained by the polypeptide.

As used herein, the term “modified antibody” includes synthetic forms of antibodies which are altered such that they are not naturally occurring, e.g., antibodies that comprise at least two heavy chain portions but not two complete heavy chains (such as, domain deleted antibodies or minibodies); multispecific forms of antibodies (e.g., bispecific, trispecific, etc.) altered to bind to two or more different antigens or to different epitopes on a single antigen); heavy chain molecules joined to scFv molecules and the like. ScFv molecules are known in the art and are described, e.g., in U.S. Pat. No. 5,892,019. In addition, the term “modified antibody” includes multivalent forms of antibodies (e.g., trivalent, tetravalent, etc., antibodies that bind to three or more copies of the same antigen).

In one embodiment, the antigen-binding molecule specifically binds to rhesus macaque ALPPL2. The rhesus macaque ALPPL2 may have a sequence as shown in Genbank ID XP_011726419.1.

In one embodiment, the antigen-binding molecule comprises: (a) a V_{H} amino acid sequence having at least 90% (including at least 91% to 100% and all integer percentages therebetween) sequence identity to a V_{H} amino acid sequence as shown in any of the rows in Table 2 or Table 3, and (b) a V_{L} amino acid sequence having at least 90% sequence identity (including at least 91% to 100% and all integer percentages therebetween) to a V_{L} amino acid sequence as shown in the same row as the V_{H} amino acid sequence in Table 2 or Table 3.

In one embodiment, the antigen-binding molecule specifically binds ALPPL2 and ALPP but not ALPL or ALPI.

The antigen-binding molecule may, for example, comprise:
a) a V_{H} amino acid sequence having at least 90% sequence identity to SEQ ID NO: 281 and a V_{L} amino acid sequence having at least 90% sequence identity to SEQ ID NO: 282,
b) a V_{H} amino acid sequence having at least 90% sequence identity to SEQ ID NO: 283 and a V_{L} amino acid sequence having at least 90% sequence identity to SEQ ID NO: 284,
c) a V_{H} amino acid sequence having at least 90% sequence identity to SEQ ID NO: 285 and a V_{L} amino acid sequence having at least 90% sequence identity to SEQ ID NO: 286,
d) a V_{H} amino acid sequence having at least 90% sequence identity to SEQ ID NO: 287 and a V_{L} amino acid sequence having at least 90% sequence identity to SEQ ID NO: 288,
e) a V_{H} amino acid sequence having at least 90% sequence identity to SEQ ID NO: 289 and a V_{L} amino acid sequence having at least 90% sequence identity to SEQ ID NO: 290,
f) a V_{H} amino acid sequence having at least 90% sequence identity to SEQ ID NO: 291 and a V_{L} amino acid sequence having at least 90% sequence identity to SEQ ID NO: 292, or
g) a V_{H} amino acid sequence having at least 90% sequence identity to SEQ ID NO: 293 and a V_{L} amino acid sequence having at least 90% sequence identity to SEQ ID NO: 294.

The phrase “at least 90% sequence identity” as referred to in the specification may include at least 91% to 100% and all integer percentages therebetween.

In one embodiment, the antigen-binding molecule does not bind to ALPP. The antigen-binding molecule may bind to ALPPL2 but not ALPP. In one embodiment, the antigen-binding molecule binds to ALPPL2 but not ALPP, ALPL or ALPI. The antigen-binding molecule may comprise:
a) a heavy chain variable region (V_{H}) comprising the amino acid sequences of SEQ ID NO: 31, SEQ ID NO: 32 and SEQ ID NO: 33, and a light chain variable region (V_{L}) comprising the amino acid sequences of SEQ ID NO: 34, SEQ ID NO: 35 and SEQ ID NO: 36,
b) a heavy chain variable region (V_{H}) comprising the amino acid sequences of SEQ ID NO: 67, SEQ ID NO: 68 and SEQ ID NO: 69, and a light chain variable region (V_{L}) comprising the amino acid sequences of SEQ ID NO: 70, SEQ ID NO: 71 and SEQ ID NO: 72,
c) a heavy chain variable region (V_{H}) comprising the amino acid sequences of SEQ ID NO: 85, SEQ ID NO: 86 and SEQ ID NO: 87, and a light chain variable region (V_{L}) comprising the amino acid sequences of SEQ ID NO: 88, SEQ ID NO: 89 and SEQ ID NO: 90,
d) a heavy chain variable region (V_{H}) comprising the amino acid sequences of SEQ ID NO: 127, SEQ ID NO: 128 and SEQ ID NO: 129, and a light chain variable region (V_{L}) comprising the amino acid sequences of SEQ ID NO: 130, SEQ ID NO: 131 and SEQ ID NO: 132,
e) a heavy chain variable region (V_{H}) comprising the amino acid sequences of SEQ ID NO: 133, SEQ ID NO: 134 and SEQ ID NO: 135, and a light chain variable region (V_{L}) comprising the amino acid sequences of SEQ ID NO: 136, SEQ ID NO: 137 and SEQ ID NO: 138,
f) a heavy chain variable region (V_{H}) comprising the amino acid sequences of SEQ ID NO: 169, SEQ ID NO: 170 and SEQ ID NO: 171, and a light chain variable region (V_{L}) comprising the amino acid sequences of SEQ ID NO: 172, SEQ ID NO: 173 and SEQ ID NO: 174; or
g) a heavy chain variable region (V_{H}) comprising the amino acid sequences of SEQ ID NO: 205, SEQ ID NO: 206 and SEQ ID NO: 207, and a light chain variable region (V_{L}) comprising the amino acid sequences of SEQ ID NO: 208, SEQ ID NO: 209 and SEQ ID NO: 210.

In one embodiment, the antibody comprises:
a) a V_{H} amino acid sequence having at least 90% sequence identity to SEQ ID NO: 221 and a V_{L} amino acid sequence having at least 90% sequence identity to SEQ ID NO: 222,
b) a V_{H} amino acid sequence having at least 90% sequence identity to SEQ ID NO: 223 and a V_{L} amino acid sequence having at least 90% sequence identity to SEQ ID NO: 224,
c) a V_{H} amino acid sequence having at least 90% sequence identity to SEQ ID NO: 239 and a V_{L} amino acid sequence having at least 90% sequence identity to SEQ ID NO: 240,
d) a V_{H} amino acid sequence having at least 90% sequence identity to SEQ ID NO: 253 and a V_{L} amino acid sequence having at least 90% sequence identity to SEQ ID NO: 254,
e) a V_{H} amino acid sequence having at least 90% sequence identity to SEQ ID NO: 255 and a V_{L} amino acid sequence having at least 90% sequence identity to SEQ ID NO: 256,
f) a V_{H} amino acid sequence having at least 90% sequence identity to SEQ ID NO: 267 and a V_{L} amino acid sequence having at least 90% sequence identity to SEQ ID NO: 268, or
g) a V_{H} amino acid sequence having at least 90% sequence identity to SEQ ID NO: 279 and a V_{L} amino acid sequence having at least 90% sequence identity to SEQ ID NO: 280.

In one embodiment, the antigen-binding molecule is an antibody or antigen-binding fragment thereof or a chimeric antigen receptor (CAR).

In one embodiment, the antibody or antigen-binding fragment thereof is humanized or chimerized.

In one embodiment, the antibody or antigen-binding fragment thereof is a humanized antibody comprising:

a) a heavy chain variable region that comprises:
i) a V_{H}FR1 having at least 90% sequence identity to

(SEQ ID NO: 295)
EVQLVESGGGLVQPGGSLRLSCAASG,

ii) a V_{H}FR2 having at least 90% sequence identity to WVRQAPGKGLE (SEQ ID NO: 296),
iii) a V_{H}FR3 having at least 90% sequence identity to

(SEQ ID NO: 297)
ASWAKGRFTISRDNSKNTLYLQMNSLRAEDTAVYYCA,

iv) a V_{H}FR4 having at least 90% sequence identity to WGQGTLVTVSS (SEQ ID NO: 298), and

b) a light chain variable region that comprises:
i) a V_{L}FR1 having at least 90% sequence identity to

(SEQ ID NO: 299)
DIQMTQSPSSLSASVGDRVTITCQAG

ii) a V_{H}FR2 having at least 90% sequence identity to WYQQKPGKVPK (SEQ ID NO: 300),
iii) a V_{H}FR3 having at least 90% sequence identity to

(SEQ ID NO: 301)
GVPSRFSGSGSGTDFTLTISSLQPEDVATYYC

i) a V_{H}FR4 having at least 90% sequence identity to FGQGTKVEIK (SEQ ID NO: 302).

In one embodiment, the antibody or antigen-binding fragment thereof comprises a C_{H}1 amino acid sequence having at least 90% (including at least 91% to 100% and all integer percentages therebetween) to:

(SEQ ID NO: 319)
ASTKGPSVFPLAPSSKSTSGGTAALGCLVKDYFPEPVTVSW
NSGALTSGVHTFPAVLQSSGLYSLSSVVTVPSSSLGTQTYI
CNVNHKPSNTKVDKK.

In one embodiment, the antibody or antigen-binding fragment thereof comprises a C_{L} amino acid sequence having at least 90% (including at least 91% to 100% and all integer percentages therebetween) to:

(SEQ ID NO: 320)
RTVAAPSVFIFPPSDEQLKSGTASVVCLLNNFYPREAKVQ
WKVDNALQSGNSQESVTEQDSKDSTYSLSSTLTLSKADYE
KHKVYACEVTHQGLSSPVTKSFNRGEC.

Representative antigen-binding molecules contemplated by the present disclosure include full-length immunoglobulins and antigen-binding fragments, including recombinant antigen-binding molecules, which may be monovalent or multivalent, monospecific or multispecific.

In one embodiment, the antibody or antigen-binding fragment thereof is a full-length antibody, a substantially intact antibody, a Fab fragment, scFab, Fab′, a single chain variable fragment (scFv) or a one-armed antibody.

In one embodiment, the antibody has an isotype selected from the group consisting of IgG1, IgG2, IgG3, and IgG4. In one embodiment, the antibody is an IgG1 antibody. The antibody may have antibody-dependent cell-mediated cytotoxicity (ADCC) activity and can induce NK cell killing. The heavy chain constant region can be a wild-type human Fc region, or a human Fc region that includes one or more amino acid substitutions. The antibodies can have mutations that stabilize the disulfide bond between the two heavy chains of an immunoglobulin, such as mutations in the hinge region of IgG4, as disclosed in the art (e.g., Angal et al., 1993. Mol. Immunol., 30:105-08). See also, e.g., U.S. 2005/0037000. The heavy chain constant region can also have substitutions that modify the properties of the antigen-binding molecule (e.g., decrease one or more of: Fc receptor binding, antigen-binding molecule glycosylation, deamidation, binding to complement, or methionine oxidation). In some instances, the antigen-binding molecules may have mutations such as those described in U.S. Pat. Nos. 5,624,821 and 5,648,260. In some embodiments, the antigen-binding molecule is modified to reduce or eliminate effector function.

In one embodiment, the antigen-binding molecule of the present invention is a monovalent antigen-binding molecule. Non-limiting monovalent antigen-binding molecules include: a Fab fragment consisting of V_{L}, V_{H}, C_{L} and C_{H}1 domains; a Fab′ fragment consisting of V_{L}, V_{H}, C_{L} and C_{H}1 domains, as well as a portion of a C_{H}2 domain; an Fd fragment consisting of V_{H} and C_{H}1 domains; an Fv fragment consisting of V_{L} and V_{H} domains of a single arm of an antibody; a single-chain antibody molecule (e.g., scFab and scFv); a single domain antibody (dAb) fragment (Ward et al., 1989 Nature 341:544-546), which consists of a V_{H} domain; and a one-armed antibody, such as described in US20080063641 (Genentech) or other monovalent antibody, e.g., such as described in WO2007048037 (Amgen).

In one embodiment, a monovalent antigen-binding molecule comprises an Fv fragment. The Fv fragment is the smallest unit of an immunoglobulin molecule with function in antigen-binding activities. An antigen-binding molecule in scFv (single chain fragment variable) format consists of variable regions of heavy (V_{H}) and light (V_{L}) chains, which are joined together by a flexible peptide linker that can be easily expressed in functional form in an expression host such as E. coli and mammalian cells, allowing protein engineering to improve the properties of scFv such as increase of affinity and alteration of specificity (Ahmed et al., 2012. Clin Dev Immunol. 2012:980250). Representative examples of linker sequences are described in Section 4.5 infra. In the scFv construction, the order of the domains can be either V_{H}-linker-V_{L} or V_{L}-linker-V_{H} and both orientations can applied.

In some embodiments, the linker sequences used in scFvs are multimers of the pentapeptide GGGGS [SEQ ID NO:66] (or G4S or Gly4Ser). Those include the 15-mer (G4S)3 (Huston et al., 1988. Proc Natl Acad Sci USA. 85(16), 5879-83), the 18-mer GGSSRSSSSGGGGSGGGG [SEQ ID NO:67] (Andris-Widhopf et al., “Generation of human scFv antibody libraries: PCR amplification and assembly of light- and heavy-chain coding sequences.” Cold Spring Harbor Protocols, 2011(9)) and the 20-mer (G4S)4 (Schaefer et al., “Construction of scFv Fragments from Hybridoma or Spleen Cells by PCR Assembly.” In: Antibody Engineering, R. Kontermann and S. Dubel, Springer Verlag, Heidelberg, Germany (2010) pp. 21-44). Many other sequences have been proposed, including sequences with added functionalities, e.g., an epitope tag or an encoding sequence containing a Cre-Lox recombination site or sequences improving scFv properties, often in the context of particular antibody sequences.

Cloning of the scFv is usually done by a two-step overlapping PCR (also known as Splicing by Overlap Extension or SOE-PCR), as described (Schaefer et al., 2010, supra). The V_{H} and V_{L} domains are first amplified and gel-purified and secondarily assembled in a single step of assembly PCR. The linker is generated either by overlap of the two inner primers or by adding a linker primer whose sequence covers the entire linker or more (three-fragment assembly PCR).

Single chain Fv (scFv) antigen-binding molecules may be recombinantly produced for example in E. coli, insect cells or mammalian host cells upon cloning of the protein coding sequence for the scFv in the context of appropriate expression vectors with appropriate translational, transcriptional start sites and, in the case of mammalian expression, a signal peptide sequence.

In one embodiment, the monovalent antigen-binding molecule comprises an Fab fragment. In an illustrative example of this type, the monovalent antigen-binding molecule is a one-armed antibody consisting or consisting essentially of a single antigen-binding fragment (Fab) and a Fc region, wherein the Fc region comprises a first and a second Fc polypeptide, and wherein the first and second Fc polypeptides are present in a complex.

Recombinant expression of Fc-containing monovalent antigen-binding molecules can often lead to undesirable bivalent, homodimer contaminants. Strategies to inhibit formation of homodimers are known including methods that introduce mutations into immunoglobulin constant regions to create altered structures that support unfavorable interactions between polypeptide chains and suppress unwanted Fc homodimer formation. Non-limiting examples of this strategy to promote heterodimerization include the introduction of knobs-into-holes (KIH) structures into the two polypeptides and utilization of the naturally occurring heterodimerization of the C_{L} and C_{H}1 domains (see, Kontermann, supra, pp. 1-28 (2011) Ridgway et al., 1996. Protein Eng. 9(7):617-21; Atwell et al., 1997. J Mol Biol. 270(1):26-35; as described in WO 2005/063816). These KIH mutations promote heterodimerization of the knob containing Fc and the hole containing heavy chain, improving the assembly of monovalent antibody and reducing the level of undesired bivalent antibody.

Modifications in the Fc domain of an antigen-binding molecules may also be desirable to reduce Fc receptor binding and therefore reduce the potential for FcγRIIa-mediated activation of platelets. For example, the so-called ‘LALA’ double mutation (Leu234Ala together with Leu235Ala) in human IgG (including IgG1) is known to significantly impair Fc receptor binding and effector function (Lund et al., 1991, J. Immunol. 147, 2657-2662; Lund et al., 1992, Mol. Immunol. 29:53-59). For human IgG4, engineering mutations S228P/L235E variant (SPLE) has previously demonstrated minimal FcγR binding (Newman et al., 2001, Clin. Immunol. 98, 164-174). Mutations in IgG1 or IgG4 Fc domains can be combined, for instance combining the LALA mutations in human IgG1 with a mutation at P329G or combining the SPLE mutation in human IgG4 with a mutation at P329G, completely abolished FcγR and C1q interactions (Schlothauer et al., 2016, Protein Eng Des. Sel. 29, 457-466).

In one embodiment, the antigen-binding molecule (e.g., a MAb or an antigen-binding fragment thereof), in which each of the IgG1 Fc chains of the antibody carries P329G, L235A, L234A (P329G LALA) mutations or each of the IgG4 Fc chains carries P329G, S228P, L235E mutations, in order to reduce or abolish any undesired cross-linking, platelet activation, or immune effector function (e.g., antibody-dependent cell-meditated cytotoxicity (ADCC), phagocytosis (ADCP) and complement dependent cytotoxicity (CDC)) of the antigen-binding molecule.

In one embodiment, each of the IgG1 Fc chains of the antigen-binding molecule (or antibody) carries mutations comprising a) S239D, A330L and 1332E or b) F243L, R292P, Y300L, V305I and P396L, which enhance immune effector function of the antigen-binding molecule (e.g. ADCC).

In one embodiment, the antigen-binding molecule (or antibody) comprises a CH₂—CH₃ sequence of having at least 70% sequence identity to an amino acid sequence of SEQ ID NO: 321, SEQ ID NO: 322 or SEQ ID NO: 323.

The antigen-binding molecules may comprise a heavy chain sequence. The heavy chain sequence may, for example, comprise or consist of a V_{H} sequence listed in Table 3 that is fused to a C_{H}1 sequence (e.g. SEQ ID NO: 319) and a C_{H}2-C_{H}3 sequence (e.g. SEQ ID NO: 321, SEQ ID NO: 322 or SEQ ID NO: 33). For example, the heavy chain sequence may comprise an amino acid sequence having at least 70% sequence identity to SEQ ID NO: 326, SEQ ID NO: 327, SEQ ID NO: 329, SEQ ID NO: 330, SEQ ID NO: 332 or SEQ ID NO: 333.

The antigen-binding molecules may comprise a light chain sequence. The light chain sequence may, for example, comprise or consist of a VL sequence listed in Table 3 that is directly fused to a CL sequence (e.g. SEQ ID NO: 320). For example, the light chain sequence may comprise an amino acid sequence having at least 70% sequence identity to SEQ ID NO: 328, SEQ ID NO: 331 or SEQ ID NO: 334.

In one embodiment, the present invention contemplates monovalent antigen-binding molecules produced by co-expression of a light chain, heavy chain and a truncated Fc domain. Suitably, the heavy chain incorporates hole mutations and P329G LALA mutations, while the truncated Fc domain incorporates knob mutations and P329G LALA mutations.

Expression of the antigen-binding molecule disclosed herein can be achieved for example in bacterial (e.g., Escherichia coli), yeast, insect or mammalian host cells upon cloning of the protein coding sequences of the constructs in the context of appropriate expression vectors with appropriate translational, transcriptional start sites, and, where appropriate, signal peptide sequences.

In one embodiment, the antigen-binding molecule is a multivalent antigen-binding molecule, non-limiting examples of which include: immunoglobulins, F(ab′)₂, tandem scFv (taFv or scFv₂), scFv-Fc, diabody, dAb₂/V_{H}H₂, minibodies, ZIP miniantibodies, barnase-barstar dimer, knobs-into-holes derivatives, SEED-IgG, heteroFc-scFv, Fab-scFv, Fab)₂/sc(Fab)₂, scFv-(TNFα)₃, scFv-Jun/Fos, Fab′-Jun/Fos, tribody, trimerbody, tribi-minibody, barnase-barstar trimer, collabody, DNL-F(ab)₃, scFv₃-C_{H}1/C_{L}, Fab-scFv₂, IgG-scFab, IgG-scFv, scFv-IgG, scFv₂-Fc, F(ab′)₂-scFv₂, scDB-Fc, scDb-C_{H}3, db-Fc, scFv₂-H/L, DVD-Ig, tandAb, scFv-dhlx-scFv, dAb₂-IgG, dAb-IgG, dAb-Fc-dAb, tetrabody, streptabody (scFv-streptavidin)₄, (scFv-p53)₄, [sc(Fv)₂]₂; tandem diabody (tandab) and combinations thereof.

In one embodiment, the multivalent antigen-binding molecule is selected from IgG-like antibodies (e.g., triomab/quadroma, Trion Pharma/Fresenius Biotech; knobs-into-holes, Genentech; CrossMAbs, Roche; electrostatically matched antibodies, AMGEN; LUZ-Y, Genentech; strand exchange engineered domain (SEED) body, EMD Serono; biolonic, Merus; and Fab-exchanged antibodies, Genmab), symmetric IgG-like antibodies (e.g., dual targeting (DT)-Ig, GSK/Domantis; two-in-one antibody, Genentech; crosslinked MAbs, karmanos cancer center; MAb₂, F-star; and Coy X-body, Coy X/Pfizer), IgG fusions (e.g., dual variable domain (DVD)-Ig, Abbott; IgG-like bispecific antibodies, Eli Lilly; Ts2Ab, Medimmune/AZ; BsAb, ZymoGenetics; HERCULES, Biogen Idec; TvAb, Roche) Fc fusions (e.g., scFv/Fc fusions, Academic Institution; SCORPION, Emergent BioSolutions/Trubion, ZymoGenetics/BMS; dual affinity retargeting technology (Fc-DART), MacroGenics; dual (ScFv)₂-Fab, National Research Center for Antibody Medicine) Fab fusions (e.g., F(ab)₂, Medarex/AMGEN; dual-action or Bis-Fab, Genentech; Dock-and-Lock (DNL), ImmunoMedics; bivalent bispecific, Biotechnol; and Fab-Fv, UCB-Celltech), ScFv- and diabody-based antibodies (e.g., bispecific T cell engagers (BiTEs), Micromet; tandem diabodies (Tandab), Affimed; DARTs, MacroGenics; Single-chain diabody, Academic; TCR-like antibodies, AIT, Receptor Logics; human serum albumin scFv fusion, Merrimack; and COMBODIES, Epigen Biotech), IgG/non-IgG fusions (e.g., immunocytokins, EMDSerono, Philogen, ImmunGene, ImmunoMedics; superantigen fusion protein, Active Biotech; and immune mobilizing mTCR Against Cancer, ImmTAC) and oligoclonal antibodies (e.g., Symphogen and Merus).

In one embodiment, the antibody is a bispecific or trispecific antibody. In one embodiment, the antibody is a bispecific antibody. The bispecific antibody may be one which comprises a first antigen-binding site that specifically binds ALPPL2 and a second antigen-binding site that specifically binds CD3. In one embodiment, the bispecific antibody is capable of binding to the cancer cell and recruit immune effector cells (e.g. T-cells) to kill the cancer cell. Antigen binding polypeptides that specifically binds CD3 are well known in the art. The second antigen-binding site may, for example, comprise CD3-specific CDR sequences or VH/VL sequences from Muromonab (Orthoclone OKT3), Foralumab, Teplizumab, Blinatumomab or Visilizumab. The bispecific antibody may, for example, comprise the VH CDR sequences of SEQ ID NO: 335-337 and the VL CDR sequences of SEQ ID NO: 338-340. Alternatively, the antibody may comprise the VH CDR sequences of SEQ ID NO: 341-343 and VL CDR sequences of SEQ ID NO: 344-346.

In one embodiment, bispecific antibodies of the invention are formed using a “protuberance-into-cavity” strategy, also referred to as “knobs into holes” that serves to engineer an interface between a first and second polypeptide for hetero-oligomerization.

The preferred interface comprises at least a part of the CH3 domain of an antibody constant domain. The “knobs into holes” mutations in the CH3 domain of an Fe sequence has been reported to greatly reduce the formation of homodimers (See, for example, Merchant et al., 1998, Nature Biotechnology, 16:677-681). “Protuberances” are constructed by replacing small amino acid side chains from the interface of the first polypeptide with larger side chains (e.g. tyrosine or tryptophan). Compensatory “cavities” of identical or similar size to the protuberances are optionally created on the interface of the second polypeptide by replacing large amino acid side chains with smaller ones (e.g. alanine or threonine). Where a suitably positioned and dimensioned protuberance or cavity exists at the interface of either the first or second polypeptide, it is only necessary to engineer a corresponding cavity or protuberance, respectively, at the adjacent interface. The protuberance and cavity can be made by synthetic means such as altering the nucleic acid encoding the polypeptides or by peptide synthesis. For further description of knobs into holes, see U.S. Pat. Nos. 5,731,168; 5,807,706; 5,821,333.

A general method of preparing a heteromultimer using the “protuberance-into-cavity” strategy comprises expressing, in one or separate host cells, a polynucleotide encoding a first polypeptide that has been altered from an original polynucleotide to encode a protuberance, and a second polynucleotide encoding a second polypeptide that has been altered from the original polynucleotide to encode the cavity. The polypeptides are expressed, either in a common host cell with recovery of the heteromultimer from the host cell culture, or in separate host cells, with recovery and purification, followed by formation of the heteromultimer. In some embodiments, the heteromultimer formed is a multimeric antibody, for example a bispecific antibody.

Chimeric Molecule

Disclosed herein is a chimeric molecule comprising an antigen-binding molecule as defined herein and a heterologous moiety.

In one embodiment, the heterologous moiety is a detectable moiety, a half-life extending moiety, or a therapeutic moiety.

Detectable moieties contemplated by the present invention include for example any species known in the art that is appropriate for diagnostic detection, including in vitro detection and in vivo imaging. The detectable moiety may be, for example, a fluorophore, a radionuclide reporter, a metal-containing nanoparticle or microparticle, an ultrasound contrast agent (e.g., a nanobubble or microbubble) or an optical imaging dye. This also includes contrast particles visible in magnetic resonance imaging (MRI) and magnetic particle imaging (MPI). Fluorophores can be detected and/or imaged, for example, by fluorescence polarization, fluorescence-activated cell sorting and fluorescence microscopy, which may or may not be in combination with electrospray ionization-mass spectrometry (ESI-MS) detection, as well as fluorescence emission computed tomography (FLECT) imaging. Radionuclide reporters can be detected and imaged by radionuclide (nuclear) detection, such as, for example, single-photon emission computed tomography (SPECT), positron emission tomography (PET) or scintigraphic imaging. Metal-containing nanoparticles or microparticles may be detected using optical imaging, including MRI, which is typically used with paramagnetic nanoparticles or microparticles, and MPI, which is generally used with superparamagnetic particles. Ultrasound contrast agents can be detected using ultrasound imaging including contrast-enhanced ultrasound (CEU).

The detectable label may also be an enzyme-substrate label. The enzyme may generally catalyze a chemical alteration of the chromogenic substrate that can be measured using various techniques. For example, the enzyme may catalyze a chemical alteration of the chromogenic substrate that can be measured using the various techniques. For example, the example may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light that can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, β-galactosidase, glucoamylase, lysozyme, saccharide oxidases (e.g., glucose oxidase, galactose oxidase, and glucose-6-phosphate dehydrogenase), heterocyclic oxidases (such as unease and xanthine oxidase), lactoperoxidase, microperoxidase, and the like.

Examples of enzyme-substrate combinations include, for example:

1) Horseradish peroxidase (HRPO) utilizes hydrogen peroxide to oxidize a dye precursor (e.g., orthophenylene diamine (OPD) or 3,3′,5,5′-tetramethyl benzidine hydrochloride (TMB));

2) alkaline phosphatase (AP) with para-Nitrophenyl phosphate as chromogenic substrate; and

3) β-D-galactosidase (β-D-Gal) with a chromogenic substrate (e.g., p-nitrophenyl-β-D-galactosidase) or fluorogenic substrate 4-methylumbelliferyl-β-D-galactosidase.

In another embodiment of the invention, the antigen-binding molecule need not be labeled, and the presence thereof can be detected using a labeled antibody which binds to the antigen-binding molecule. The antigen-binding molecule of the present invention may be employed in any known assay method, such as competitive binding assays, direct and indirect sandwich assays, immunohistochemistry and immunoprecipitation assays.

In one embodiment, the chimeric molecule comprises at least one heterologous moiety that is a “half-life extending moiety”. Half-life extending moieties, can comprise, for example, (i) XTEN polypeptides; (ii) Fc; (iii) albumin, (iv) albumin binding polypeptide or fatty acid, (v) the C-terminal peptide (CTP) of the 13 subunit of human chorionic gonadotropin, (vi) PAS; (vii) HAP; (viii) transferrin; (ix) polyethylene glycol (PEG); (x) hydroxyethyl starch (HES), (xi) polysialic acids (PSAs); (xii) a clearance receptor or fragment thereof which blocks binding of the chimeric molecule to a clearance receptor; (xiii) low complexity peptides; (xiv) or any combinations thereof. In some embodiments, the half-life extending moiety comprises an Fc region. In other embodiments, the half-life extending moiety comprises two Fc regions fused by a linker. Exemplary heterologous moieties also include, e.g., FcRn binding moieties (e.g., complete Fc regions or portions thereof which bind to FcRn), single chain Fc regions (scFc regions, e.g., as described in U.S. Publ. No. 20080260738, WO 2008/012543 and WO 2008/1439545), or processable scFc regions. In some embodiments, a heterologous moiety can include an attachment site for a non-polypeptide moiety such as polyethylene glycol (PEG), hydroxyethyl starch (HES), polysialic acid, or any derivatives, variants, or combinations of these moieties.

In some embodiments, at least one heterologous moiety is a therapeutic moiety. In certain embodiments, the therapeutic moiety is selected from an anti-cancer moiety (e.g., cytostatic/toxic, and/or anti-proliferative drugs), an immunotherapeutic moiety and an anti-inflammatory moiety. In some embodiments, the therapeutic agent is useful in the treatment of cancer. Useful classes of anti-cancer agents include chemotherapeutic agents, representative examples of which include antitubulin agents, auristatins, DNA minor groove binders, DNA replication inhibitors, alkylating agents (e.g., platinum complexes such as cis-platin, mono(platinum), bis(platinum) and tri-nuclear platinum complexes and carboplatin), anthracyclines, antibiotics, antifolates, antimetabolites, calmodulin inhibitors, chemotherapy sensitizers, duocarmycins, etoposides, fluorinated pyrimidines, ionophores, lexitropsins, maytansinoids, nitrosoureas, platinols, pore-forming compounds, purine antimetabolites, puromycins, radiation sensitizers, rapamycins, steroids, taxanes, topoisomerase inhibitors, vinca alkaloids, or the like.

In one embodiment, the therapeutic moiety is an auristatin such as monomethyl auristatin F (MMAF) or monomethyl auristatin E (MMAE).

In one embodiment, the antigen-binding molecule is joined to the therapeutic moiety via a valine-citrulline (Vc) linker.

Polynucleotides, Constructs and Host Cells

Disclosed herein is an isolated polynucleotide comprising a nucleic acid sequence encoding the antigen-binding molecule as defined herein, or the chimeric molecule as defined herein.

The term “polynucleotide” or “nucleic acid” are used interchangeably herein to refer to a polymer of nucleotides, which can be mRNA, RNA, cRNA, cDNA or DNA. The term typically refers to polymeric form of nucleotides of at least 10 bases in length, either ribonucleotides or deoxynucleotides or a modified form of either type of nucleotide. The term includes single and double stranded forms of DNA.

Also disclosed herein is a vector that comprises a nucleic acid encoding the antigen-binding molecule as described herein.

By “vector” is meant a nucleic acid molecule, preferably a DNA molecule derived, for example, from a plasmid, bacteriophage, or virus, into which a nucleic acid sequence may be inserted or cloned. A vector preferably contains one or more unique restriction sites and may be capable of autonomous replication in a defined host cell including a target cell or tissue or a progenitor cell or tissue thereof, or be integrable with the genome of the defined host such that the cloned sequence is reproducible. Accordingly, the vector may be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. A vector system may comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. The vector may also include a selection marker such as an antibiotic resistance gene that can be used for selection of suitable transformants. Examples of such resistance genes are well known to those of skill in the art.

Disclosed herein is a construct comprising a nucleic acid sequence encoding the antigen-binding molecule as defined herein, or the chimeric molecule as defined herein in operable connection with one or more control sequences.

The term “construct” refers to a recombinant genetic molecule including one or more isolated nucleic acid sequences from different sources. Thus, constructs are chimeric molecules in which two or more nucleic acid sequences of different origin are assembled into a single nucleic acid molecule and include any construct that contains (1) nucleic acid sequences, including regulatory and coding sequences that are not found together in nature (i.e., at least one of the nucleotide sequences is heterologous with respect to at least one of its other nucleotide sequences), or (2) sequences encoding parts of functional RNA molecules or proteins not naturally adjoined, or (3) parts of promoters that are not naturally adjoined. Representative constructs include any recombinant nucleic acid molecule such as a plasmid, cosmid, virus, autonomously replicating polynucleotide molecule, phage, or linear or circular single stranded or double stranded DNA or RNA nucleic acid molecule, derived from any source, capable of genomic integration or autonomous replication, comprising a nucleic acid molecule where one or more nucleic acid molecules have been operably linked. Constructs of the present invention will generally include the necessary elements to direct expression of a nucleic acid sequence of interest that is also contained in the construct, such as, for example, a target nucleic acid sequence or a modulator nucleic acid sequence. Such elements may include control elements or regulatory sequences such as a promoter that is operably linked to (so as to direct transcription of) the nucleic acid sequence of interest, and often includes a polyadenylation sequence as well. Within certain embodiments of the invention, the construct may be contained within a vector. In addition to the components of the construct, the vector may include, for example, one or more selectable markers, one or more origins of replication, such as prokaryotic and eukaryotic origins, at least one multiple cloning site, and/or elements to facilitate stable integration of the construct into the genome of a host cell. Two or more constructs can be contained within a single nucleic acid molecule, such as a single vector, or can be containing within two or more separate nucleic acid molecules, such as two or more separate vectors. An “expression construct” generally includes at least a control sequence operably linked to a nucleotide sequence of interest. In this manner, for example, promoters in operable connection with the nucleotide sequences to be expressed are provided in expression constructs for expression in an organism or part thereof including a host cell. For the practice of the present invention, conventional compositions and methods for preparing and using constructs and host cells are well known to one skilled in the art, see for example, Molecular Cloning: A Laboratory Manual, 3rd edition Volumes 1, 2, and 3. J. F. Sambrook, D. W. Russell, and N. Irwin, Cold Spring Harbor Laboratory Press, 2000.

By “control element”, “control sequence”, “regulatory sequence” and the like, as used herein, mean a nucleic acid sequence (e.g., DNA) necessary for expression of an operably linked coding sequence in a particular host cell. The control sequences that are suitable for prokaryotic cells for example, include a promoter, and optionally a cis-acting sequence such as an operator sequence and a ribosome binding site. Control sequences that are suitable for eukaryotic cells include transcriptional control sequences such as promoters, polyadenylation signals, transcriptional enhancers, translational control sequences such as translational enhancers and internal ribosome binding sites (IRES), nucleic acid sequences that modulate mRNA stability, as well as targeting sequences that target a product encoded by a transcribed polynucleotide to an intracellular compartment within a cell or to the extracellular environment.

Disclosed herein is a host cell that contains the construct as defined herein.

The terms “host”, “host cell”, “host cell line” and “host cell culture” are used interchangeably and refer to cells into which exogenous nucleic acid has been introduced, including the progeny of such cells. Host cells include “transformants” and “transformed cells”, which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell is any type of cellular system that can be used to generate the antigen-binding molecules of the present invention. Host cells include cultured cells, e.g., mammalian cultured cells, such as CHO cells, BHK cells, NS0 cells, SP2/0 cells, YO myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue.

Pharmaceutical Composition

Disclosed herein is a pharmaceutical composition comprising the antigen-binding molecule as defined herein, or the chimeric molecule as defined herein, and a pharmaceutically acceptable carrier.

By “pharmaceutically acceptable carrier” is meant a pharmaceutical vehicle comprised of a material that is not biologically or otherwise undesirable, i.e., the material may be administered to a subject along with the selected active agent without causing any or a substantial adverse reaction. Carriers may include excipients and other additives such as diluents, detergents, coloring agents, wetting or emulsifying agents, pH buffering agents, preservatives, and the like.

Representative pharmaceutically acceptable carriers include any and all solvents, dispersion media, coatings, surfactants, antioxidants, preservatives {e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient(s), its use in the pharmaceutical compositions is contemplated.

The pharmaceutical compositions may be in a variety of forms. These include, for example, liquid, semi-solid and solid dosage forms, such as liquid solutions (e.g., injectable and infusible solutions), dispersions or suspensions, liposomes and suppositories. The preferred form depends on the intended mode of administration and therapeutic application. Suitable pharmaceutical compositions may be administered intravenously, subcutaneously or intramuscularly. In some embodiments, the compositions are in the form of injectable or infusible solutions. A preferred mode of administration is parenteral (e.g., intravenous, subcutaneous, intraperitoneal, intramuscular). In specific embodiments, the pharmaceutical composition is administered by intravenous infusion or injection. In other embodiments, the pharmaceutical composition is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject invention, pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives can also be present such as for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin and/or by the maintenance of the required particle size. In specific embodiments, an agent of the present disclosure may be conjugated to a vehicle for cellular delivery. In these embodiments, the agent may be encapsulated in a suitable vehicle to either aid in the delivery of the agent to target cells, to increase the stability of the agent, or to minimize potential toxicity of the agent. As will be appreciated by a skilled artisan, a variety of vehicles are suitable for delivering an agent of the present disclosure. Non-limiting examples of suitable structured fluid delivery systems may include nanoparticles, liposomes, microemulsions, micelles, dendrimers and other phospholipid-containing systems. Methods of incorporating agents of the present disclosure into delivery vehicles are known in the art. Although various embodiments are presented below, it will be appreciate that other methods known in the art to incorporate an antigen-binding molecule, as described herein, into a delivery vehicle are contemplated.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. An antigen-binding molecule of the present disclosure can be administered on multiple occasions. Intervals between single dosages can be daily, weekly, monthly or yearly. Intervals can also be irregular as indicated by measuring blood levels of modified polypeptide or antigen in the patient. Alternatively, the antigen-binding molecule can be administered as a sustained release formulation, in which case less frequent administration is required. Dosage and frequency vary depending on the half-life of the polypeptide in the patient.

It may be advantageous to formulate compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutically acceptable carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

Dosages and therapeutic regimens of the antigen-binding molecule can be determined by a skilled artisan. In certain embodiments, the antigen-binding molecule is administered by injection (e.g., subcutaneously or intravenously) at a dose of about 0.01 to 50 mg/kg, e.g., 0.01 to 0.1 mg/kg, e.g., about 0.1 to 1 mg/kg, about 1 to 5 mg/kg, about 5 to 25 mg/kg, about 10 to 50 mg/kg. The dosing schedule can vary from e.g., once a week to once every 2, 3, or 4 weeks.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

Methods of Treatment

Provided herein is a method for reducing the expression or activity of ALPPL2 in a cell (such as a cancer cell). Provided herein is a method for reducing the expression or activity of ALPPL2 in a cancer cell, the method comprising contacting the cancer cell with an antigen-binding molecule, a chimeric molecule, a polynucleotide, a construct, a vector, a host cell or a pharmaceutical composition as defined herein.

Disclosed herein is a method for reducing the expression or activity of ALPPL2 in a cancer cell, the method comprising contacting the cancer cell with an antigen-binding molecule as defined herein or a chimeric molecule as defined herein.

The term “tumor,” as used herein, refers to any neoplastic cell growth and proliferation, whether malignant or benign, and all pre-cancerous and cancerous cells and tissues. The terms “cancer” and “cancerous” refer to or describe the physiological condition in mammals that is typically characterized in part by unregulated cell growth. As used herein, the term “cancer” refers to non-metastatic and metastatic cancers, including early stage and late stage cancers. The term “precancerous” refers to a condition or a growth that typically precedes or develops into a cancer. By “non-metastatic” is meant a cancer that is benign or that remains at the primary site and has not penetrated into the lymphatic or blood vessel system or to tissues other than the primary site. Generally, a non-metastatic cancer is any cancer that is a Stage 0, I, or II cancer, and occasionally a Stage III cancer. By “early stage cancer” is meant a cancer that is not invasive or metastatic or is classified as a Stage 0, I, or II cancer. The term “late stage cancer” generally refers to a Stage III or Stage IV cancer, but can also refer to a Stage II cancer or a substage of a Stage II cancer. One skilled in the art will appreciate that the classification of a Stage II cancer as either an early stage cancer or a late stage cancer depends on the particular type of cancer. Illustrative examples of cancer include, but are not limited to, breast cancer, prostate or testicular cancer, ovarian cancer, cervical cancer, pancreatic cancer, colorectal cancer, lung cancer, hepatocellular cancer, gastric cancer, liver cancer, bladder cancer, cancer of the urinary tract, thyroid cancer, renal cancer, carcinoma, melanoma, brain cancer, non-small cell lung cancer, squamous cell cancer of the head and neck, endometrial cancer, multiple myeloma, rectal cancer, mesothelioma, endometrial cancer and esophageal cancer. In an exemplary embodiment, the cancer is colorectal, endometrial, gastric, mesothelioma, ovarian, pancreatic or testicular cancer.

Provided herein is a method for reducing or inhibiting proliferation, survival and viability of a tumor in a subject, the method comprising administering an antigen-binding molecule, a chimeric molecule, a polynucleotide, a construct, a vector, a host cell or a pharmaceutical composition as defined herein to the subject.

Disclosed herein is a method for reducing or inhibiting proliferation, survival and viability of a tumor in a subject, the method comprising administering an antigen-binding molecule as defined herein or a chimeric molecule as defined herein to the subject.

The term “patient” includes human and other mammalian subjects that receive either prophylactic or therapeutic treatment. As used herein, the term “subject” includes any human or non-human animal. For example, the methods of the present invention can be used to treat a subject having cancer. In one embodiment, the subject is a human. The term “non-human animal” includes all vertebrates, e.g., mammals and non-mammals, such as non-human primates, sheep, dog, cow, chickens, amphibians, reptiles, etc. For example, the methods of the present invention can be used to treat a subject having cancer. In one embodiment, the subject is a human. The term “non-human animal” includes all vertebrates, e.g., mammals and non-mammals, such as non-human primates, sheep, dog, cow, chickens, amphibians, reptiles, etc.

The methods as disclosed herein may comprises the administration of a “therapeutically effective amount” of an agent (e.g. an antigen-binding molecule, a chimeric molecule, a polynucleotide, a construct, a vector, a host cell or a pharmaceutical composition) to a subject. As used herein the term “therapeutically effective amount” includes within its meaning a non-toxic but sufficient amount of an agent or compound to provide the desired therapeutic effect. The exact amount required will vary from subject to subject depending on factors such as the species being treated, the age and general condition of the subject, the severity of the condition being treated, the particular agent being administered and the mode of administration and so forth. Thus, it is not possible to specify an exact “effective amount”. However, for any given case, an appropriate “effective amount” may be determined by one of ordinary skill in the art using only routine experimentation.

In one embodiment, there is provided a method of treating cancer in a subject, wherein the method comprises administering an antigen-binding molecule, a chimeric molecule, a polynucleotide, a construct, a vector, a host cell or a pharmaceutical composition as defined herein to the subject

Disclosed herein is a method of treating cancer in a subject, wherein the method comprises administering an antigen-binding molecule as defined herein or a chimeric molecule as defined herein to the subject.

The term “treating” as used herein may refer to (1) preventing or delaying the appearance of one or more symptoms of the disorder; (2) inhibiting the development of the disorder or one or more symptoms of the disorder; (3) relieving the disorder, i.e., causing regression of the disorder or at least one or more symptoms of the disorder; and/or (4) causing a decrease in the severity of one or more symptoms of the disorder.

In one embodiment, the cancer is gastric, ovarian or pancreatic cancer.

Disclosed herein is an antigen-binding molecule, a chimeric molecule, a polynucleotide, a construct, a vector, a host cell or a pharmaceutical composition as defined herein for use as a medicament.

Disclosed herein is an antigen-binding molecule as defined herein or a chimeric molecule as defined herein for use in the treatment of cancer.

Disclosed herein is the use of an antigen-binding molecule, a chimeric molecule, a polynucleotide, a construct, a vector, a host cell or a pharmaceutical composition in the manufacture of a medicament for the treatment of a subject in need. The subject may be a subject suffering from cancer.

Disclosed herein is the use of an antigen-binding molecule as defined herein or a chimeric molecule as defined herein in the manufacture of a medicament for the treatment of cancer.

Disclosed herein is a method of treating a disorder or condition associated with the undesired expression of ALPPL2 in a subject, wherein the method comprises administering an antigen-binding molecule a chimeric molecule, a polynucleotide, a construct, a vector, a host cell or a pharmaceutical composition as defined herein to the subject.

Disclosed herein is a method of treating a disorder or condition associated with the undesired expression of ALPPL2 in a subject, wherein the method comprises administering an antigen-binding molecule as defined herein or a chimeric molecule as defined herein to the subject.

In one embodiment, the disorder or condition associated with the undesired expression of ALPPL2 is a cancer.

In one embodiment, the cancer is a solid cancer.

In one embodiment, the cancer is cervical, colon, endometrial, gastric, ovarian or pancreatic cancer.

Kits

Disclosed herein is a kit for detecting cancer, the kit comprising an antigen-binding molecule or a chimeric molecule as defined herein

Methods of Diagnosis

Disclosed herein is a method of determining the likelihood of a cancer in a subject, wherein the method comprises detecting ALPPL2 in a sample obtained from the subject, wherein an increased level of ALPPL2 in the sample as compared to a reference indicates the likelihood of cancer in the subject.

In one embodiment, the method comprises detecting ALPPL2 with an antigen-binding molecule as defined herein or a chimeric molecule as defined herein.

Disclosed herein is a method of treating a cancer in a subject, wherein the method comprises a) detecting ALPPL2 in a sample obtained from the subject, wherein an increased level of ALPPL2 in the sample as compared to a reference indicates an increased likelihood of cancer in the subject; and b) treating a subject found to have an increased likelihood of cancer.

In one embodiment, the method comprises detecting ALPPL2 with an antigen-binding molecule as defined herein or a chimeric molecule as defined herein.

In one embodiment, the method comprises treating the subject with an antigen-binding molecule as defined herein or a chimeric molecule as defined herein.

Disclosed herein is a method of identifying a subject who is likely to be responsive to treatment with an anti-ALPPL2 antibody, the method comprising detecting ALPPL2 in a sample obtained from the subject, wherein an increased level of ALPPL2 indicates that the subject is likely to be responsive to treatment with the ALPPL2 antibody.

In one embodiment, the method comprises detecting ALPPL2 with an antigen-binding molecule as defined herein or a chimeric molecule as defined herein.

Disclosed herein is a method of identifying and treating a subject who is likely to be responsive to treatment with an anti-ALPPL2 antibody, the method comprising a) detecting ALPPL2 in a sample obtained from the subject, wherein an increased level of ALPPL2 indicates that the subject is likely to be responsive to treatment with the ALPPL2 antibody; and b) treating the subject found likely to be responsive to treatment with the ALPPL2 antibody.

As used herein, “and/or” refers to and encompasses any and all possible combinations of one or more of the associated listed items, as well as the lack of combinations when interpreted in the alternative (or).

As used in this application, the singular form “a,” “an,” and “the” include plural references unless the context clearly dictates otherwise. For example, the term “an agent” includes a plurality of agents, including mixtures thereof.

By “about” is meant a quantity, level, value, number, frequency, percentage, dimension, size, amount, weight or length that varies by as much 15, 14, 13, 12, 11, 10, 9, 8, 7, 6, 5, 4, 3, 2 or 1% to a reference quantity, level, value, number, frequency, percentage, dimension, size, amount, weight or length.

Throughout this specification and the statements which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Those skilled in the art will appreciate that the invention described herein in susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

Certain embodiments of the invention will now be described with reference to the following examples which are intended for the purpose of illustration only and are not intended to limit the scope of the generality hereinbefore described.

### Examples

Target ID and Background

Gastric cancer is an East Asia prevalent disease, in which 79% of patients are diagnosed at stage IV with a five-year survival rate is less than 5%. A novel cell surface biomarker, ALPPL2, was identified as a target for therapeutic antibodies and companion diagnostics. Biomarker identification was performed on RNA-sequencing data from 19 gastric cancer patients through rigorous bioinformatics analysis.

ALPPL2 protein expression was validated in 6 gastric cancer cell lines using a commercial anti-ALPPL2 antibody in immunohistochemical staining. Strong membraneous staining was observed in gastric cancer cell lines overexpressing ALPPL2 mRNA while no obvious staining was seen in cell lines which do not overexpress ALPPL2 transcript. Clinical prevalence was also assessed by immunohistochemical staining of 2 gastric tumour microarrays. A total of 198 tumour cores of various stages of the disease and different regions of the stomach were stained. The results indicate that 32 out of 198 cases showed ALPPL2 membranous staining which amounts to 16%. No obvious membranous staining was observed in both adjacent matched and unmatched normal tissues.

Antibody Generation

Antibodies against human ALPPL2 were generated by immunizing rabbits with the antigen. The rabbit antibodies were isolated by cloning the antibody genes directly from rabbit single B cells.

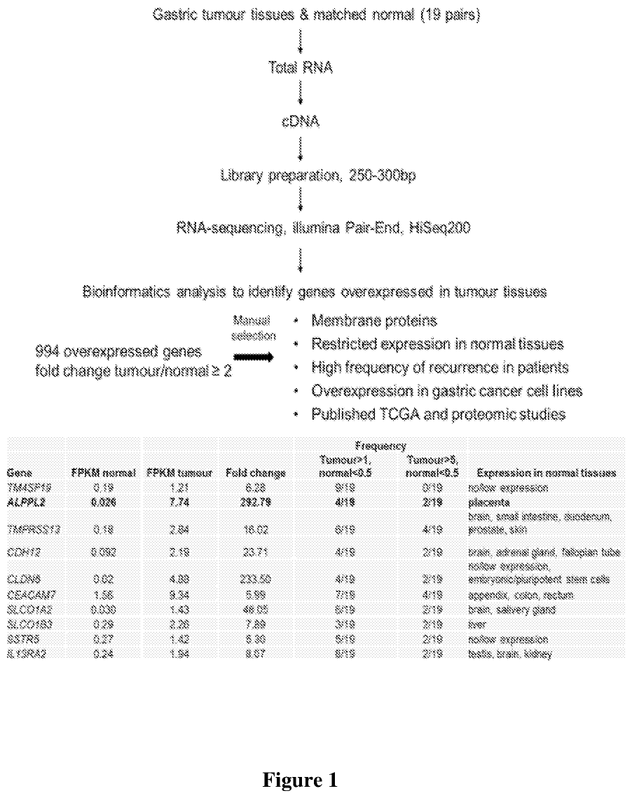

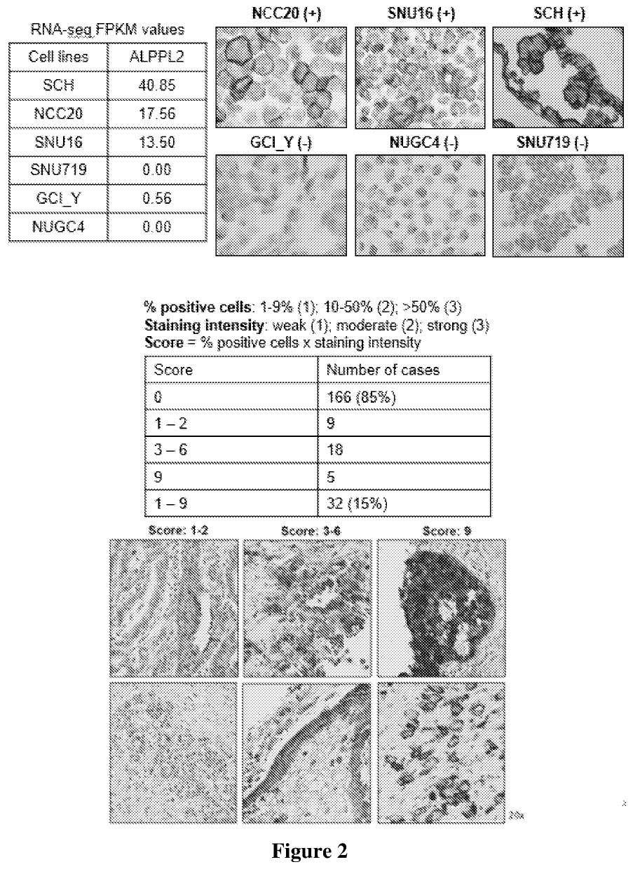

During the screening process, clones that bind to ALPPL2 but not the related ALPI, which is expressed in normal intestinal tissue, were selected (FIGS. 1 and 2). In total 36 clones with high affinity to human ALPP/ALPPL2 were isolated. The amino acid sequences of the variable regions and complementarity determining regions are shown in Table 1 and 2.

Affinity and Specificity

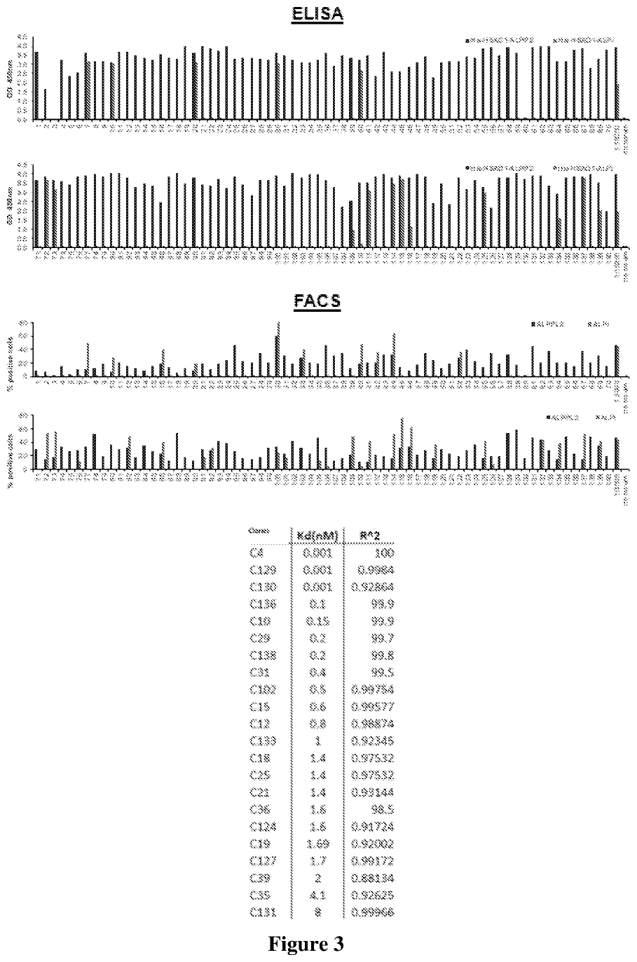

Specific clones were screened and identified by ELISA and high throughput flow cytometry (FIG. 3). Rabbit kidney cells were transfected with either truncated (for ELISA screen) or full-length (for FACS screen) ALPI and ALPPL2.

A subset of clones with specificity towards ALPPL2/ALPP but not ALPI were selected further for affinity measurement using Biolayer Interferometry analysis (FIG. 3). In this technique, single concentration of different supernatant clones from rabbit B cells were immobilized on protein A biosensors. The biosensors were then incubated with analyte to measure affinity.

A comparable humanized monoclonal antibody disclosed in a prior art was engineered by grafting the CDR to the same framework and to evaluate binding to both ALPPL2 and ALPI by ELISA. The comparable humanized monoclonal antibody has the following V_{H} and V_{L} sequences:

| V_{H} | V_{L} |
|---|---|
| QVQLQQSGGGLVKPGGS | QSALTQPASVSGSPGQSI |
| LRLSCAASGFTFSSYDM | TISCTGTSSDVGGYNYVS |
| HWVRQAPGKGLEWVAVI | WYQQHPGKAPKVMIYDVT |
| SYDGSNKYYADSVKGRF | NRPSGVSNRFSGSKSGNT |
| TISRDNSKNTLYLQMDS | ASLTISGLQAEDEADYYC |
| LRAEDTAVYFCAKEGDS | SSYTSTSTLVVFGGGTKL |
| SRWSYDLWGRGTLVTVS | TVLG |
| S | (SEQ ID NO: 325) |
| (SEQ ID NO: 324) | |

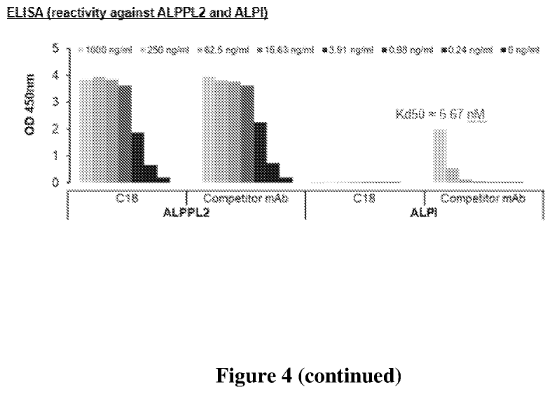

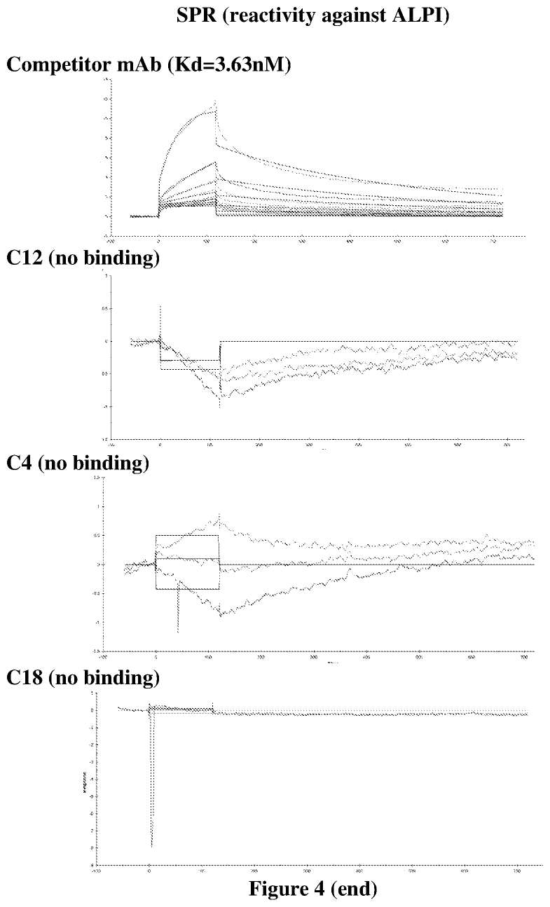

The gene was synthesized and cloned into the expression vector for recombinant antibody production. The surface plasmon reasonance data in FIG. 4 shows that the antibody disclosed in prior art exhibits non-specific binding to ALPI but not the antibodies of the present invention.

Immunohistochemistry (IHC) Activity,

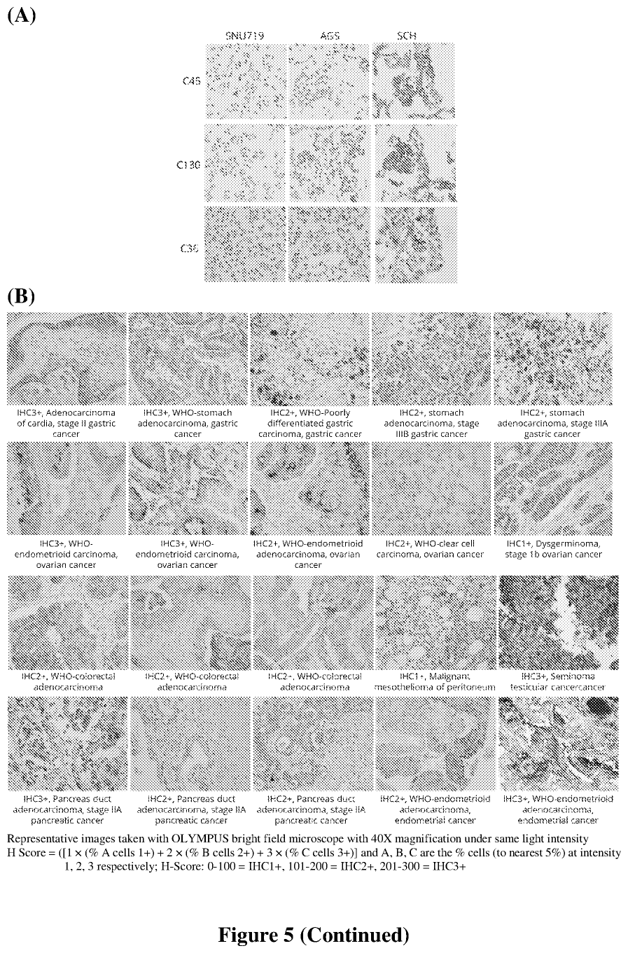

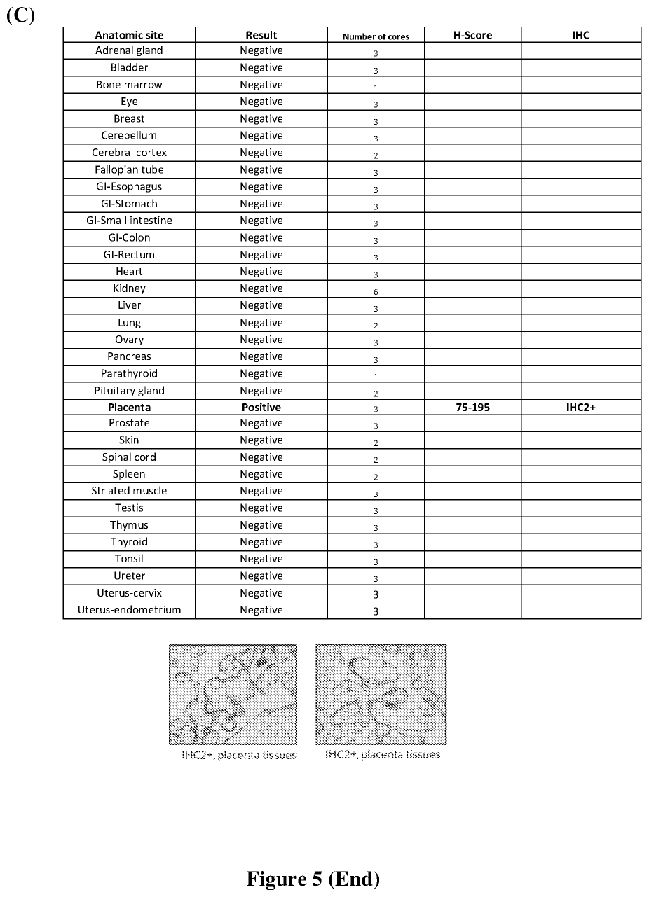

To enable development of a companion diagnostic, the IHC activity of the antibodies was evaluated (FIG. 5). Antigen retrieval by Proteinase-K digestion, but not heat mediated antigen retrieval, enabled detection. C36, C45 and C130 enabled detection in ALPPL2+ve cell lines (SCH) and formalin fixed paraffin embedded (FFPE) human tumor tissues by IHC. This shows that the antibodies may have diagnostic applications in patient stratification and therapeutic applications for the treatment of ALPPL2/ALPP+ve tumors, including gastric cancer, ovarian cancer, colorectal cancer, pancreatic cancer, endometrial cancer, mesothelioma and testicular cancer. C36 shows negative staining in all normal tissues except placenta, suggesting ALPPL/ALPP has no/low expression in normal tissues. This is also indicative the optimal therapeutic window of these antibodies in the clinic.

Non-Human Primate (NHP) Cross-Reactivity

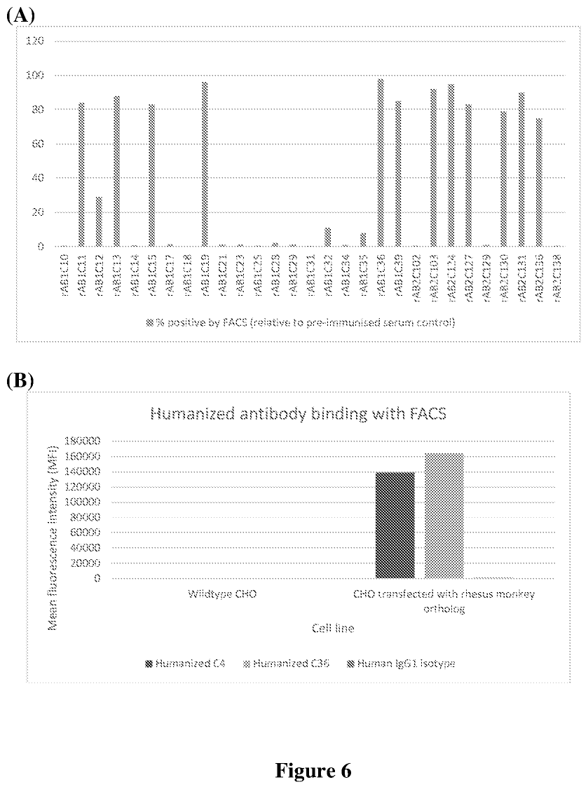

The antibodies were further evaluated for cross-reactivity to non-human primate orthologs (FIG. 6). Select clones showed reactivity to rhesus macaque ortholog.

Humanized Clones (Affinity, Selectivity and Specificity)

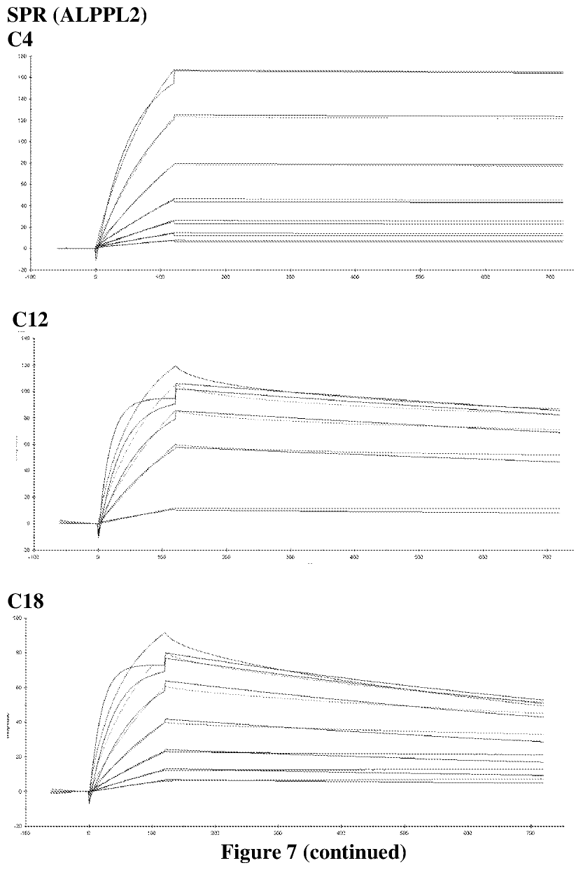

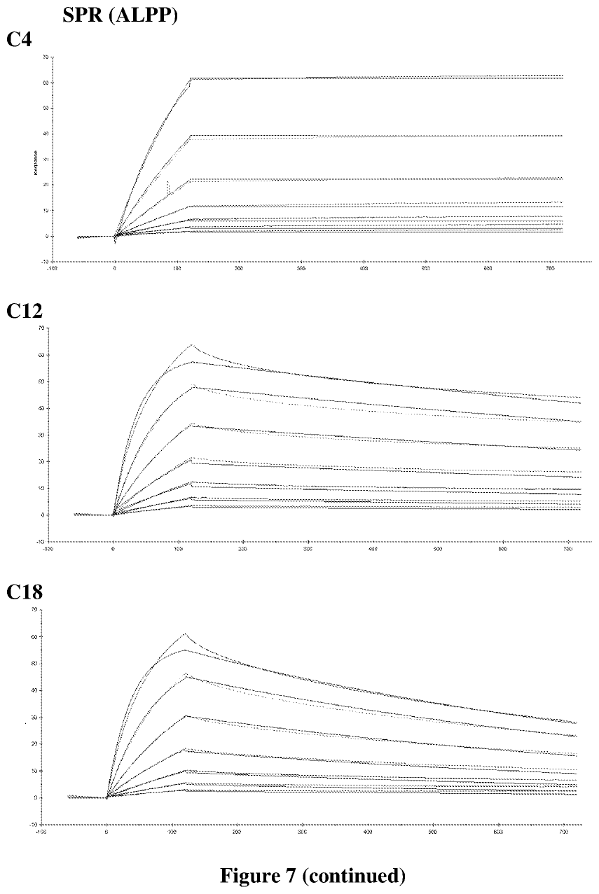

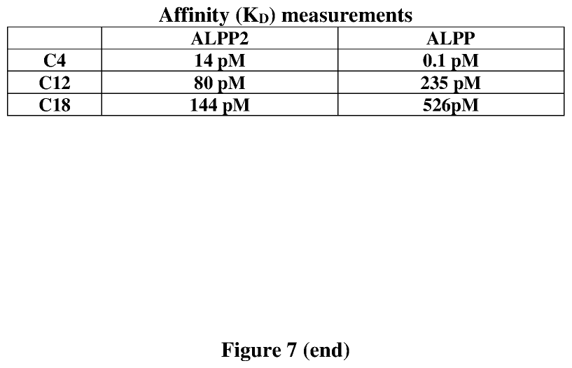

Select clones (C4, C15, C131, C12, C18, C36 and C53) were humanized by grafting the CDRs to a human IgG1 framework. These humanized clones were shown to retain high ALPP/ALPPL2 affinity using surface plasmon reasonance (FIG. 7). Surface plasmon resonance was studied using Biacore T200. Ligands (e.g. ALPPL2 or ALPP) were immobilized on biosensors CM5 chips captured with the streptavidin. Ligands-loaded sensors were then incubated with different concentrations of analyte (recombinant expressed humanized antibody clones) to measure affinity.

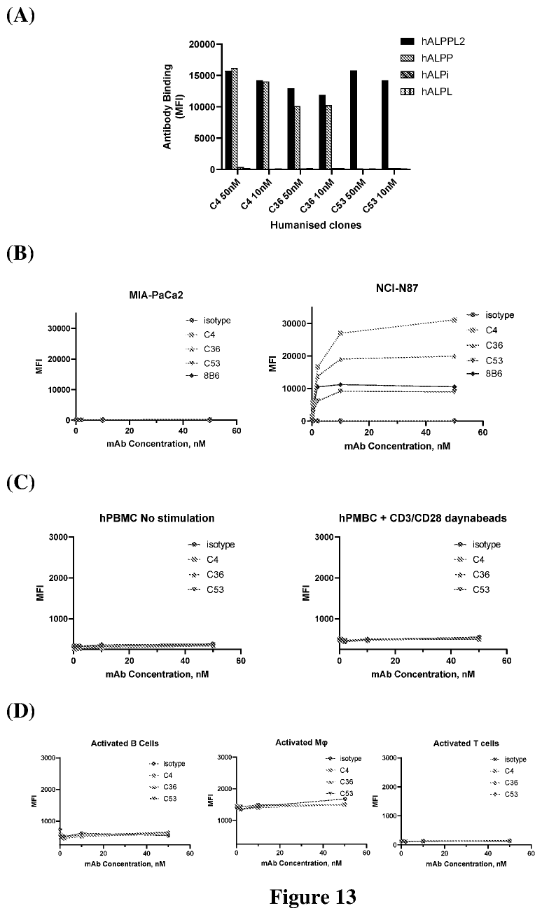

Humanized clones (C4, C36 and C53) maintained the selectivity towards cancer-specific ALPPL2 and/or ALPP, but not towards the widely expressed ALPI and ALPL (FIG. 13). Humanized clones were also specific to the cancer cells but not the normal naïve and stimulated immune cells (FIG. 13).

Humanized Clones (ADCC)

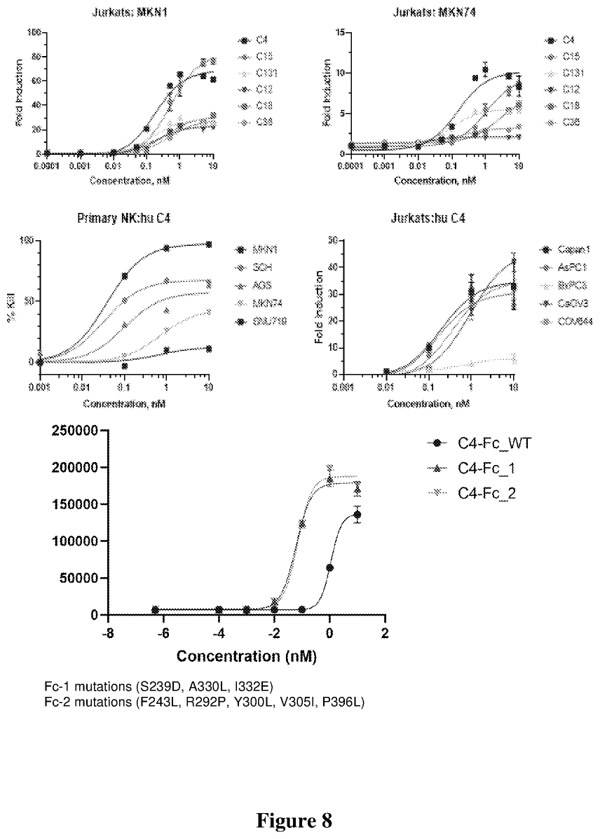

The therapeutic efficacy of the humanized clones was tested by first evaluating antibody-dependent cellular toxicity by co-culture of reporter or primary NK cells with cancer cell lines (FIG. 8). C4 resulted in the most potent ADCC induction in both gastric cancer cell lines with high and low target expression, when compared to the other clones. The potency of C4 was confirmed in a co-culture assay of primary NK cells with different gastric cancer cell lines. C4 achieved near complete killing of a high expressing cell line, and showed potency (maximum % kill and EC50) that was proportional to the target expression level. In addition, C4 was also tested in a reporter assay and induction of ADCC with different ovarian and pancreatic cancer cell lines was confirmed.

The Fc region of humanized C4 was further engineered to enhance ADCC. Reporter assay confirmed more the humanized C4 with engineered Fc more potently induced ADCC with a gastric cancer cell line

Humanized Clones (ADC)

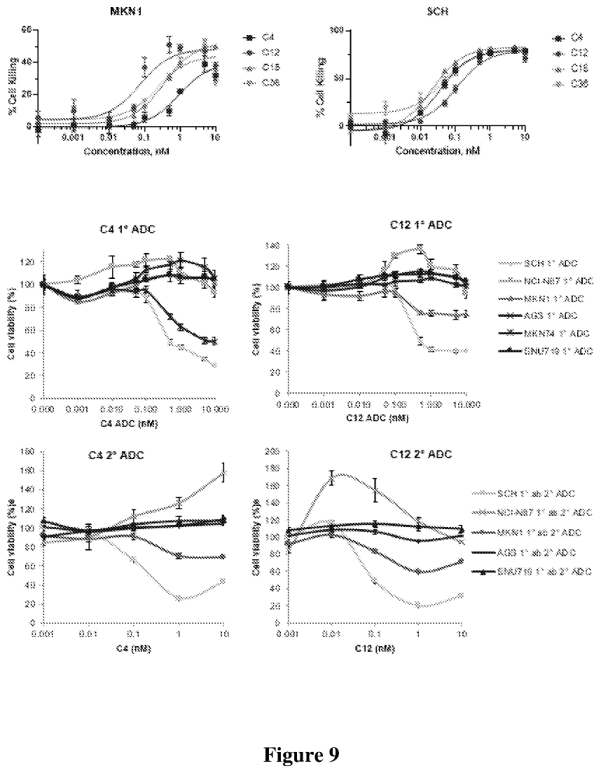

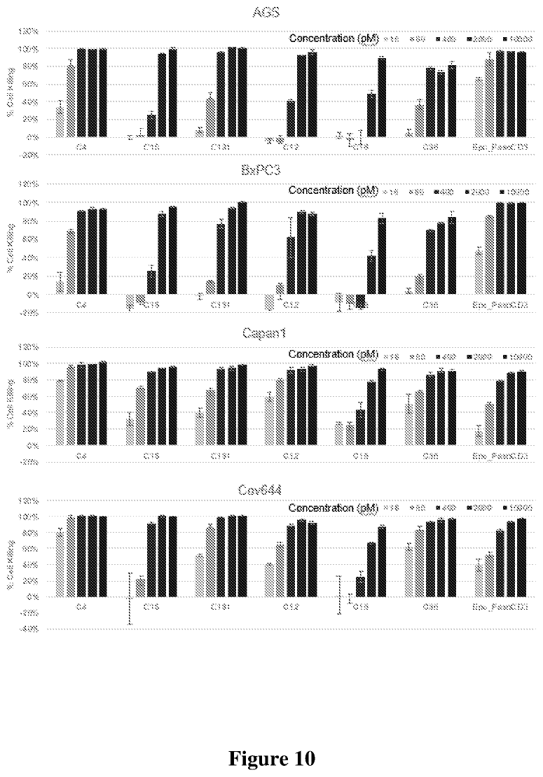

The suitability of using these humanized antibodies as antibody-drug conjugate was evaluated. First, killing of gastric cancer cell lines by these antibodies was tested in the presence of secondary antibodies conjugated with vc-MMAF (FIG. 9). Secondary ADC assay revealed some differences in potency among the clones; killing was only seen in select gastric cancer cell lines with high target expression but not others. These results were confirmed using the same antibodies conjugated with vc-MMAE. Primary conjugation of the antibodies with vc-MMAE potentiated killing of the same gastric cancer cell lines observed in the secondary ADC assay.

Humanized Clones (T-cell engagers)

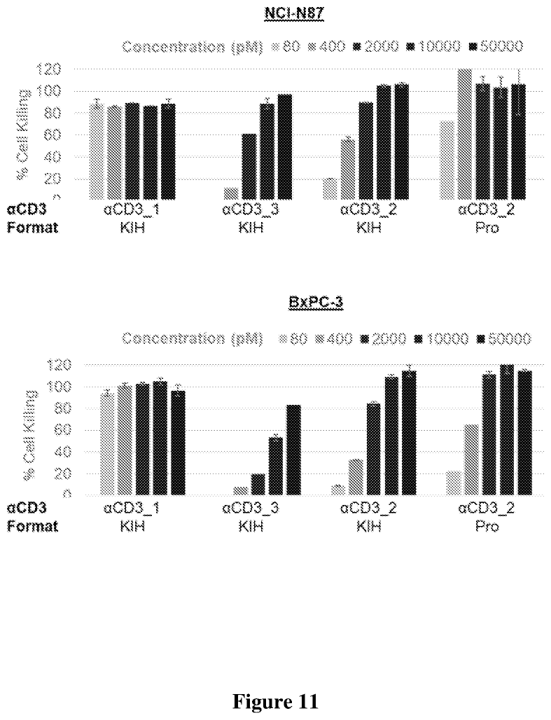

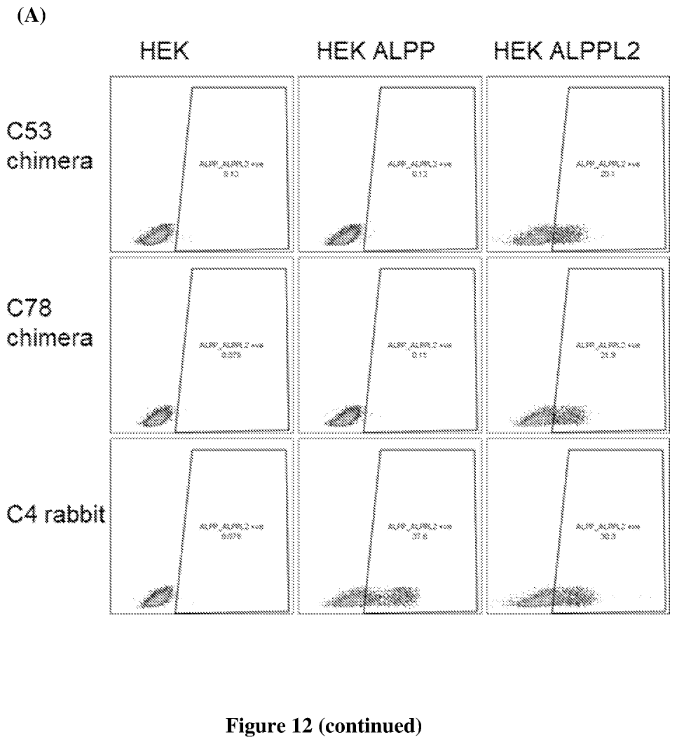

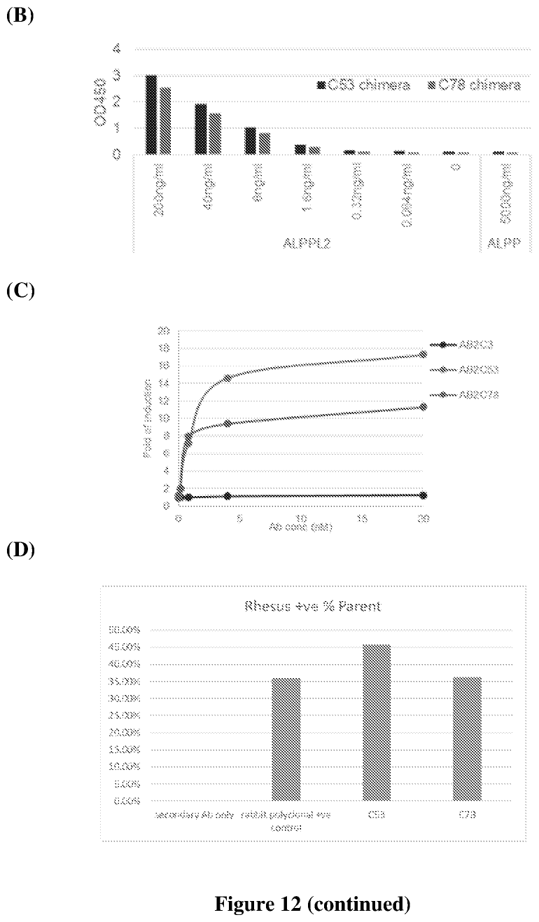

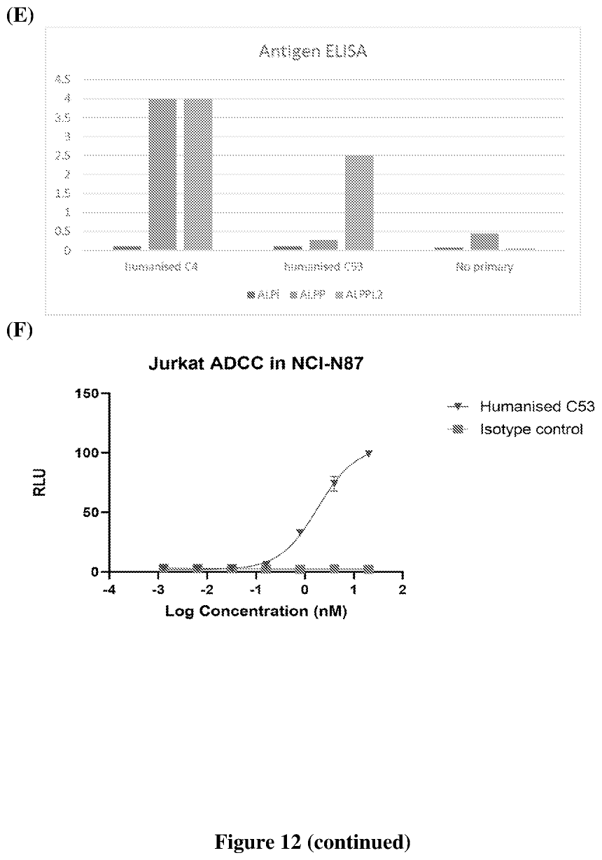

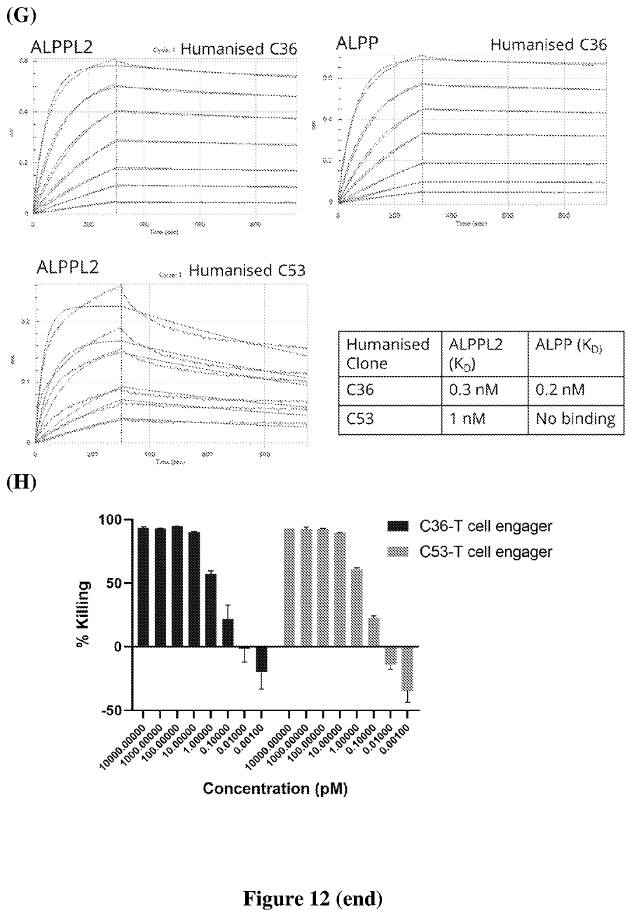

It was further exemplified that these humanized antibodies can be successfully adapted for use as T-cell engagers to induce potent T-cell mediated killing of cancer cells (FIG. 10, FIG. 11 and FIG. 12). Bispecific antibodies by heterodimerisation of the humanized clones with anti-CD3 antibodies achieved potent and specific killing of gastric, ovarian and pancreatic cancer cell lines irrespective of target expression level. In particular, C4 achieved near-complete killing of multiple cancer cell lines at pM concentrations.

ALPPL2 Specific Clone

Chimerized and humanized C53 clone demonstrated binding specificity towards ALPPL2 but not ALPP (FIG. 12). Chimerized C53 is also cross-reactive towards rhesus macaque ortholog. Binding affinity of humanized C53 as compared to humanized C36 to ALPPL and ALPP was performed using Biolayer Interferometry. In this technique, biotinylated ligands (i.e. ALPPL2 or ALPP) were immobilized on SA biosensors. Ligands-loaded sensors were then incubated with different concentrations of analyte (recombinantly expressed humanized antibody clones) in the buffer. Humanized C53 antibody demonstrated nM binding affinity towards ALPPL but not ALPP as determined Biolayer Interferometry, whereas humanized C36 demonstrated similar binding affinity towards ALPPL2 and ALPP.

It was further exemplified that humanized C53 antibody maintained its killing potency in both ADCC and adapted for use as T-cell engager to induce potent T-cell mediated killing of cancer cells.

1. An antigen-binding molecule that specifically binds ALPPL2 and/or ALPP but not ALPL or ALPI, comprising:
a) a heavy chain variable region (V_{H}) comprising VHCDR1, VHCDR2 and VHCDR3 amino acid sequences; and
b) a light chain variable region (V_{L}) comprising VLCDR1, VLCDR2 and VLCDR3 amino acid sequences; wherein the combination of VHCDR1, VHCDR2, VHCDR3, VLCDR1, VLCDR2 and VLCDR3 amino acid sequences are shown in any of the rows in Table 1.

2. The antigen-binding molecule of claim 1, wherein the antigen-binding molecule specifically binds to ALPPL2 and/or ALPP or a cell expressing ALPPL2 and/or ALPP with an affinity of between about 14 pm to about 10 nM.

3. The antigen-binding molecule of claim 1, wherein the antigen-binding molecule specifically binds to rhesus macaque ALPPL2/ALPP ortholog.

4. The antigen-binding molecule of claim 1, wherein the antigen-binding molecule comprises a) a heavy chain variable region (V_{H}) comprising SEQ ID NO: 115, SEQ ID NO: 116 and SEQ ID NO: 117; and b) a light chain variable region (V_{L}) comprising SEQ ID NO: 118, SEQ ID NO: 119 and SEQ ID NO: 120.

5. The antigen-binding molecule of claim 1, wherein the antigen-binding molecule comprises:
a) a V_{H} amino acid sequence having at least 90% (including at least 91% to 100% and all integer percentages therebetween) sequence identity to a V_{H} amino acid sequence as shown in any of the rows in Table 2 or Table 3, and
b) a V_{L} amino acid sequence having at least 90% sequence identity (including at least 91% to 100% and all integer percentages therebetween) to a V_{L} amino acid sequence as shown in the same row as the V_{H} amino acid sequence in Table 2 or Table 3.

6. The antigen-binding molecule of claim 1, wherein the antigen-binding molecule comprises:
a) a V_{H} amino acid sequence having at least 90% (including at least 91% to 100% and all integer percentages therebetween) sequence identity to SEQ ID NO: 291, and
b) a V_{L} amino acid sequence having at least 90% sequence identity (including at least 91% to 100% and all integer percentages therebetween) to SEQ ID NO: 292.

7. The antigen-binding molecule of claim 1, wherein the antigen-binding molecule does not bind to ALPP.

8. The antigen binding molecule of claim 1, wherein the antigen-binding molecule is an antibody or antigen-binding fragment thereof or a chimeric antigen receptor (CAR).

9. The antigen binding molecule of claim 8, wherein the antibody or antigen-binding fragment thereof is humanized or chimerized.

10. The antigen-binding molecule of claim 8, wherein the antibody or antigen-binding fragment thereof is a full-length antibody, a substantially intact antibody, a Fab fragment, scFab, Fab′, a single chain variable fragment (scFv) or a one-armed antibody.

11. The antigen-binding molecule of claim 8, wherein the antibody is a bispecific or trispecific antibody.

12. The antigen-binding molecule of claim 11, wherein the bispecific antibody comprises a first antigen-binding site that specifically binds ALPPL2 and a second antigen-binding site that specifically binds CD3.

13. A chimeric molecule comprising an antigen-binding molecule according to any one of claim 1 to 12 and a heterologous moiety.

14. The chimeric molecule of claim 13, wherein the heterologous moiety is a detectable moiety, a half-life extending moiety, or a therapeutic moiety.

15. The chimeric molecule of claim 14, wherein the therapeutic moiety is monomethyl auristatin F (MMAF) or monomethyl auristatin E (MMAE).

16. An isolated polynucleotide comprising a nucleic acid sequence encoding the antigen-binding molecule according to any one of claims 1 to 10, or the chimeric molecule of any one of claims 13 to 15.

17. A construct comprising a nucleic acid sequence encoding the antigen-binding molecule according to any one of claims 1 to 12, or the chimeric molecule of any one of claims 13 to 15 in operable connection with one or more control sequences.

18. A host cell that contains the construct of claim 17.

19. A pharmaceutical composition comprising the antigen-binding molecule according to any one of claims 1 to 12, or the chimeric molecule of any one of claims 13 to 15, and a pharmaceutically acceptable carrier.

20. A method for reducing the expression or activity of ALPPL2 in a cancer cell, the method comprising contacting the cancer cell with an antigen-binding molecule according to any one of claims 1 to 12 or a chimeric molecule according to any one of claims 13 to 15.

21. A method for reducing or inhibiting proliferation, survival and viability of a tumor in a subject, the method comprising administering an antigen-binding molecule according to any one of claims 1 to 12 or a chimeric molecule according to any one of claims 13 to 15 to the subject.

22. A method of treating cancer in a subject, wherein the method comprises administering an antigen-binding molecule according to any one of claims 1 to 12 or a chimeric molecule according to any one of claims 13 to 15 to the subject.

23. The method of claim 22, wherein the cancer is colorectal, endometrial, gastric, mesothelioma, ovarian, pancreatic or testicular cancer.

24. An antigen-binding molecule according to any one of claim 1 to 12 or a chimeric molecule according to any one of claims 13 to 15 for use in the treatment of cancer.

25. Use of an antigen-binding molecule according to any one of claims 1 to 12 or a chimeric molecule according to any one of claims 13 to 15 in the manufacture of a medicament for the treatment of cancer.

26. A method of treating a disease or condition associated with the undesired expression of ALPPL2 in a subject, wherein the method comprises administering an antigen-binding molecule according to any one of claims 1 to 12 or a chimeric molecule according to any one of claims 13 to 15 to the subject.

27. A kit for detecting cancer, the kit comprising an antigen-binding molecule according to any one of claims 1 to 12 or a chimeric molecule according to any one of claims 13 to 15.

28. A method of determining the likelihood of a cancer in a subject, wherein the method comprises detecting ALPPL2 in a sample obtained from the subject, wherein an increased level of ALPPL2 in the sample as compared to a reference indicates the likelihood of cancer in the subject.

29. The method of claim 28, wherein the method comprises detecting ALPPL2 with an antigen-binding molecule according to any one of claims 1 to 12 or a chimeric molecule according to any one of claims 13 to 15.

30. A method of treating a cancer in a subject, wherein the method comprises a) detecting ALPPL2 in a sample obtained from the subject, wherein an increased level of ALPPL2 in the sample as compared to a reference indicates an increased likelihood of cancer in the subject; and b) treating a subject found to have an increased likelihood of cancer.

31. The method of claim 30, wherein the method comprises detecting ALPPL2 with an antigen-binding molecule according to any one of claims 1 to 12 or a chimeric molecule according to any one of claims 13 to 15.

32. The method of claim 31, wherein the method comprises treating the subject with an antigen-binding molecule according to any one of claims 1 to 12 or a chimeric molecule according to any one of claims 13 to 15.

33. A method of identifying a subject who is likely to be responsive to treatment with an anti-ALPPL2 antibody, the method comprising detecting ALPPL2 in a sample obtained from the subject, wherein an increased level of ALPPL2 indicates that the subject is likely to be responsive to treatment with the ALPPL2 antibody.

34. The method of claim 33, wherein the method comprises detecting ALPPL2 with an antigen-binding molecule according to any one of claims 1 to 12 or a chimeric molecule according to any one of claims 13 to 15.

35. A method of identifying and treating a subject who is likely to be responsive to treatment with an anti-ALPPL2 antibody, the method comprising a) detecting ALPPL2 in a sample obtained from the subject, wherein an increased level of ALPPL2 indicates that the subject is likely to be responsive to treatment with the ALPPL2 antibody; and b) treating the subject found likely to be responsive to treatment with the ALPPL2 antibody.

36. A method for preparing an antigen-binding molecule that specifically binds ALPPL2 but not ALPL or ALPI, the method comprising:
a) immunizing an animal, preferentially a rabbit, with ALPPL2,
b) isolating from the animal a B-cell that binds specifically to ALPPL2 but not ALPL or ALPI, and
c) determining the amino acid sequence of the antibody that is expressed by the B-cell.
